(19) United States
(12) Patent Application Publication
(10) Pub. No.: US 2009/0192158 A1
(43) Pub. Date: Jul. 30, 2009

(54) Methods for Treating or Preventing Neoplasias

(76) Inventors: Stacia Kargman, Montreal (CA); Gary O'Neill, Dollard-des-Ormauex (CA); Daigen Xu, Pointe Claire (CA)

Correspondence Address: MERCK AND CO., INC, P O BOX 2000, RAHWAY, NJ, 07065-0907, US

(21) Appl. No.: 12/226,169

(22) PCT Filed: Apr. 30, 2007
(86) PCT No.: PCT/CA2007/000735

§ 371 Date: Oct. 8, 2008

Related U.S. Application Data
(60) Provisional application No. 60/796,905, filed on May 2, 2006.

(51) Int. Cl. A61K 31/5377; A61P 35/00; A61K 31/4184; A61K 31/4439; A61K 31/4245

(52) U.S. Cl. 514/235.5; 514/394; 514/338; 514/381; 514/364

(57) ABSTRACT

The present invention is directed to a method for treating or preventing a neoplasia in a human patient in need of such treatment comprising administering to the patient a compound that inhibits microsomal prostaglandin E synthase-1 in an amount that is effective for treating or preventing the neoplasia.

### BACKGROUND OF THE INVENTION

Modulation of prostaglandin metabolism is at the center of current anti-inflammatory therapies. NSAIDs and COX-2 inhibitors block the activity of cyclooxygenases and their ability to convert arachidonic acid (AA) into prostaglandin (PG) H2. PGH2 can be subsequently metabolized by terminal prostaglandin synthases to the corresponding biologically active PGs, namely, PGI2, thromboxane (Tx) A2, PGD2, PGF2α, and PGE2. A combination of pharmacological, genetic, and neutralizing antibody approaches demonstrates the importance of PGE2 in inflammation. In many respects, disruption of PGE2-dependent signalling in animal models of inflammation can be as effective as treatment with NSAIDs or COX-2 inhibitors. The conversion of PGH2 to PGE2 by prostaglandin E synthases (PGES) may therefore represent a pivotal step in the propagation of inflammatory stimuli.

Microsomal prostaglandin E synthase-1 (mPGES-1) is an inducible PGES after exposure to pro-inflammatory stimuli. mPGES-1 is induced in the periphery and in the CNS by inflammation and represents therefore a novel target for acute and chronic inflammatory disorders. The rationale for the development of specific mPGES-1 inhibitors revolves around the hypothesis that the therapeutic utility of NSAIDs and Cox-2 inhibitors would be largely due to inhibition of pro-inflammatory PGE2 while the side effect profile would be largely due to inhibition of other prostaglandins.

In addition to their anti-inflammatory effects, NSAIDs and COX-2 inhibitors are also effective in preventing or treating benign or malignant neoplasia in animal models and humans. It is believed that COX-2 promotes the formation, growth and/or metastasis of neoplasia by producing PGE2. mPGES-1 is often co-expressed with COX-2 in benign and cancerous neoplastic tissues of various origins, suggesting that mPGES-1 act as the PGE2-producing synthase downstream of COX-2 in neoplasia. In support of this view, treatment of a human colon tumor cell line with an mPGES-1 anti-sense oligonucleotide or a prototypic Merck mPGES-1 inhibitor MK-886 inhibits PGE2 formation and cell proliferation (data published by outside scientists). Thus like COX-2, mPGES-1 represents a useful target for both benign and malignant neoplasia.

In addition to their anti-inflammatory effects, NSAIDs and COX-2 inhibitors are also effective in preventing or treating benign or malignant neoplasia in animal models and humans. It is believed that COX-2 promotes the formation, growth and/or metastasis of neoplasia by producing PGE2. mPGES-1 is often co-expressed with COX-2 in benign and cancerous neoplastic tissues of various origins, suggesting that mPGES-1 act as the PGE2-producing synthase downstream of COX-2 in neoplasia. In support of this view, treatment of a human colon tumor cell line with an mPGES-1 anti-sense oligonucleotide or a prototypic Merck mPGES-1 inhibitor MK-886 inhibits PGE2 formation and cell proliferation. See Kamei et al., The Journal of Biological Chemistry, vol. 278, no. 21, pp. 19396-19405, 2003. Thus like COX-2, mPGES-1 represents a useful target for both benign and malignant neoplasia.

The present invention is directed to methods for treating or preventing a neoplasia by administering a compound that inhibits microsomal prostaglandin E synthase-1 in an amount that is effective to treat or prevent a neoplasia.

### SUMMARY OF THE INVENTION

The present invention is directed to a method for treating or preventing a neoplasia in a human patient in need of such treatment comprising administering to the patient a compound that inhibits microsomal prostaglandin E synthase-1 in an amount that is effective for treating or preventing the neoplasia.

### BRIEF DESCRIPTION OF THE DRAWINGS

FIG. 1—This figure demonstrates endogenous expression of mPGES-1 in the microsomal/membrane fraction of human lung adenocarcinoma cells (A549). The expression of mPGES-1 is induced by the cytokine IL-1β. Example 81, a selective mPGES-1 inhibitor, inhibits IL-1β induced mPGES-1 induced PGE2 synthesis with an IC₅₀ of 3.29 nM.

### DETAILED DESCRIPTION OF THE INVENTION

The invention is directed to a method for treating or preventing a neoplasia in a human patient in need of such treatment comprising administering to the patient a compound that inhibits microsomal prostaglandin E synthase-1 in an amount that is effective for treating or preventing the neoplasia.

In an embodiment of the invention, the neoplasia is a benign tumor, growth or polyp. Within this embodiment, the neoplasia is selected from the group consisting of: squamous cell papilloma, basal cell tumor, transitional cell papilloma, adenoma, gastrinoma, cholangiocellular adenoma, hepatocellular adenoma, renal tubular adenoma, oncocytoma, glomus tumor, melanocytic nevus, fibroma, myxoma, lipoma, leiomyoma, rhabdomyoma, benign teratoma, hemangioma, osteoma, chondroma and meningioma.

In another embodiment of the invention, the neoplasia is a cancerous tumor, growth or polyp. With the embodiment, the neoplasia is selected from the group consisting of: squamous cell carcinoma, basal cell carcinoma, transitional cell carcinoma, adenocarcinoma, malignant gastrinoma, cholangiocelleular carcinoma, hepatocellular carcinoma, renal cell carcinoma, malignant melanoma, fibrosarcoma, myxosarcoma, liposarcoma, leimyosarcoma, rhabdomyosarcoma, malignant teratoma, hemangiosarcoma, Kaposi sarcoma, lymphangiosarcoma, osteosarcoma, chondrosarcoma, malignant meningioma, non-Hodgkin lymphoma, Hodgkin lymphoma and leukemia.

In another embodiment, the neoplasia is cancer selected from the group consisting of: brain cancer, bone cancer, basal cell carcinoma, adenocarcinoma, lip cancer, mouth cancer, esophogeal cancer, small bowel cancer, stomach cancer, colon cancer, rectal cancer, liver cancer, bladder cancer, pancreas cancer, ovary cancer, cervical cancer, lung cancer, breast cancer, head and neck cancer, skin cancer, prostate cancer, gall bladder cancer, thyroid cancer and renal cell carcinoma. Within this embodiment, the cancer is selected from the group consisting of: colon cancer, esophageal cancer, stomach cancer, breast cancer, head and neck cancer, skin cancer, lung cancer, liver cancer, gall bladder, pancreas cancer, bladder cancer, cervical cancer, prostate cancer, thyroid cancer and brain cancer.

In another embodiment, the invention encompasses the above methods wherein the compound that inhibits microsomal prostaglandin E synthase-1 is a genus represented by Formula I

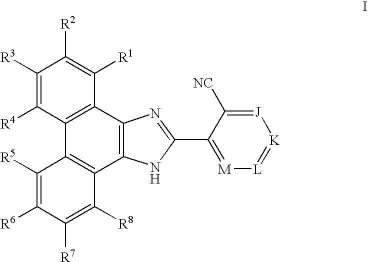

or a prodrug thereof, or a pharmaceutically acceptable salt of said compound or prodrug, wherein:
J is selected from the group consisting of —C(X²)— and —N—,
K is selected from the group consisting of —C(X³)— and —N—,
L is selected from the group consisting of —C(X⁴)— and —N—, and
M is selected from the group consisting of —C(X⁵)— and —N—, with the proviso that at least one of J, K, L or M is other than —N—;
X², X³, X⁴ and X⁵ are independently selected from the group consisting of: (1) H; (2) —CN; (3) F;
(4) Cl; (5) Br; (6) I; (7) —OH; (8) —N₃; (9) C₁₋₆alkyl, C₂₋₆alkenyl or C₂₋₆alkynyl, wherein one or more of the hydrogen atoms attached to said C₁₋₆alkyl, C₂₋₆alkenyl or C₂₋₆alkynyl may be replaced with a fluoro atom, and said C₁₋₆alkyl, C₂₋₆alkenyl or C₂₋₆alkynyl may be optionally substituted with a hydroxy group; (10) C₁₋₄alkoxy; (11) NR⁹R¹⁰—C(O)—C₁₋₄alkyl-O—; (12) C₁₋₄alkyl-S(O)ₖ—; (13) —NO₂; (14) C₃₋₆cycloalkyl, (15) C₃₋₆cycloalkoxy; (16) phenyl, (17) carboxy; and (18) C₁₋₄alkyl-O—C(O)—;
R¹, R², R³, R⁴, R⁵, R⁶, R⁷ and R⁸ are independently selected from the group consisting of: (1) H; (2) F; (3) Cl; (4) Br; (5) I; (6) —CN; (7) C₁₋₁₀alkyl or C₂₋₁₀alkenyl, wherein one or more of the hydrogen atoms attached to said C₁₋₁₀alkyl or C₂₋₁₀alkenyl may be replaced with a fluoro atom, or two hydrogen on adjacent carbon atoms may be joined together and replaced with —CH₂— to form a cyclopropyl group, or two hydrogen atoms on the same carbon atom may be replaced and joined together to form a spiro C₃₋₆cycloalkyl group, and wherein said C₁₋₁₀alkyl or C₂₋₁₀alkenyl may be optionally substituted with one to three substituents independently selected from the group consisting of: —OH, acetyl, methoxy, ethenyl, R¹¹—O—C(O)—, R³⁵—N(R³⁶)—, R³⁷—N(R³⁸)—C(O)—, cyclopropyl, pyrrolyl, imidiazolyl, pyridyl and phenyl, said pyrrolyl, imidiazolyl, pyridyl and phenyl optionally substituted with C₁₋₄alkyl or mono-hydroxy substituted C₁₋₄alkyl; (8) C₃₋₆cycloalkyl; (9) R¹²—O—; (10) R¹³—S(O)ₖ—, (11) R¹⁴—S(O)ₖ—N(R¹⁵)—; (12) R¹⁶—C(O)—; (13) R¹⁷—N(R¹⁸)—; (14) R¹⁹—N(R²⁰)—C(O)—; (15) R²¹—N(R²²)—S(O)ₖ—; (16) R²³—C(O)—N(R²⁴)—; (17) Z-C≡C; (18) —CH₃)C═N—OH or —(CH₃)C═N—OCH₃; (19) R³⁴—O—C(O)—; (20) R³⁹—C(O)—O—; and (21) phenyl, naphthyl, pyridyl, pyradazinyl, pyrimidinyl, pyrazinyl, pyrrolyl, pyrazolyl, imidazolyl, triazolyl, tetrazolyl, oxazolyl, isoxazolyl, oxadiazolyl, thienyl or furyl, each optionally substituted with a substituent independently selected from the group consisting of: F, Cl, Br, I, C₁₋₄alkyl, phenyl, methylsulfonyl, methylsulfonylamino, R²⁵—O—C(O)— and R²⁶—N(R²⁷)—, said C₁₋₄alkyl optionally substituted with 1 to 3 groups independently selected from halo and hydroxy;
each Z is independently selected from the group consisting of: (1) H; (2) C₁₋₆alkyl, wherein one or more of the hydrogen atoms attached to said C₁₋₆alkyl may be replaced with a fluoro atom, and wherein
C₁₋₆alkyl is optionally substituted with one to three substituents independently selected from: hydroxy, methoxy, cyclopropyl, phenyl, pyridyl, pyrrolyl, R²⁸—N(R²⁹)— and R³⁰—O—C(O)—; (3) —(CH₃)C═N—OH or —CH₃)C—N—OCH₃; (4) R³¹—C(O)—; (5) phenyl; (6) pyridyl or the N-oxide thereof; (7) C₃₋₆cycloalkyl, optionally substituted with hydroxy; (8) tetrahydropyranyl, optionally substituted with hydroxy; and (9) a five-membered aromatic heterocycle containing 1 to 3 atoms independently selected from O, N or S and optionally substituted with methyl;
each R⁹, R¹⁰, R¹⁵, R²⁴ and R³² is independently selected from the group consisting of: (1) H; and
(2) C₁₋₄alkyl;
each R¹¹, R¹², R¹³, R¹⁴, R¹⁶, R²³, R²⁵, R³⁰, R³¹, R³⁴ and R³⁹ is independently selected from the group consisting of: (1) H; (2) C₁₋₄alkyl, (3) C₃₋₆cycloalkyl; (4) C₃₋₆cycloalkyl-C₁₋₄alkyl- (5) phenyl, (6) benzyl; and (7) pyridyl; said C₁₋₄alkyl, C₃₋₆cycloalkyl, C₃₋₆cycloalkyl-C₁₋₄alkyl-, phenyl, benzyl and pyridyl may each be optionally substituted with 1 to 3 substituents independently selected from the group consisting of: OH, F, Cl, Br and I, and wherein said C₁₋₄alkyl may be further substituted with oxo or methoxy or both;
each R¹⁷, R¹⁸, R¹⁹, R²⁰, R²¹, R²², R²⁶, R²⁷, R²⁸, R²⁹, R³⁵, R³⁶, R³⁷ and R³⁸ is independently selected from the group consisting of: (1) H; (2) C₁₋₆alkyl; (3) C₁₋₆alkoxy; (4) OH and (5) benzyl or 1-phenylethyl; and R¹⁷ and R¹⁸, R¹⁹ and R²⁰, R²¹ and R²², R²⁶ and R²⁷, and R²⁸ and R²⁹, R³⁵ and R³⁶, and R³⁷ and R³⁸ may be joined together with the nitrogen atom to which they are attached to form a monocyclic ring of 5 or 6 carbon atoms, optionally containing one or two atoms independently selected from —O—, —S(O)ₖ— and —N(R³²)—; and
each k is independently 0, 1 or 2.

In another embodiment, the invention encompasses the above methods wherein the compound that inhibits microsomal prostaglandin E synthase-1 is a first sub-genus of compounds within the genus represented by Formula I

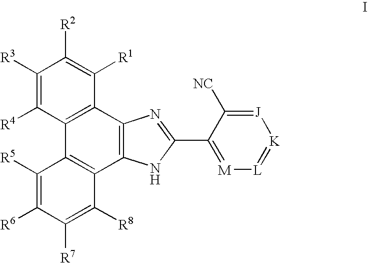

or a prodrug thereof, or a pharmaceutically acceptable salt of said compound or prodrug, wherein:
J is selected from the group consisting of <(X²)— and —N—,
K is selected from the group consisting of —C(X³)— and —N—,
L is selected from the group consisting of —C(X⁴)— and —N—, and
M is selected from the group consisting of —C(X⁵)— and —N—,
with the proviso that at least one of J, K, L or M is other than —N—;
X², X³, X⁴ and X⁵ are independently selected from the group consisting of: (1) H; (2) —CN; (3) F;
(4) Cl; (5) Br; (6) I; (7) —OH; (8) —N₃; (9) C₁₋₆alkyl, C₂₋₆alkenyl Or C₂₋₆alkynyl, wherein one or more of the hydrogen atoms attached to said C₁₋₆alkyl, C₂₋₆alkenyl or C₂₋₆alkynyl may be replaced with a fluoro atom, and said C₁₋₆alkyl, C₂₋₆alkenyl or C₂₋₆alkynyl may be optionally substituted with a hydroxy group; (10) C₁₋₄alkoxy; (11) NR⁹R¹⁰—C(O)—C₁₋₄alkyl-O—; (12) C₁₋₄alkyl-S(O)ₖ—; (13) —NO₂; (14) C₃₋₆cycloalkyl, (15) C₃₋₆cycloalkoxy; (16) phenyl, (17) carboxy; and (18) C₁₋₄alkyl-O—C(O)—;
R¹, R², R³, R⁴, R⁵, R⁶, R⁷ and R⁸ are independently selected from the group consisting of: (1) H; (2) F; (3) Cl; (4) Br; (5) I; (6) —CN; (7) C₁₋₆alkyl or C₂₋₆alkenyl, wherein one or more of the hydrogen atoms attached to said C₁₋₆alkyl or C₂₋₆alkenyl may be replaced with a fluoro atom, and wherein said C₁₋₆alkyl or C₂₋₆alkenyl may be optionally substituted with one to three substituents independently selected from the group consisting of: —OH, methoxy, R¹¹—O—C(O)—, cyclopropyl, pyridyl and phenyl; (8) C₃₋₆cycloalkyl; (9) R¹²—O—; (10) R¹³—S(O)ₖ—, (11) R¹⁴—S(O)ₖ—N(R¹⁵)—; (12) R¹⁶—C(O)—; (13) R¹⁷—N(R¹⁸)—; (14) R¹⁹—N(R²⁰)—C(O)—; (15) R²¹—N(R²²)—S(O)ₖ—; (16) R²³—C(O)—N(R²⁴)—; (17) Z-C≡C;
(18) —CH₃)C═N—OH or —(CH₃)C═N—OCH₃; and (19) phenyl, naphthyl, pyridyl, pyradazinyl, pyrimidinyl, pyrazinyl, pyrrolyl, pyrazolyl, imidazolyl, triazolyl, tetrazolyl, oxazolyl, isoxazolyl, oxadiazolyl, thienyl or furyl, each optionally substituted with a substituent independently selected from the group consisting of: F, Cl, Br, I, C₁₋₄alkyl, phenyl, methylsulfonyl, methylsulfonylamino, R²⁵—O—C(O)— and R²⁶—N(R²⁷)—, said C₁₋₄alkyl optionally substituted with 1 to 3 groups independently selected from halo and hydroxy;
each Z is independently selected from the group consisting of: (1) H; (2) C₁₋₆alkyl, wherein one or more of the hydrogen atoms attached to said C₁₋₆alkyl may be replaced with a fluoro atom, and wherein
C₁₋₆alkyl is optionally substituted with one to three substituents independently selected from: hydroxy, methoxy, cyclopropyl, phenyl, pyridyl, pyrrolyl, R²⁸—N(R²⁹)— and R³⁰—O—C(O)—; (3) —(CH₃)C═N—OH or —(CH₃)C═N—OCH₃; (4) R³¹—C(O)—; (5) phenyl; (6) pyridyl or the N-oxide thereof; (7) C₃₋₆cycloalkyl, optionally substituted with hydroxy; (8) tetrahydropyranyl, optionally substituted with hydroxy; and (9) a five-membered aromatic heterocycle containing 1 to 3 atoms independently selected from O, N or S and optionally substituted with methyl;
each R⁹, R¹⁰, R¹⁵, R²⁴ and R³² is independently selected from the group consisting of: (1) H; and
(2) C₁₋₄alkyl;
each R¹¹, R¹², R¹³, R¹⁴, R¹⁶, R²³, R²⁵, R³⁰ and R³¹ is independently selected from the group consisting of: (1) H; (2) C₁₋₄alkyl, (3) C₃₋₆cycloalkyl; (4) phenyl, (5) benzyl; and (6) pyridyl; said C₁₋₄alkyl, C₃₋₆cycloalkyl, phenyl, benzyl and pyridyl may each be optionally substituted with 1 to 3 substituents independently selected from the group consisting of: OH, F, Cl, Br and I;
each R¹⁷, R¹⁸, R¹⁹, R²⁰, R²¹, R²², R²⁶, R²⁷, R²⁸ and R²⁹ is independently selected from the group consisting of: (1) H; (2) C₁₋₆alkyl; (3) C₁₋₆alkoxy; (4) OH and (5) benzyl or 1-phenylethyl; and R¹⁷ and R¹⁸, R¹⁹ and R²⁰, R²¹ and R²², R²⁶ and R²⁷, and R²⁸ and R²⁹ may be joined together with the nitrogen atom to which they are attached to form a monocyclic ring of 5 or 6 carbon atoms, optionally containing one or two atoms independently selected from —O—, —S(O)ₖ— and —N(R³²)—; and
each k is independently 0, 1 or 2.

In another embodiment, the invention encompasses the above methods wherein the compound that inhibits microsomal prostaglandin E synthase-1 is a first class of compounds within the first sub-genus represented by Formula A

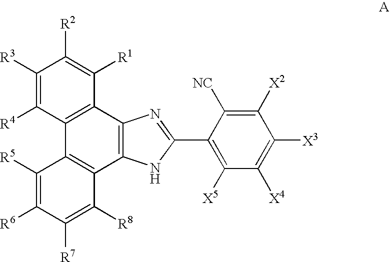

or a prodrug thereof, or a pharmaceutically acceptable salt of said compound or prodrug.

In another embodiment, the invention encompasses the above methods the compound that inhibits microsomal prostaglandin E synthase-1 is a first sub-class of compounds within the first class represented by Formula A wherein:

X², X³, X⁴ and X⁵ are independently selected from the group consisting of: (1) H; (2) —CN; (3) F;

### (4) Cl; (5) Br; and (6) 1.

In another embodiment, the invention encompasses the above methods wherein the compound that inhibits microsomal prostaglandin E synthase-1 is a second sub-class of compounds within the first class represented by Formula A wherein X², X³ and X⁴ are H, and X⁵ is other than H.

In another embodiment, the invention encompasses the above methods the compound that inhibits microsomal prostaglandin E synthase-1 is within the second sub-class represented by Formula A wherein X⁵ is —CN.

In another embodiment, the invention encompasses the above methods wherein the compound that inhibits microsomal prostaglandin E synthase-1 is a second class of compounds within the first sub-genus represented by Formula A wherein at least one of R¹ or R⁸ is other than H.

In another embodiment, the invention encompasses the above methods wherein the compound that inhibits microsomal prostaglandin E synthase-1 is a third class of compounds within the first sub-genus represented by Formula A wherein at least one of R² or R⁷ is other than H.

In another embodiment, the invention encompasses the above methods wherein the compound that inhibits microsomal prostaglandin E synthase-1 is a fourth class of compounds within the first sub-genus represented by Formula A wherein at least one of R⁴ or R⁵ is other than H.

In another embodiment, the invention encompasses the above methods wherein the compound that inhibits microsomal prostaglandin E synthase-1 is a fifth class of compounds within the first sub-genus represented by Formula A wherein:

at least one of R³ or R⁶ is other than H; and

### R¹, R², R⁴, R⁵, R⁷ and R⁸ are H.

In another embodiment, the invention encompasses the above methods wherein the compound that inhibits microsomal prostaglandin E synthase-1 is a first sub-class of compounds within the fifth class represented by Formula A wherein R³ and R⁶ are both other than H.

In another embodiment, the invention encompasses the above methods wherein the compound that inhibits microsomal prostaglandin E synthase-1 is within this first sub-class represented by Formula A wherein:

one of R³ or R⁶ is independently selected from the group consisting of: F, Cl, Br, and I; and
the other of R³ or R⁶ is Z-C≡C.

In another embodiment, the invention encompasses the above methods wherein the compound that inhibits microsomal prostaglandin E synthase-1 is a second sub-class of compounds within the fifth class represented by Formula A wherein: R³ and R⁶ are independently selected from the group consisting of: hydrogen, fluoro, chloro, bromo, iodo, cyano, methyl, ethyl, vinyl, cyclopropyl, —CO₂i-Pr, —CO₂CH₃, —SO₂CF₃, 3-pyridyl, acetyl,

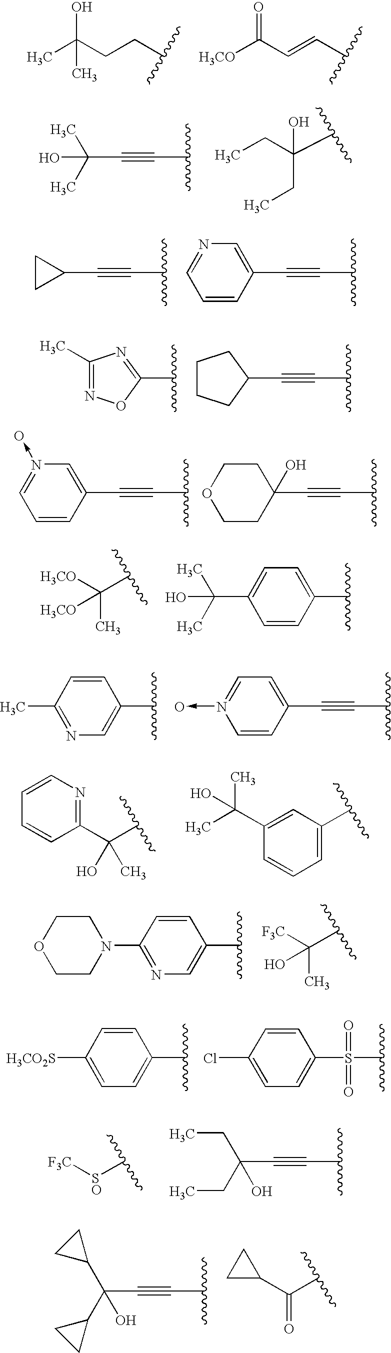

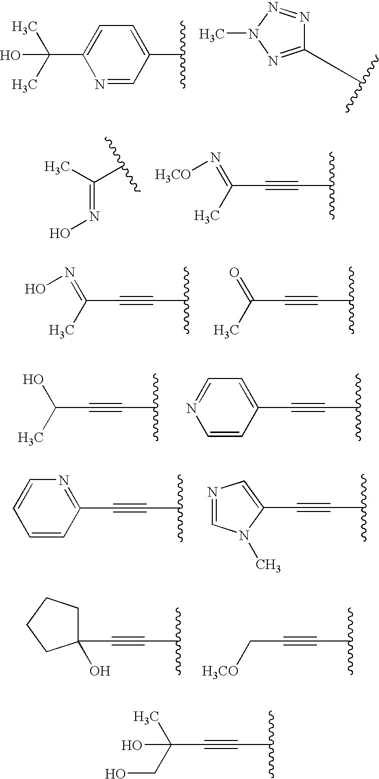

with the proviso that at least one of R³ or R⁶ is other than H.

In another embodiment, the invention encompasses the above methods the compound that inhibits microsomal prostaglandin E synthase-1 is a sixth class within the first sub-genus represented by Formula B:

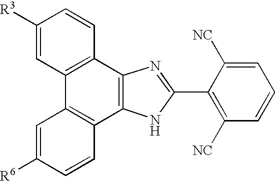

or a prodrug thereof, or a pharmaceutically acceptable salt of said compound or prodrug.

In another embodiment, the invention encompasses the above methods wherein the compound that inhibits microsomal prostaglandin E synthase-1 is a first sub-class within the sixth class represented by Formula B wherein:

one of R³ or R⁶ is independently selected from the group consisting of: F, Cl, Br, and I; and
the other of R³ or R⁶ is Z-C≡C.

In another embodiment, the invention encompasses the above methods wherein the compound that inhibits microsomal prostaglandin E synthase-1 is a second sub-genus which is a prodrug represented by Formula C

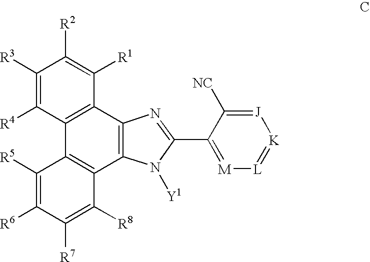

or a pharmaceutically acceptable salt thereof, wherein:
Y¹ is selected from the group consisting of: (1) C₁₋₆alkyl; (2) PO₄—C₁₋₄alkyl-; (3) C₁₋₄alkyl-C(O)—O—CH₂—, wherein the C₁₋₁₄alkyl portion is optionally substituted with R³³—O—C(O)—; and (4) C₁₋₁₄alkyl-O—C(O)—; and
R³³ is selected from the group consisting of: (1) H; (2) C₁₋₄alkyl, (3) C₃₋₆cycloalkyl; (4) phenyl; (5) benzyl; and (6) pyridyl; said C₁₋₄alkyl, C₃₋₆cycloalkyl, phenyl, benzyl and pyridyl may each be optionally substituted with 1 to 3 substituents independently selected from the group consisting of: OH, F, Cl, Br and I.

In another embodiment, the invention encompasses the above methods wherein the compound that inhibits microsomal prostaglandin E synthase-1 is selected from one of the following tables:

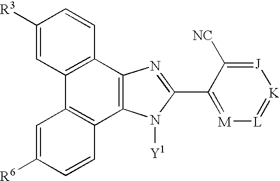

| Ex | R³/R⁶ | R⁶/R³ | J | K | L | M | Y¹ |
|---|---|---|---|---|---|---|---|
| 1 | Cl | Br | CH | CH | CH | CF | H |
| 2 | H | H | CH | CH | CH | CH | H |
| 3 | CN | 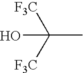 | CH | CH | CH | CF | H |
| 4 | Cl | 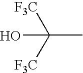 | CH | CH | CH | CF | H |
| 5 | Cl | H | CH | CH | CH | CF | H |
| 6 | CN | H | CH | CH | CH | CF | H |
| 7 | CN | 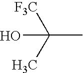 | CH | CH | CH | CF | H |
| 8 | Cl | 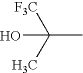 | CH | CH | CH | CF | H |
| 9 | Br | Br | CH | CH | CH | CF | H |
| 10 | H | H | CH | CH | CH | CCl | H |
| 11 | H | H | CH | CH | CH | CCN | H |
| 12 | 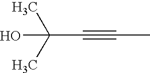 | Br | CH | CH | CH | CF | H |
| 13 | 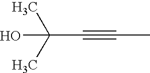 | 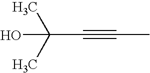 | CH | CH | CH | CF | H |
| 14 | 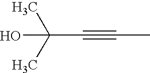 | Cl | CH | CH | CH | CF | H |
| 15 | 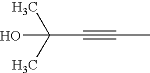 | I | CH | CH | CH | CF | H |
| 16 | H | H | CH | CH | CH | CBr | H |
| 17 | H | H | CH | CH | CH | CF | H |
| 18 | H | H | CH | N | CH | CCl | H |
| 19 | 3-pyridyl | 3-pyridyl | CH | CH | CH | CF | H |
| 20 | Cl | 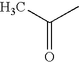 | CH | CH | CH | CF | H |
| 21 | Cl | 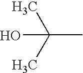 | CH | CH | CH | CF | H |
| 22 | 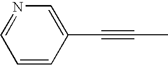 | Br | CH | CH | CH | CF | H |
| 23 | Cl | H | CH | N | CH | CCN | H |
| 24 | H | H | CH | N | CH | CCN | H |
| 25 | Cl | H | CH | CH | CH | CCN | H |
| 26 | H | H | CH | N | CH | CH | H |
| 27 | 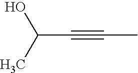 | Br | CH | CH | CH | CF | H |
| 28 | 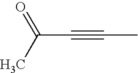 | Br | CH | CH | CH | CF | H |
| 29 | 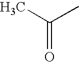 | 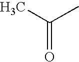 | CH | CH | CH | CF | H |
| 30 | 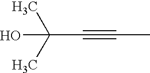 | 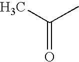 | CH | CH | CH | CF | H |
| 31 | H | H | N | CH | CH | N | H |
| 32 | H | H | N | CH | CH | CH | H |
| 33 | Br | 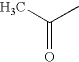 | CH | CH | CH | CF | H |
| 34 | I | I | CH | CH | CH | CF | H |
| 35 | Br | 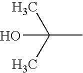 | CH | CH | CH | CF | H |
| 36 | Br | Cl | CH | CH | CH | CCN | H |
| 37 | Cl | 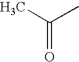 | CH | CH | CH | CBr | H |
| 38 | Cl | 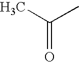 | CH | CH | CH | CCN | H |
| 39 | I | I | CH | CH | CH | CCN | H |
| 40 | 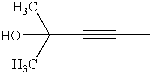 | Cl | CH | CH | CH | CCN | H |
| 41 | Cl | 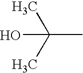 | CH | CH | CH | CCN | H |
| 42 | 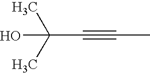 | I | CH | CH | CH | CCN | H |
| 43 | 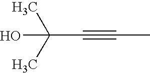 | 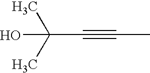 | CH | CH | CH | CCN | H |
| 44 | H | H | CH | CH | CH | CCN | CO₂Et |
| 45 | H | H | CH | CH | CH | CCN | 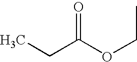 |
| 46 | 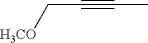 | Cl | CH | CH | CH | CCN | H |
| 47 | 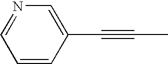 | Cl | CH | CH | CH | CCN | H |
| 48 | 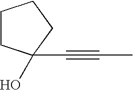 | Cl | CH | CH | CH | CCN | H |
| 49 | 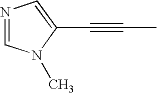 | Cl | CH | CH | CH | CCN | H |
| 50 | 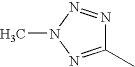 | Cl | CH | CH | CH | CCN | H |
| 51 | Cl | 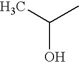 | CH | CH | CH | CCN | H |
| 52 | 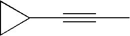 | Cl | CH | CH | CH | CCN | H |
| 53 | 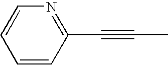 | Cl | CH | CH | CH | CCN | H |
| 54 | 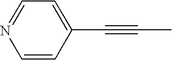 | Cl | CH | CH | CH | CCN | H |
| 55 | 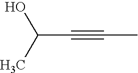 | Cl | CH | CH | CH | CCN | H |
| 56 | 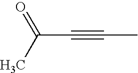 | Cl | CH | CH | CH | CCN | H |
| 57 | 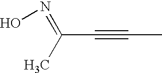 | Cl | CH | CH | CH | CCN | H |
| 58 | 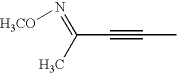 | Cl | CH | CH | CH | CCN | H |
| 59 | H | H | CH | CH | CH | CCN | 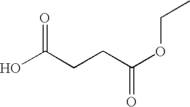 |
| 60 | H | H | CH | CH | CH | CCN | H₂PO₄CH₂ |
| 61 | 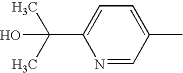 | Cl | CH | CH | CH | CCN | H |
| 62 | Cl | SO₂CH₃ | CH | CH | CH | CCN | H |
| 63 | Cl | 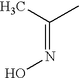 | CH | CH | CH | CCN | H |
| 64 | Br | H | CH | CH | CH | CCN | H |
| 65 | Cl | 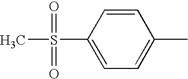 | CH | CH | CH | CCN | H |
| 66 | I | H | CH | CH | CH | CCN | H |
| 67 | CN | H | CH | CH | CH | CCN | H |
| 68 | cyclopropyl | Cl | CH | CH | CH | CCN | H |
| 69 | 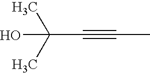 | 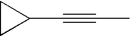 | CH | CH | CH | CCN | H |
| 70 | Cl | F | CH | CH | CH | CCN | H |
| 71 | Cl | 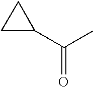 | CH | CH | CH | CCN | H |
| 72 | Cl | 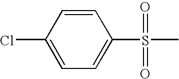 | CH | CH | CH | CCN | H |
| 73 | vinyl | H | CH | CH | CH | CCN | H |
| 74 | ethyl | H | CH | CH | CH | CCN | H |
| 75 | cyclopropyl | H | CH | CH | CH | CCN | H |
| 76 | Cl | 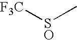 | CH | CH | CH | CBr | H |
| 77 | Cl | 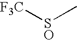 | CH | CH | CH | CCN | H |
| 78 | Cl | SO₂CF₃ | CH | CH | CH | CCN | H |
| 79 | 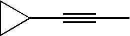 | H | CH | CH | CH | CCN | H |
| 80 | Cl | 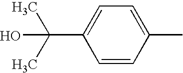 | CH | CH | CH | CCN | H |
| 81 | 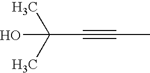 | Br | CH | CH | CH | CCN | H |
| 82 | Cl | 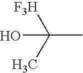 | CH | CH | CH | CCN | H |
| 83 | 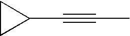 | 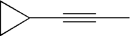 | CH | CH | CH | CCN | H |
| 84 | 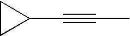 | 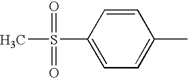 | CH | CH | CH | CCN | H |
| 85 | 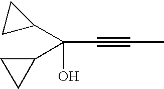 | Cl | CH | CH | CH | CCN | H |
| 86 | 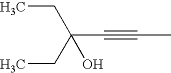 | Cl | CH | CH | CH | CCN | H |
| 87 | Br | 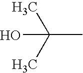 | CH | CH | CH | CCN | H |
| 88 | 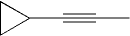 | 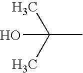 | CH | CH | CH | CCN | H |
| 89 | 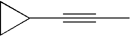 | CN | CH | CH | CH | CCN | H |
| 90 | 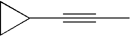 | CO₂CH₃ | CH | CH | CH | CCN | H |
| 91 | 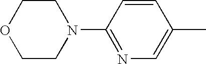 | Cl | CH | CH | CH | CCN | H |
| 92 | Cl | CN | CH | CH | CH | CCN | H |
| 93 | Cl | 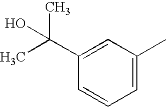 | CH | CH | CH | CCN | H |
| 94 | Br | 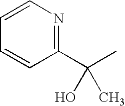 | CH | CH | CH | CCN | H |
| 95 | 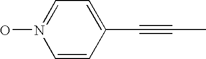 | Cl | CH | CH | CH | CCN | H |
| 96 | 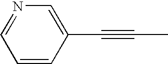 | 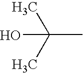 | CH | CH | CH | CCN | H |
| 97 | 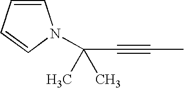 | Cl | CH | CH | CH | CCN | H |
| 98 | 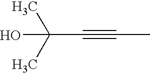 | Br | CH | CH | CH | CCl | H |
| 99 | 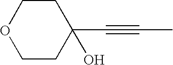 | Br | CH | CH | CH | CCl | H |
| 100 | Cl | CO₂i-Pr | CH | CH | CH | CCN | H |
| 101 | Cl | 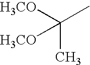 | CH | CH | CH | CF | H |
| 102 | 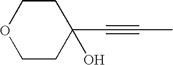 | Br | CH | CH | CH | CCN | H |
| 103 | 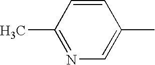 | Cl | CH | CH | CH | CCN | H |
| 104 | Br | 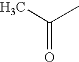 | CH | CH | CH | CCN | H |
| 105 | 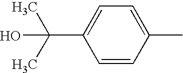 | Cl | CH | CH | CH | CCl | H |
| 106 | Br | 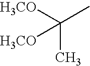 | CH | CH | CH | CCN | H |
| 107 | 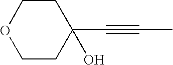 | Cl | CH | CH | CH | CCl | H |
| 108 | 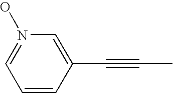 | Cl | CH | CH | CH | CCN | H |
| 109 | 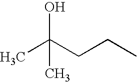 | Br | CH | CH | CH | CCN | H |
| 110 | 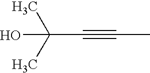 | Cl | CH | CH | CH | CCl | H |
| 111 | 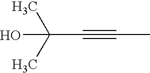 | 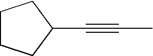 | CH | CH | CH | CCN | H |
| 112 | 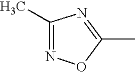 | Br | CH | CH | CH | CCN | H |
| 113 | 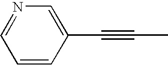 | 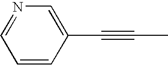 | CH | CH | CH | CCN | H |
| 114 | Et | 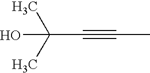 | CH | CH | CH | CCN | H |
| 115 | 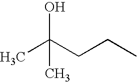 | 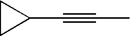 | CH | CH | CH | CCN | H |
| 116 | Br | 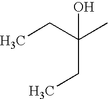 | CH | CH | CH | CCN | H |
| 117 | 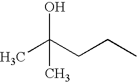 | Cl | CH | CH | CH | CCN | H |
| 118 | Br | CH₃ | CH | CH | CH | CCN | H |
| 119 | 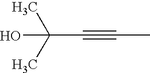 | CH₃ | CH | CH | CH | CCN | H |
| 120 | 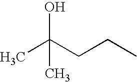 | CH₃ | CH | CH | CH | CCN | H |
| 121 | 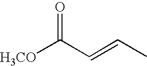 | Cl | CH | CH | CH | CCN | H |
| 122 | 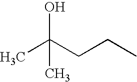 | H | CH | CH | CH | CCN | H |
| 123 | 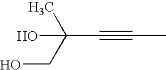 | Cl | CH | CH | CH | CCN | H |
| 124 | 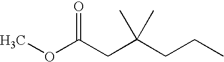 | Cl | CH | CH | CH | CCN | H |
| 125 | 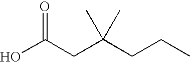 | Cl | CH | CH | CH | CCN | H |
| 126 | 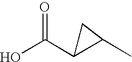 | Cl | CH | CH | CH | CCN | H |
| 127 | 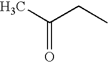 | Cl | CH | CH | CH | CCN | H |
| 128 | 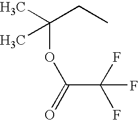 | Cl | CH | CH | CH | CCN | H |
| 129 | 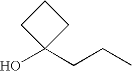 | Cl | CH | CH | CH | CCN | H |
| 130 | 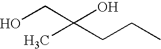 | Cl | CH | CH | CH | CCN | H |
| 131 | 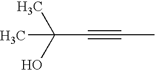 | Cl | CH | F | CH | CCN | H |
| 132 | 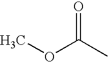 | 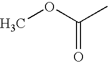 | CH | CH | CH | CCN | H |
| 133 | 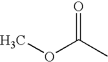 | 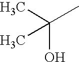 | CH | CH | CH | CCN | H |
| 134 | 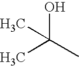 | 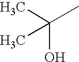 | CH | CH | CH | CCN | H |
| 135 | 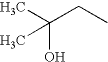 | Cl | CH | CH | CH | CCN | H |
| 136 | Br | Cl | CH | OH | CH | CCN | H |
| 137 | 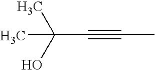 | Cl | CH | OH | CH | CCN | H |
| 138 | 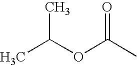 | 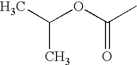 | CH | CH | CH | CCN | H |
| 139 | 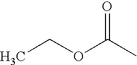 | 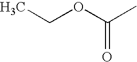 | CH | CH | CH | CCN | H |
| 140 | 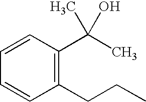 | 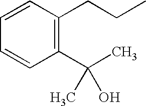 | CH | CH | CH | CCN | H |
| 141 | 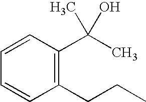 | Br | CH | CH | CH | CCN | H |
| 142 | 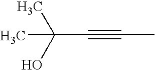 | Cl | CH | Cl | CH | CCN | H |
| 143 | 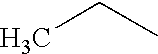 | 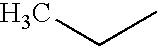 | CH | CH | CH | CCN | H |
| 144 | 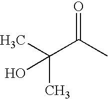 | Cl | CH | CH | CH | CCN | H |
| 145 | Br | 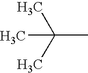 | CH | CH | CH | CCN | H |
| 146 | 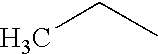 | 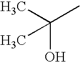 | CH | CH | CH | CCN | H |
| 147 | 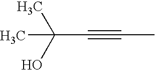 | 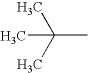 | CH | CH | CH | CCN | H |
| 148 | 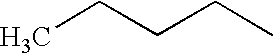 | 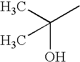 | CH | CH | CH | CCN | H |
| 149 | 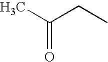 | 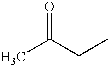 | CH | CH | CH | CCN | H |
| 150 | 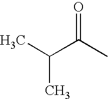 | Cl | CH | F | CH | CCN | H |
| 151 | 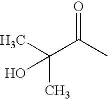 | Cl | CH | F | CH | CCN | H |
| 152 | 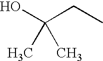 | Cl | CH | F | CH | CCN | H |
| 153 | 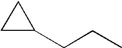 | 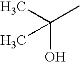 | CH | CH | CH | CCN | H |
| 154 | 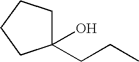 | Cl | CH | CH | CH | CCN | H |
| 155 | 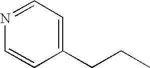 | Cl | CH | CH | CH | CCN | H |
| 156 | Br | 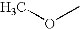 | CH | CH | CH | CCN | H |
| 157 | 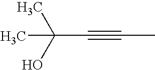 | 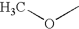 | CH | CH | CH | CCN | H |
| 158 | 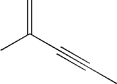 | Cl | CH | CH | CH | CCN | H |
| 159 | 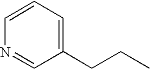 | 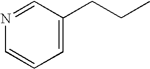 | CH | CH | CH | CCN | H |
| 160 | 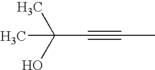 | 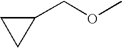 | CH | CH | CH | CCN | H |
| 161 | 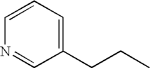 | 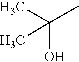 | CH | CH | CH | CCN | H |
| 162 | 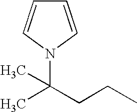 | Cl | CH | CH | CH | CCN | H |
| 163 | 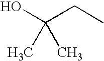 | 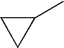 | CH | CH | CH | CCN | H |
| 164 | 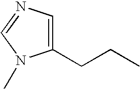 | Cl | CH | CH | CH | CCN | H |
| 165 | 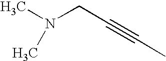 | Cl | CH | CH | CH | CCN | H |
| 166 | 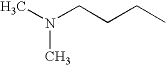 | Cl | CH | CH | CH | CCN | H |
| 167 | 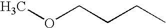 | Cl | CH | CH | CH | CCN | H |
| 168 | 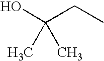 | 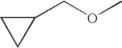 | CH | CH | CH | CCN | H |
| 169 | 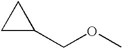 | 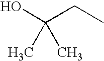 | CH | F | CH | CCN | H |
| 170 | 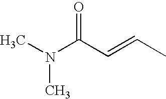 | Cl | CH | CH | CH | CCN | H |
| 171 | 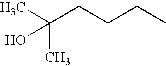 | Cl | CH | CH | CH | CCN | H |
| 172 | 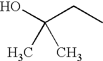 | 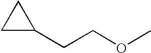 | CH | F | CH | CCN | H |
| 173 | Br | 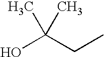 | CH | CH | CH | CCN | H |
| 174 | 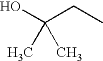 | 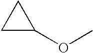 | CH | CH | CH | CCN | H |
| 175 | 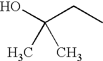 | 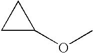 | CH | F | CH | CCN | H |
| 176 | 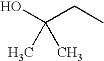 | 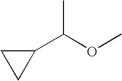 | CH | CH | CH | CCN | H |
| 177 | 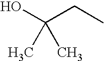 | 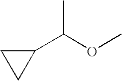 | CH | F | CH | CCN | H |
| 178 | OH | Cl | CH | CH | CH | CCN | H |
| 179 | Cl | 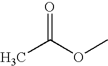 | CH | CH | CH | CCN | H |
| 180 | 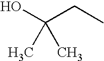 | 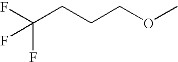 | CH | CH | CH | CCN | H |
| 181 | Cl | 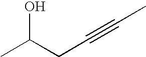 | CH | CH | CH | CCN | H |
| 182 | 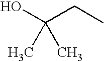 | 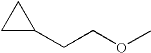 | CH | CH | CH | CCN | H |
| 183 | 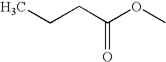 | Cl | CH | CH | CH | CCN | H |
| 184 | Cl | 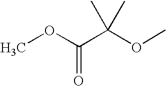 | CH | CH | CH | CCN | H |
| 185 | Cl | 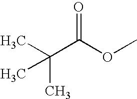 | CH | CH | CH | CCN | H |
| 186 | 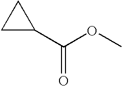 | Cl | CH | CH | CH | CCN | H |
| 187 | Br | Cl | CH | 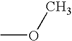 | CH | CCN | H |
| 188 | 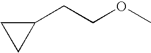 | 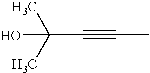 | CH | CF | CH | CCN | H |
| 189 | Cl | Br | CH | N | CH | CCN | H |
| 190 | 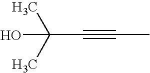 | Cl | CH | N | CH | CCN | H |

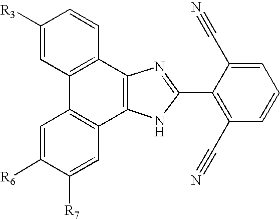

| EX | R3 | R6 | R7 |
|---|---|---|---|
| 191 | 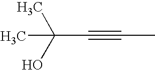 | Cl | 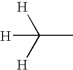 |
| 192 | Cl | H | Br |
| 193 | Cl | H | 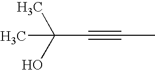 |
| 194 | Cl | H | 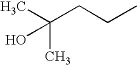 |

or a pharmaceutically acceptable salt of any of the above compounds.

In another embodiment, the invention encompasses the above methods wherein the compound that inhibits microsomal prostaglandin E synthase-1 is a third sub-class within the fifth class represented by Formula A wherein: R³ and R⁶ are independently selected from the group consisting of: hydrogen, fluoro, chloro, bromo, Iodo, cyano, methyl, methoxy, ethyl, vinyl, cyclopropyl, propyl, butyl, —CO₂i-Pr, —CO₂CH₃, —SO₂CF₃, 3-pyridyl, acetyl,

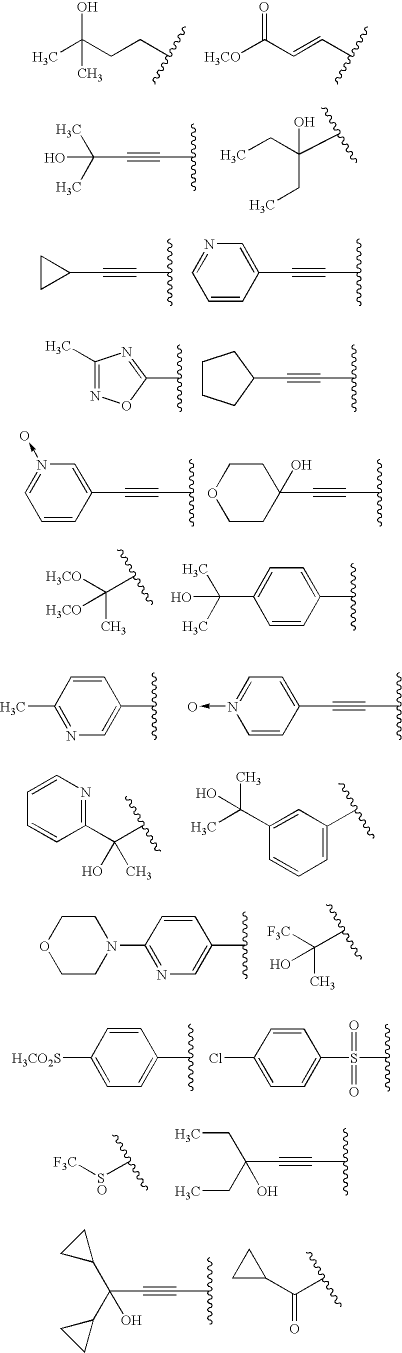

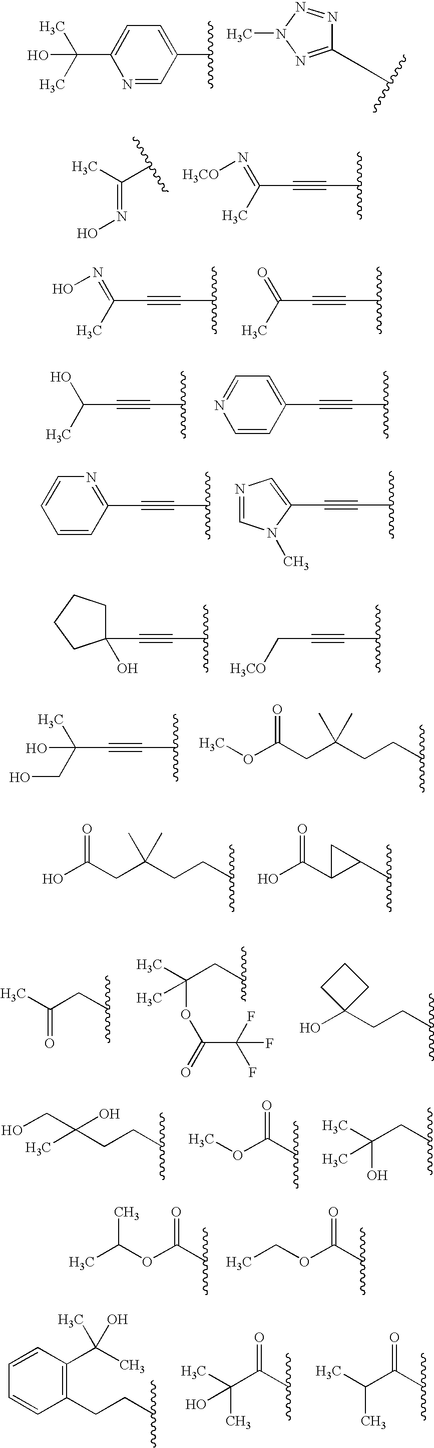

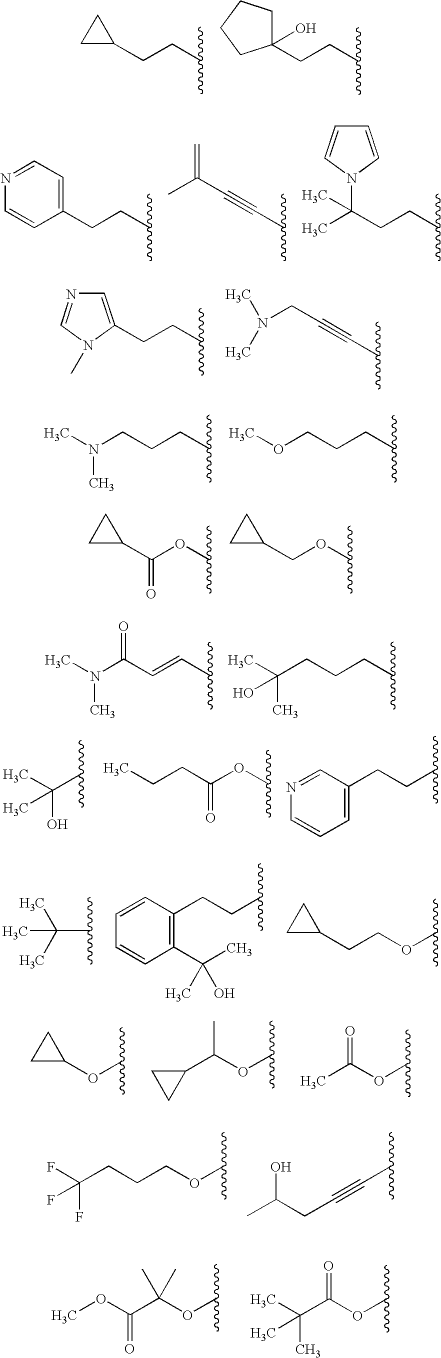

with the proviso that at least one of R³ or R⁶ is other than hydrogen.

In another embodiment, the invention encompasses the above methods wherein the compound that inhibits microsomal prostaglandin E synthase-1 is a third-sub-genus within the genus represented by Formula B:

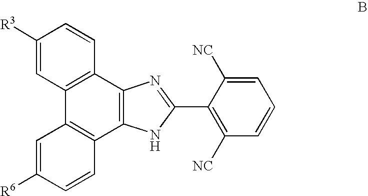

or a prodrug thereof, or a pharmaceutically acceptable salt of said compound or prodrug, wherein:

### R³ is

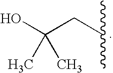

In another embodiment, the invention encompasses the above methods wherein the compound that inhibits microsomal prostaglandin E synthase-1 is first class within the third sub-genus represented by Formula B wherein R⁶ is R¹²—O.

In another embodiment, the invention encompasses the above methods wherein the compound that inhibits microsomal prostaglandin E synthase-1 is a sub-class within the first class represented by Formula B wherein R¹² is selected from the group consisting of: (1) C₁₋₄alkyl and (2) C₃₋₆cycloalkyl-C₁₋₄alkyl-, wherein said C₁₋₄alkyl and C₃₋₆cycloalkyl may each be optionally substituted with 1 to 3 substituents independently selected from the group consisting of: OH, F, Cl, Br and I.

In another embodiment, the invention encompasses the above methods wherein the compound that inhibits microsomal prostaglandin E synthase-1 is a second class within the third sub-genus represented by Formula B wherein R⁶ is selected from F, Cl, Br and I.

The term “treatment” includes partial or total inhibition of the neoplasia growth, spreading or metastasis, as well as partial or total destruction of the neoplastic cells. The term “prevention” includes either preventing the onset of clinically evident neoplasia altogether or preventing the onset of a preclinically evident stage of neoplasia in individuals at risk. Also intended to be encompassed by this definition is the prevention of initiation for malignant cells or to arrest or reverse the progression of premalignant cells to malignant cells. This includes prophylactic treatment of those at risk of developing the neoplasia. The term “subject” for purposes of treatment includes any human or mammal subject who has any one of the known neoplasias, and preferably is a human subject. For methods of prevention, the subject is any human or animal subject, and preferably is a human subject who is at risk for obtaining a neoplasia. The subject may be at risk due to exposure to carcinogenic agents, being genetically predisposed to have the neoplasia, and the like.

The term “neoplasia” includes both benign and cancerous tumors, growths and polyps. “Neoplasia” includes both new and existing tumors, growths and polyps. Benign tumors, growths and polyps include squamous cell papilloma, basal cell tumor, transitional cell papilloma, adenoma, gastrinoma, cholangiocellular adenoma, hepatocellular adenoma, renal tubular adenoma, oncocytoma, glomus tumor, melanocytic nevus, fibroma, myxoma, lipoma, leiomyoma, rhabdomyoma, benign teratoma, meangioma, osteoma, chondroma and meningioma. Cancerous tumors, growth and polyps include squamous cell carcinoma, basal cell carcinoma, transitional cell carcinoma, adenocarcinoma, malignant gastrinoma, cholangiocelleular carcinoma, hepatocellular carcinoma, renal cell carcinoma, malignant melanoma, fibrosarcoma, myxosarcoma, liposarcoma, leimyosarcoma, rhabdomyosarcoma, malignant teratoma, hemangiosarcoma, Kaposi sarcoma, lymphangiosarcoma, osteosarcoma, chondrosarcoma, malignant meningioma, non-Hodgkin lymphoma, Hodgkin lymphoma and leukemia. For purposes of this specification, “neoplasia” includes brain cancer, bone cancer, epithelial cell-derived neoplasia (epithelial carcinoma), basal cell carcinoma, adenocarcinoma, gastrointestinal cancer such as lip cancer, mouth cancer, esophogeal cancer, small bowel cancer and stomach cancer, colon cancer, rectal cancer, liver cancer, bladder cancer, pancreas cancer, ovary cancer, cervical cancer, lung cancer, breast cancer and skin cancer, such as squamus cell and basal cell cancers, prostate cancer, renal cell carcinoma, and other known cancers that affect epithelial, mesenchymal or blood cells throughout the body. The invention includes benign and cancerous tumors, growths and polyps of the following cell types: squamous epithelium, basal cells, transitional epithelium, glandular epithelium, G cells, bile ducts epithelium, hepatocytes, tubules epithelium, melanocytes, fibrous connective tissue, cardiac skeleton, adipose tissue, smooth muscle, skeletal muscle, germ cells, blood vessels, lymphatic vessels, bone, cartilage, meninges, lymphoid cells and hematopoietic cells. The method can be used to treat subjects having adenomatous polyps, including those with familial adenomatous polyposis (FAP). Additionally, the method can be used to prevent polyps from forming in patients at risk of FAP. Preferably, the invention encompasses treating or preventing the following cancers: colorectal, esophagus stomach, breast, head and neck, skin, lung, liver, gall bladder, pancreas, bladder, endometrium cervix, prostate, thyroid and brain.

The compounds described herein include, as appropriate, pharmaceutically acceptable salts. For purposes of this specification, the heading “R³/R⁶” means that the substituent indicated in that column is substituted at the position represented by either R³ or R⁶. In the adjacent column, the heading “R⁶/R³” means the indicated substituent is substituted at the position R³ or R⁶ not substituted in the previous column. By way of example, Example 6 represents R³═CN and R⁶═H or R³═H and R⁶═CN, representing both tautomers.

The term “halogen” or “halo” includes F, Cl, Br, and I.

The term “alkyl” means linear or branched structures and combinations thereof, having the indicated number of carbon atoms. Thus, for example, C₁₋₆alkyl includes methyl, ethyl, propyl, 2-propyl, s- and t-butyl, butyl, pentyl, hexyl and 1,1-dimethylethyl.

The term “alkenyl” means linear or branched structures and combinations thereof, of the indicated number of carbon atoms, having at least one carbon-to-carbon double bond, wherein hydrogen may be replaced by an additional carbon-to-carbon double bond. C₂₋₆alkenyl, for example, includes ethenyl, propenyl, 1-methylethenyl, butenyl and the like.

The term “alkynyl” means linear or branched structures and combinations thereof, of the indicated number of carbon atoms, having at least one carbon-to-carbon triple bond. C₃₋₆alkynyl, for example, includes, propenyl, 1-methylethenyl, butenyl and the like.

The term “alkoxy” means alkoxy groups of a straight, branched or cyclic configuration having the indicated number of carbon atoms. C₁₋₆alkoxy, for example, includes methoxy, ethoxy, propoxy, isopropoxy, and the like.

The term “cycloalkyl” means mono-, bi- or tri-cyclic structures, optionally combined with linear or branched structures, having the indicated number of carbon atoms. Examples of cycloalkyl groups include cyclopropyl, cyclopentyl, cycloheptyl, adamantyl, cyclododecylmethyl, 2-ethyl-1-bicyclo[4.4.0]decyl, cyclobutylmethyl cyclopropylmethyl and the like.

Compounds described herein may contain an asymmetric center and may thus exist as enantiomers. Where the compounds according to the invention possess two or more asymmetric centers, they may additionally exist as diastereomers. The present invention includes all such possible stereoisomers as substantially pure resolved enantiomers, racemic mixtures thereof, as well as mixtures of diastereomers. The above Formula I is shown without a definitive stereochemistry at certain positions. The present invention includes all stereoisomers of Formula I and pharmaceutically acceptable salts thereof. Diastereoisomeric pairs of enantiomers may be separated by, for example, fractional crystallization from a suitable solvent, and the pair of enantiomers thus obtained may be separated into individual stereoisomers by conventional means, for example by the use of an optically active acid or base as a resolving agent or on a chiral HPLC column. Further, any enantiomer or diastereomer of a compound of the general Formula I may be obtained by stereospecific synthesis using optically pure starting materials or reagents of known configuration.

Some of the compounds described herein contain olefinic double bonds, and unless specified otherwise, are meant to include both E and Z geometric isomers.

Some of the compounds described herein may exist with different points of attachment of hydrogen, referred to as tautomers. The compound of Formula I exists in the following tautomeric forms:

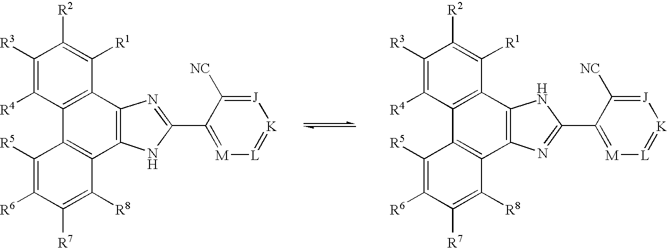

The individual tautomers as well as mixture thereof are encompassed within Formula I.

The compounds described herein include within its scope prodrugs of the compounds of this invention. In general, such prodrugs will be functional derivatives of the compounds of this invention which are readily convertible in vivo into the required compound. Thus, in the methods of treatment of the present invention, the term “administering” shall encompass the treatment of the various conditions described with the compound specifically disclosed or with a compound which may not be specifically disclosed, but which converts to the specified compound in vivo after administration to the patient. Conventional procedures for the selection and preparation of suitable prodrug derivatives are described, for example, in “Design of Prodrugs,” ed. H. Bundgaard, Elsevier, 1985. Metabolites of these compounds include active species produced upon introduction of compounds of this invention into the biological milieu. Exemplifying prodrugs of the invention are compounds of Formula C.

The term “amounts that are effective to treat” is intended to mean that amount of a drug or pharmaceutical agent that will elicit the biological or medical response of a tissue, a system, animal or human that is being sought by a researcher, veterinarian, medical doctor or other clinician. The term also encompasses the amount of a pharmaceutical drug that will prevent or reduce the risk of occurrence of the biological or medical event that is sought to be prevented in a tissue, a system, animal or human by a researcher, veterinarian, medical doctor or other clinician. Suitable dosage levels of the compound of Formula I used in the present invention ate described below. The compound may be administered on a regimen of once or twice per day.

The compounds described herein may be administered as a pharmaceutical composition of comprising a compound of Formula I as an active ingredient or a pharmaceutically acceptable salt, thereof, and may also contain a pharmaceutically acceptable carrier and optionally other therapeutic ingredients. The term “pharmaceutically acceptable salts” include salts prepared from bases that result in non-toxic pharmaceutically acceptable salts, including inorganic bases and organic bases. Salts derived from inorganic bases include aluminum, ammonium, calcium, copper, ferric, ferrous, lithium, magnesium, manganic salts, manganous, potassium, sodium, zinc, and the like. Particularly preferred are the ammonium, calcium, magnesium, potassium, and sodium salts. Salts derived from pharmaceutically acceptable organic non-toxic bases include salts of primary, secondary, and tertiary amines, substituted amines including naturally occurring substituted amines, cyclic amines, and basic ion exchange resins, such as arginine, betaine, caffeine, choline, N,N-dibenzylethylenediamine, diethyl amine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine, and the like.

When the compounds described herein are basic, salts may be prepared from acids that result in pharmaceutically acceptable salts, including inorganic and organic acids. Such acids include acetic, adipic, aspartic, 1,5-naphthalenedisulfonic, benzenesulfonic, benzoic, camphorsulfonic, citric, 1,2-ethanedisulfonic, ethanesulfonic, ethylenediaminetetraacetic, fumaric, glucoheptonic, gluconic, glutamic, hydriodic, hydrobromic, hydrochloric, isethionic, lactic, maleic, malic, mandelic, methanesulfonic, mucic, 2-naphthalenesulfonic, nitric, oxalic, pamoic, pantothenic, phosphoric, pivalic, propionic, salicylic, stearic, succinic, sulfuric, tartaric, p-toluenesulfonic acid, undecanoic, 10-undecenoic, and the like.

The pharmaceutical compositions containing the active ingredient may be in a form suitable for oral use, for example, as tablets, troches, lozenges, aqueous or oily suspensions, dispersible powders or granules, emulsions, hard or soft capsules, or syrups or elixirs.

Compositions intended for oral use may be prepared according to any method known to the art for the manufacture of pharmaceutical compositions and such compositions may contain one or more agents selected from the group consisting of sweetening agents, flavoring agents, coloring agents and preserving agents in order to provide pharmaceutically elegant and palatable preparations. Tablets contain the active ingredient in admixture with non-toxic pharmaceutically acceptable excipients which are suitable for the manufacture of tablets. These excipients may be for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example, magnesium stearate, stearic acid or talc. The tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed. They may also be coated by the technique described in the U.S. Pat. Nos. 4,256,108; 4,166,452; and 4,265,874 to form osmotic therapeutic tablets for control release.

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredients is mixed with water or an oil medium, for example peanut oil, liquid paraffin, or olive oil. Exemplifying a formulation for the present invention is a dry filled capsule containing a 50/50 blend of microcrystalline cellulose and lactose and 1 mg, 10 mg or 100 mg of the compound of Formula I.

Aqueous suspensions contain the active material in admixture with excipients suitable for the manufacture of aqueous suspensions. Such excipients are suspending agents, for example sodium carboxymethyl-cellulose, methylcellulose, hydroxypropylmethyl-cellulose, sodium alginate, polyvinyl-pyrrolidone, gum tragacanth and gum acacia; dispersing or wetting agents may be a naturally-occurring phosphatide, for example lecithin, or condensation products of an alkylene oxide with fatty acids, for example polyoxyethylene stearate, or condensation products of ethylene oxide with long chain aliphatic alcohols, for example heptadecaethylene-oxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl, p-hydroxybenzoate, one or more coloring agents, one or more flavoring agents, and one or more sweetening agents, such as sucrose, saccharin or aspartame.

Liquid formulations include the use of self-emulsyfying drug delivery systems and NanoCrystal® technology. Cyclodextrin inclusion complexes can also be utilized.

Oily suspensions may be formulated by suspending the active ingredient in a vegetable oil, for example arachis oil, olive oil, sesame oil or coconut oil, or in mineral oil such as liquid paraffin. The oily suspensions may contain a thickening agent, for example beeswax, hard paraffin or cetyl alcohol. Sweetening agents such as those set forth above, and flavoring agents may be added to provide a palatable oral preparation. These compositions may be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, for example sweetening, flavoring and coloring agents, may also be present.

The pharmaceutical compositions of the invention may also be in the form of an oil-in-water emulsions. The oily phase may be a vegetable oil, for example olive oil or arachis oil, or a mineral oil, for example liquid paraffin or mixtures of these. Suitable emulsifying agents may be naturally-occurring phosphatides, for example soy bean, lecithin, and esters or partial esters derived from fatty acids and hexitol anhydrides, for example sorbitan monooleate, and condensation products of the said partial esters with ethylene oxide, for example polyoxyethylene sorbitan monooleate. The emulsions may also contain sweetening and flavouring agents.

Syrups and elixirs may be formulated with sweetening agents, for example glycerol, propylene glycol, sorbitol or sucrose. Such formulations may also contain a demulcent, a preservative and flavoring and coloring agents. The pharmaceutical compositions may be in the form of a sterile injectable aqueous or oleagenous suspension. This suspension may be formulated according to the known art using those suitable dispersing or wetting agents and suspending agents which have been mentioned above. The sterile injectable preparation may also be a sterile injectable solution or suspension in a non-toxic parenterally-acceptable diluent or solvent, for example as a solution in 1,3-butane diol. Among the acceptable vehicles and solvents that may be employed are water, Ringer's solution and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

Compounds of Formula I may also be administered in the form of suppositories for rectal administration of the drug. These compositions can be prepared by mixing the drug with a suitable non-irritating excipient which is solid at ordinary temperatures but liquid at the rectal temperature and will therefore melt in the rectum to release the drug. Such materials are cocoa butter and polyethylene glycols.

For topical use, creams, ointments, jellies, solutions or suspensions, etc., containing the compound of Formula I are employed. (For purposes of this application, topical application shall include mouth washes and gargles.)

Pharmaceutical compositions of the invention may also utilize absorption enhancers such as tween 80, tween 20, Vitamin E TPGS (d-alpha-tocopheryl polyethylene glycol 1000 succinate) and Gelucire®.

Dosage levels of the order of from about 0.01 mg to about 140 mg/kg of body weight per day are useful in the treatment of the above-indicated conditions, or alternatively about 0.5 mg to about 7 g per patient per day. For example, neoplasia may be effectively treated by the administration of from about 0.01 to 50 mg of the compound per kilogram of body weight per day, or alternatively about 0.5 mg to about 3.5 g per patient per day, preferably 2.5 mg to 1 g per patient per day.

The amount of active ingredient that may be combined with the carrier materials to produce a single dosage form will vary depending upon the host treated and the particular mode of administration. For example, a formulation intended for the oral administration of humans may contain from 0.5 mg to 5 g of active agent compounded with an appropriate and convenient amount of carrier material which may vary from about 5 to about 95 percent of the total composition. Dosage unit forms will generally contain between from about 1 mg to about 500 mg of an active ingredient, typically 25 mg, 50 mg, 100 mg, 200 mg, 300 mg, 400 mg, 500 mg, 600 mg, 800 mg, or 1000 mg. Dosage amounts of 4 mg, 8 mg, 18 mg, 20 mg, 36 mg, 40 mg, 80 mg, 160 mg, 320 mg and 640 mg may also be employed. Dosage unit forms containing 1, 10 or 100 mg are also encompassed.

It will be understood, however, that the specific dose level for any particular patient will depend upon a variety of factors including the age, body weight, general health, sex, diet, time of administration, route of administration, rate of excretion, drug combination and the severity of the particular disease undergoing therapy.

### Methods of Synthesis

The compounds of Formula I of the present invention can be prepared according to the synthetic routes outlined in Schemes 1 and 4 below and by following the methods described therein. The imidazole of Formula I may be prepared in a multi-step sequence from the requisite phenanthrenequinone i. The phenanthrene imidazole iii is obtained by treating the phenanthrenequinone i and an appropriately substituted aldehyde ii with a reagent such as NH₄OAc or NH₄HCO₃ in a solvent such as acetic acid. Treatment of the imidazole iii with CuCN in a solvent such as DMF or DMSO produces the mono or bis-nitrile (M=CCN) Ia. Subsequent functional group interconversion can be done at any of the R¹ to R⁸ positions. For example, if one or more of the R¹ to R⁸ substituents equal Cl, Br or I and if M is different from CBr or Cl, Ia could be converted to Ib by placing Ia in the presence of a monosubstituted alkynyl, a stannane, a boronic acid, a borane or a boronate under conditions that promote cross coupling reaction, such as heating in the presence of a catalyst, such as Pd(PPh₃)₄ and CuI, in the presence of a base, such as sodium carbonate or diisopropylamine, and in an suitable solvent, such as THF, DMF or DME. This last exemplified step, or any other appropriate functional group transformation, can be iteratively repeated on R¹ to R⁸.

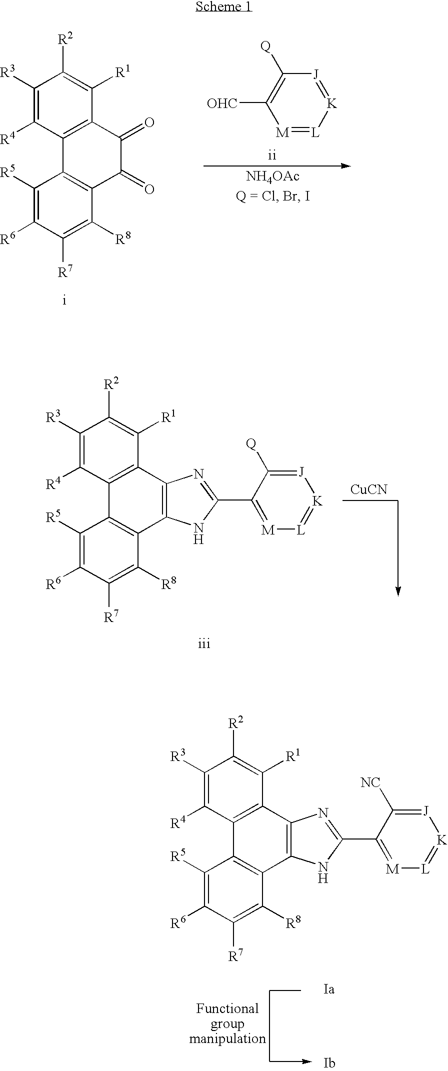

Phenanthrenequinone i can be prepared according to the sequences outlined in Scheme 2 and 3. Deprotonation of the phosphonium salt iv (Scheme 2) in the presence of a base, such as sodium hydride or sodium methoxide, in a solvent such as DMF followed by the addition of the aldehyde v produces the stylbene vi as a mixture of E and Z isomers. Intramolecular cyclisation of this mixture upon exposition to UV light in the presence of an oxidizing agent, such as iodine, and an acid scavenger, such as propylene oxide, in a suitable solvent such as cyclohexanne produces the phenanthrene vii. This phenanthrene viia can be directly oxidized with an oxidizing agent, such as CrO3, in a suitable solvent, such as acetic acid, to provide the phenanthrenequinone i, or optionally, phenanthrene viia could be further elaborated to phenanthrene viib by the appropriate interconversion of any of the functional group R¹ to R⁸, such as transmetallation with an organometallic reagent, such as butyl lithium, in a suitable solvent such as THF, followed by the addition of an electrophile, such as iodine or carbon dioxide. Alternatively (Scheme 3), phenylacetic acid viii can be condensed with the aldehyde ix in the presence of a base, such as potassium carbonate, and in the presence of acetic anhydride to afford the nitro stylbene x. This nitro aryl x is then reduced with an appropriate reducing agent, such as iron or iron sulfate, in the presence of ammonium hydroxide in a suitable solvent, such as acetic acid, to produce the amine xi. Diazotization of this amine xi with sodium nitrite in the presence of aqueous hydroxide, such as sodium hydroxide, followed by acidification with an acid, such as sulfuric acid and sulfamic acid, and cyclization in the presence of a catalyst, such as copper or a ferrocene, generates the phenanthrene carboxylic acid xii. This phenanthrene can be oxidized and simultaneously decarboxylated using an appropriate oxidizing agent, such as chromium trioxide in suitable solvent, such as acetic acid, to afford the phenanthrenequinone i.

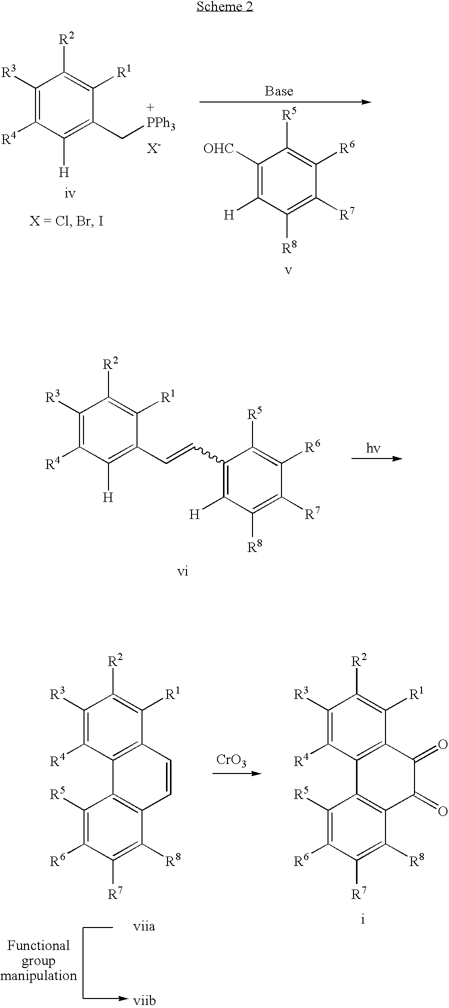

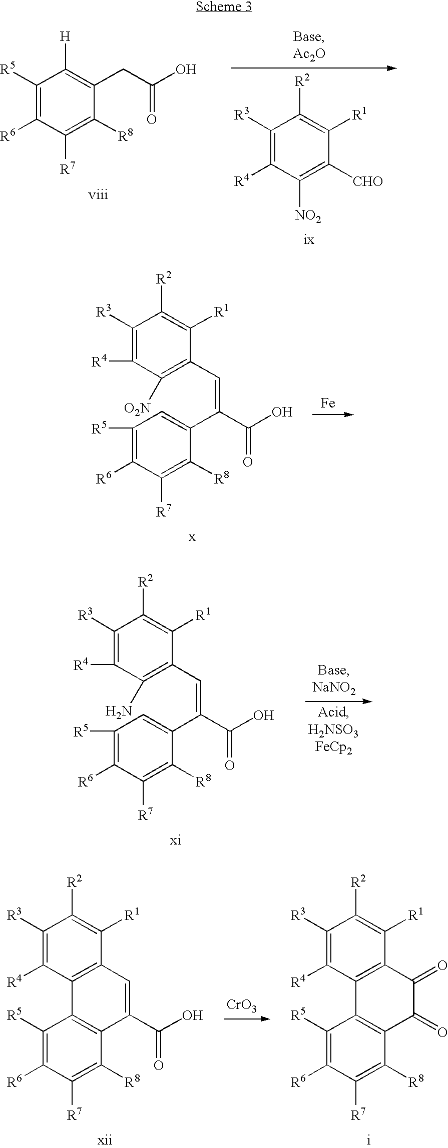

As shown in Scheme 4, protection of the halophenanthrene xiii with an appropriate protecting group such as 2-(trimethylsily)ethoxymethyl in the presence of a base, such as sodium hydride or diisopropylethylamine, in a suitable solvent, such as DMF or methylene chloride, affords the protected phenanthrene imidazole xiv. This phenanthrene imidazole xiv is then carbonylated with carbon monoxide in the presence of a catalyst, such as Pd(OAc)₂, and in the presence of a base, such as triethylamine, in a mixture of an alcoholic solvent, such as methanol and DMF, or any other suitable organic solvent. Treatment of the ester xv with a nucleophilic reagent such as an organolithium, organocerium or Grignard reagent in an organic solvent, such as ether, THF or methylene chloride (Grinard reagent), provides the tertiary alcohol xvi. Removal of the imidazole protecting group, for example by treating xvi with a mineral acid such as hydrochloric acid or in the presence of a fluoride source such as TBAF, in an organic solvent such as THF, affords the unprotected imidazole xvii. Treatment of this phenanthrene imidazole xvii with CuCN in a solvent, such as DMF or DMSO, produced the mono or bis-nitrile (M=CCN) Id. Subsequent functional group interconversion can be done at any of the R¹ to R⁸ positions. For example, if one or more of the R¹ to R⁸ substituents equal Cl, Br or I and if M is different from CBr or Cl, Id could be converted to Ie by placing Id in the presence of a monosubstituted alkynyl, a stannane, a boronic acid, a borane or a boronate under conditions that promote cross coupling reaction, such as heating in the presence of a catalyst such as Pd(PPh₃)₄ and CuI, and in the presence of a base, such as sodium carbonate or diisopropylamine, in a suitable solvent, such as THF, DMF or DME. This last exemplified step, or any other appropriate functional group transformation, can be iteratively repeated on R¹ to R⁸.

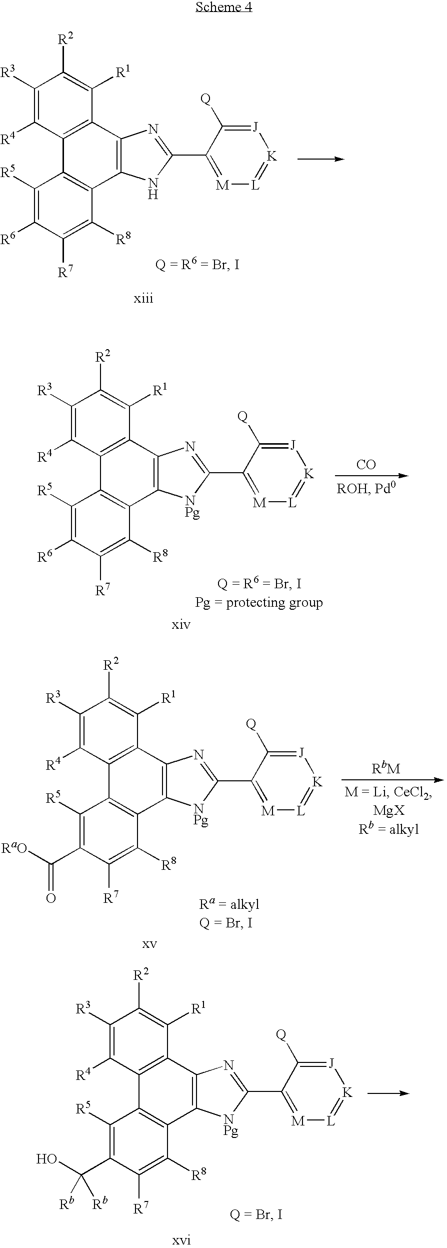

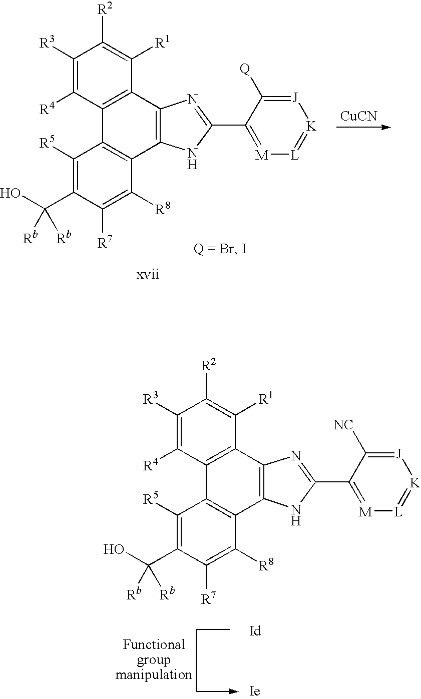

The imidazole secondary amine can be substituted as described in Scheme 5 by treating an appropriately functionalized phenanthrene imidazole I with a reagent such as an acylating agent or an alkylating agent such as methyl iodide in the presence of a base such as sodium hydride in a suitable solvent such as DMF.

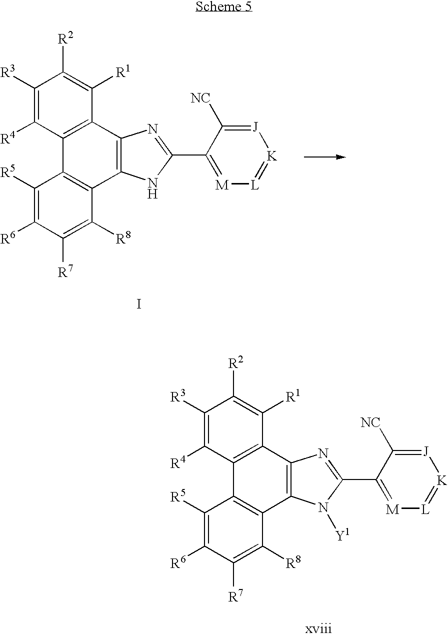

### EXAMPLES

The invention is exemplified by the following non-limiting examples:

### Example 14

### 2-[9-chloro-6-(3-hydroxy-3-methylbutyl-1-yn-1-yl)-1H-phenanthro[9,10-d]imidazol-2-yl]-3-fluorobenzonitrile

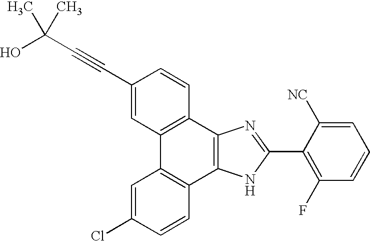

### Step 1: 6,9-dibromo-2-(2-chloro-6-fluorophenyl)-1H-phenanthro[9,10-d]imidazole

To a solution of 30 g (82 mmol) of 3,6-dibromophenanthrene-9,10-dione (Bhatt, Tetrahedron, 1963, 20, 803) in 1.0 L of acetic acid was added 25.9 g (328 mmol) of NH₄HCO₃ followed by 26 g (164 mmol) of 2-fluoro-6-chlorobenzaldehyde. The solution was stirred overnight at 130° C., cooled down to room temperature and poured into 2.5 L of water. The mixture was filtered, washed with water followed by hexane and diethyl ether. The resulting solid was refluxed in 1.0 L of toluene with a Dean-stark apparatus and approx. 100 mL of water was removed over 3 hrs. Upon cooling down to room temperature, a beige solid crystallized out of solution. This solid was filtered, washed with toluene and pumped under reduced pressure to afford 32 g (80%) of 6,9-dibromo-2-(2-chloro-6-fluorophenyl)-1H-phenanthro[9,10-d]imidazole.

### Step 2: 2-(6-bromo-9-chloro-1H-phenanthro[9,10-d]imidazol-2-yl)-3-fluorobenzonitrile

To a DMF (10 mL) solution of 3.0 g 6,9-dibromo-2-(2-chloro-6-fluorophenyl)-1H-phenanthro[9,10-d]imidazole from Step 1, was added 587 mg of CuCN and the solution was stirred overnight at 130° C. The solution was cooled down to room temperature followed by the addition of aqueous ammonium hydroxide and ethyl acetate. Layers were separated and the organic layer was washed with brine, dried over sodium sulphate and volatiles were removed under reduced pressure. The residue was purified by flash chromatography on silica gel using a gradient of 30% to 50% ethyl acetate/hexane to afford 500 mg of 2-(6-bromo-9-chloro-1H-phenanthro[9,10-d]imidazol-2-yl)-3-fluorobenzonitrile.

### Step 3: 2-[9-chloro-6-(3-hydroxy-3-methylbutyl-1-yn-1-yl)-1H-phenanthro[9,10-d]imidazol-2-yl]-3-fluorobenzonitrile

To a DMF (2 mL) solution of 2-(6-bromo-9-chloro-1H-phenanthro[9,10-d]imidazol-2-yl)-3-fluorobenzonitrile (320 mg) from Step 2 was added 5 mL of triethylamine, 0.1 mL of 2-methyl-3-butyn-2-ol, 20 mg of CuI and 82 mg of Pd(PPh₃)₄. The resulting mixture was stirred overnight at 80° C., cooled down to room temperature and diluted with ethyl acetate/water. The organic layer was washed with brine, dried over sodium sulphate and the volatiles were removed under reduced pressure. The residue was purified by flash chromatography on silica gel using a gradient of 30% to 50% ethyl acetate/hexane to afford 85 mg of 2-[9-chloro-6-(3-hydroxy-3-methylbutyl-1-yn-1-yl)-1H-phenanthro[9,10-d]imidazol-2-yl]-3-fluorobenzonitrile. ¹H NMR (Acetone-d₆): δ 8.89 (s, 2H), 8.71 (bs, 1H), 8.51 (bs, 1H), 7.93 (d, 1H), 8.88-8.72 (m, 4H), 4.55 (s, 1H), 1.65 (s, 6H).

### Example 25

### 2-(6-chloro-1H-phenanthro[9,10-d]imidazol-2-yl)isophthalonitrile

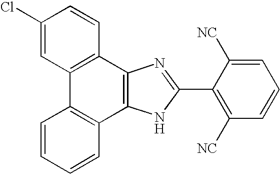

### Step 1: 1-(3-phenanthryl)ethanone oxime

In 200 mL of absolute ethanol was combined a mixture of 50 g (0.23 mol) of 1-(3-phenanthryl)ethanone and 40 g of hydroxylamine hydrochloride. The solution was heated to reflux followed by the addition of 70 mL of pyridine. After 3 hrs, the reaction was cooled down to room temperature and the solution rotovaped down. A mixture of ice/water was added to the residue and the mixture was stirred for 1 hr. The resulting off-white solid was filtered, washed with water and air dried to afford, after recristallization in diethyl ether, 32 g of 1-(3-phenanthryl)ethanone oxime.

### Step 2: 3-phenanthrylamine

To 385 g of polyphosphoric acic at 100° C. was added 32 g (0.14 mol) of 1-(3-phenanthryl)ethanone oxime from Step 1 over 30 minutes. The mixture was stirred at 100° C. for 2 hrs, cooled down to room temperature followed by the addition of water/ice. Stirred 30 minutes, filtered and washed with water. This white solid was then placed in 500 mL of methanol and 40 mL of concentrated HCl. The reaction was refluxed overnight, cooled down to room temperature and concentrated down. A mixture of ethyl acetate/water was added to the residue and the resulting solution was made basic with 10 N KOH. The aqueous layer was extracted with ethyl acetate and combined organic layers were washed with water, brine, dried over sodium sulphate and volatiles were removed under reduced pressure to afford 25 g of 3-phenanthrylamine as a beige solid.

### Step 3: 3-chlorophenanthrene

CuCl₂ (21 g) was dried under high vacuum at 115° C. for 90 minutes then cooled down to 65° C. followed by the addition of 250 mL of dry acetonitrile and 26 g of t-butyl nitrite. The 3-phenanthrylamine (25 g) from Step 2 was added over 30 minutes as a solution in 100 mL of acetonitrile. The reaction was stirred 45 minutes at 65° C., cooled down to room temperature followed by the addition of 1 L of 1 N HCl. The aqueous layer was extracted with methylene chloride and combined organic layers were washed with water, brine, dried over sodium sulphate and volatiles were removed under reduced pressure. The residue was purified by flash chromatography on silica gel using hexane as the eluent to afford a white solid which was recrystallized from hexane to produce 14.4 g of 3-chlorophenanthrene as a white solid.

### Step 4: 3-chlorophenanthrene-9,10-dione

To a solution of 12.5 g (58.7 mmol) of 3-chlorophenanthrene from Step 3 in 350 mL of acetic acid was added 23.5 g (0.23 mol) of CrO3. The reaction was stirred 2 hrs at 100° C., cooled down to room temperature and poured into 2 L of water. The suspension was stirred 1 hr, filtered and washed with water. The residue was dried under high vacuum to afford 12.5 g (88%) of 3-chlorophenanthrene-9,10-dione.

### Step 5: 6-chloro-2-(2,6-dibromophenyl)-1H-phenanthro[9,10-d]imidazole

This imidazole was prepared by following the procedure describe in Example 14, Step 1, but substituting 3-chlorophenanthrene-9,10-dione for 3,6-dibromophenanthrene-9,10-dione and substituting 2,6-dibromobenzaldehyde for 2-fluoro-6-chlorobenzaldehyde to afford 27 g of 6-chloro-2-(2,6-dibromophenyl)-1H-phenanthro[9,10-d]imidazole as an off-white solid.

### Step 6: 2-(6-chloro-1H-phenanthro[9,10-d]imidazol-2-yl)isophthalonitrile

To a DMF (300 mL) solution of 32 g (65.7 mmol) of 6-chloro-2-(2,6-dibromophenyl)-1H-phenanthro[9,10-d]imidazole from Step 5 was added 14.7 g of CuCN. The reaction was stirred overnight at 80° C., cooled down to room temperature, poured into a mixture of 1.5 L of water, 1.5 L of ethyl acetate and 200 mL of concentrated ammonium hydroxide and stirred 1 hr at room temperature. The aqueous layer was extracted with ethyl acetate and the combined organic layers were washed with 10% ammonium hydroxide, water, brine, dried over sodium sulphate and volatiles were removed under reduced pressure. The residue was swished in toluene (2×200 mL) and ethyl acetate (1 L). The obtained solid was purified by flash chromatography on silica gel in 5 portions using a gradient of 60% to 80% to 100% of ethyl acetate/hexane to afford 19.9 g of 2-(6-chloro-1H-phenanthro[9,10-d]imidazol-2-yl)isophthalonitrile as a pale yellow solid. ¹H NMR (400 MHz, DMSO): δ 14.32 (s, 1H), 9.0-8.9 (m, 2H), 8.55-8.45 (m, 4H), 7.99 (t, 1H), 7.85-7.78 (m, 2H), 7.72 (t, 1H).

### Example 36

### 2-(6-bromo-9-chloro-1H-phenanthro[9,10-d]imidazol-2-yl)isophthalonitrile

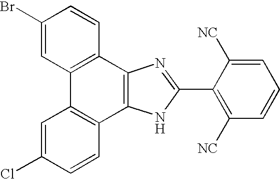

### Step 1: 1-bromo-4-[2-(4-chlorophenyl)vinyl]benzene

To a solution of (4-bromobenzyl)triphenylphosphonium bromide (396 g; 0.77 mol) in 2.5 L of DMF at 0° C., was added 37 g (0.92 mol) of NaH (60% in oil) in four portions. The solution was stirred 1 hr at 0° C. followed by the addition of 109 g (0.77 mol) of 4-chlorobenzaldehyde in two portions. This mixture was warmed up to room temperature, stirred 1 hr and quench by pouring the reaction into a 5° C. mixture of 10 L of water and 2.5 L of Et₂O. Aqueous layer was extracted with Et₂O, combined organic layers were washed with brine and dried over Na₂SO₄. Volatiles were removed under reduced pressure and the residue was dissolved in 1.5 L of cyclohexane and filtered through a pad of silica gel (wash with cyclohexane). 16 g of one isomer crystallized out of the solution as a white solid and after evaporation of the volatiles, 166 g of the other isomer 1-bromo-4-[2-(4-chlorophenyl)vinyl]benzene was isolated.

### Step 2: 3-bromo-6-chlorophenanthrene

A 2 L vessel equipped with a pyrex inner water-cooled jacket was charged with 5.16 g (17 mmol) of 1-bromo-4-[2-(4-chlorophenyl)vinyl]benzene from Step 1, 2 L of cyclohexane, 25 mL of THF, 25 mL of propylene oxide and 6.7 g (26 mmol) of iodine. The stirring solution was degassed by bubbling nitrogen and was exposed to UV light for 24 hrs by inserting a 450 W medium pressure mercury lamp in the inner. The reaction was quenched with 10% Na₂S₂O₃ and aqueous layer was extracted with ethyl acetate. Combined organic layers were washed with brine, dried over Na₂SO₄ and volatiles were removed under reduced pressure. The residue was swished in a minimal amount of ethyl acetate to afford approx. 5 g of 3-bromo-6-chlorophenanthrene as a solid.

### Step 3: 3-Bromo-6-chlorophenanthrene-9,10-dione

To a solution of 3-bromo-6-chlorophenanthrene from Step 2 (1.71 g; 5.86 mmol) in 35 mL of acetic acid was added 2.3 g (23.5 mmol) of CrO3. The mixture was stirred 2 hrs at 100° C., cooled down to room temperature, poured into 300 mL of water and stirred for 1 hr. The suspension was filtered, washed with water and Et₂O and pumped under reduced pressure to afford 1.67 g of 3-bromo-6-chlorophenanthrene-9,10-dione as a solid.

### Step 4: 9-bromo-6-chloro-2-(2,6-dibromophenyl)-1H-phenanthro[9,10-d]imidazole

To a solution of 15.5 g of 3-bromo-6-chlorophenanthrene-9,10-dione from Step 3 in 400 mL of acetic acid, was added 74.2 g of ammonium acetate and 19.1 g of 2,6-dibromobenzaldehyde. The mixture was stirred overnight at 120° C., cooled down to room temperature diluted in 4 L of water and filtered. The resulting solid was refluxed 2 hrs in toluene with a Dean Stark apparatus. After cooling down to room temperature, the suspension was filtered, the solid washed with toluene and the resulting beige solid dried under high vacuum to produce 26 g of 9-bromo-6-chloro-2-(2,6-dibromophenyl)-1H-phenanthro[9,10-d]imidazole.

### Step 5: 2-(9-bromo-6-chloro-1H-phenanthro[9,10-d]imidazol-2-yl)isophthalonitrile

To a solution of 26 g of 9-bromo-6-chloro-2-(2,6-dibromophenyl)-1H-phenanthro[9,10-d]imidazole from Step 4 in 200 mL of dry DMF, was added 14.2 g of CuCN. The reaction was stirred overnight at 85° C., cooled down to room temperature, brine was added and the mixture stirred for 30 minutes. The solution was diluted in ethyl acetate, washed with 10% ammonium hydroxide, brine, dried over sodium sulphate and volatiles were removed under reduced pressure to afford 26 g of 2-(9-bromo-6-chloro-1H-phenanthro[9,10-d]imidazol-2-yl)isophthalonitrile as a solid. ¹H NMR (Acetone-d₆): 9.19 (s, 1H), 9.02 (s, 1H), 9.71 (bs, 1H), 8.49 (bs, 1H), 8.39 (d, 2H), 8.07 (t, 1H), 7.97 (d, 1H), 8.81 (d, 1H).

### Example 40

### 2-[9-chloro-6-(3-hydroxy-3-methylbut-1-yn-1-yl)-1H-phenanthro[9,10-d]imidazol-2-yl]isophthalonitrile

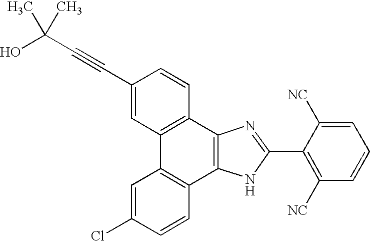

### Step 1: (2E)-2-(4-bromophenyl)-3-(4-chloro-2-nitrophenyl)acrylic acid

A 2 L flask equipped with a mechanical stirrer was charged with 183 g of 2-nitro-4-chlorobenzaldehyde, 212 g of 4-bromophenylacetic acid and 233 mL of acetic anhydride. To this solution was added 82 g of potassium carbonate and the reaction was stirred overnight at 100° C. The resulting dark mixture was cooled down to room temperature and 1.6 L of water was added followed by 800 mL of 10% HCl. The solution was decanted and taken up in water/ethyl acetate. Layers were separated, organic phase was washed with brine, dried over magnesium sulphate and volatiles were removed under reduced pressure. The residue was triturated in EtOH and the mother liquor was triturated 4 more times with EtOH to afford 219 g of the desired (2E)-2-(4-bromophenyl)-3-(4-chloro-2-nitrophenyl)acrylic acid.

### Step 2: (2E)-3-(2-amino-4-chlorophenyl)-2-(4-bromophenyl)acrylic acid

To a 50° C. solution of 135 g of (2E)-2-(4-bromophenyl)-3-(4-chloro-2-nitrophenyl)acrylic acid from Step 1 in 1.2 L of acetic acid and 80 mL of water, was added 98 g of iron (powder) portion wise maintaining the temperature below 50° C. The mixture was stirred 2 hrs at 50° C., cooled down to room temperature, diluted with ethyl acetate (1 L) and filtered through a plug of celite. Water (1 L) was added, the layers were separated and the organic layer was washed 2 times with water, brine, dried over magnesium sulphate and volatiles were removed under reduced pressure. Residual acetic acid was removed by the addition of 1 L of H₂O to the crude mixture, the solution was filtered and washed with an additional 1 L of H₂O and finally the solid was dried under high vacuum to afford 130 g of (2E)-3-(2-amino-4-chlorophenyl)-2-(4-bromophenyl)acrylic acid.

### Step 3: 3-Bromo-6-chlorophenanthrene-9,10-dione

This quinone can be obtained by following the procedure describe in Example 36, Step 1 to 3, or by the using the following procedure: to a 0° C. solution of 118 mL of concentrated sulphuric acid in 1.0 L of water was added drop wise a solution prepared as follows: 65 g of (2E)-3-(2-amino-4-chlorophenyl)-2-(4-bromophenyl)acrylic acid from Step 2 in 1 L of water followed by the addition of 11 g of NaOH, stirring for 10 minutes at 0° C., addition of NaNO₂ (15 g) and stirring of the resulting solution at 0° C. for 20 minutes. After 30 minutes, sulfamic acid (12.5 g) was added to this mixture and after the gaz evolution seized, 1.3 L of acetone was added and the solution was stirred at 0° C. for 10 minutes. This mixture was then added to a solution of ferrocene (6.9 g) in 480 mL of acetone resulting in the formation of a green precipitate. After stirring for 20 minutes, water (2.0 L) was added, the solid was filtered and the 6-bromo-3-chlorophenanthrene-9-carboxylic acid was obtained and allowed to air dry. This crude phenanthrene was placed in 2.0 L of acetic acid followed by the addition of 54 g of CrO3. The reaction was placed at 110° C. and after stirring for 1 hr, 18 g of CrO3 were added. The reaction was monitored by TLC and 18 g of CrO3 were added every hour for 3 hours where 100% conversion was observed by ¹H NMR. The mixture was cooled to room temperature, diluted in water (2.0 L), filtered and washed with water (1.0 L) to afford, after drying, 37 g of 3-Bromo-6-chlorophenanthrene-9,10-dione as a yellow solid.

### Step 4: 9-bromo-6-chloro-2-(2,6-dibromophenyl)-1H-phenanthro[9,10-d]imidazole

This imidazole was obtained following the procedure describe for Example 36, Step 4.

### Step 5: 2-(9-bromo-6-chloro-1H-phenanthro[9,10-d]imidazol-2-yl)isophthalonitrile

This imidazole was obtained following the procedure describe for Example 36, Step 5.

### Step 6: 2-[9-chloro-6-(3-hydroxy-3-methylbut-1-yn-1-yl)-1H-phenanthro[9,10-d]imidazol-2-yl]isophthalonitrile

To a solution of 13 g of 2-(9-bromo-6-chloro-1H-phenanthro[9,10-d]imidazol-2-yl)isophthalonitrile in 240 mL of DMF is added 5.5 mL of 2-methyl-3-butyn-2-ol, 2.0 g of tetrakis(triphenylphosphine)palladium, 1.1 g of copper iodide and 5.6 mL of diisopropylamine. The mixture is stirred at 55° C. for 1 hr then cooled to room temperature and diluted with ethyl acetate (250 mL). Water (250 mL) is added and the layers were separated, the organic phase is washed with brine, dried over magnesium sulphate and volatiles are removed under reduced pressure. The crude mixture is then purified on silica gel using 50% hexane/ethyl acetate. The product is then recrystallized in THF and triturated in hot ethyl acetate/ether mixture to afford 5.4 g of [9-chloro-6-(3-hydroxy-3-methylbut-1-yn-1-yl)-1H-phenanthro[9,10-d]imidazol-2-yl]isophthalonitrile as a light yellow solid. ¹H NMR (Acetone-d₆): 8.93 (s, 2H), 8.53 (m, 2H), 8.36 (d, 2H), 8.01 (t, 1H), 7.78 (d, 2H), 4.53 (s, 1H), 1.61 (s, 6H).

### Example 60

### 2-(1-{[dihydroxy(dioxido)phosphino]methyl}-1H-phenanthro[9,10-d]imidazol-2-yl)isophthalonitrile

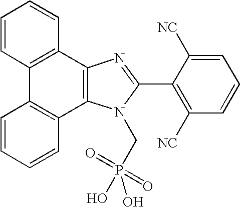

### Step 1: 2-(2,6-dibromophenyl)-1H-phenanthro[9,10-d]imidazole

This imidazole was obtained following the procedure described in Example 36, Step 4, but substituting the phenanthrene-9,10-dione for the 3-bromo-6-chlorophenanthrene-9,10-dione to afford the 2-(2,6-dibromophenyl)-1H-phenanthro[9,10-d]imidazole

### Step 2: 2-(1H-phenanthro[9,10-d]imidazol-2-yl)isophthalonitrile

This compound was obtained by using the procedure described in Example 36, Step 5, but substituting the 2-(2,6-dibromophenyl)-1H-phenanthro[9,10-d]imidazole for the 9-bromo-6-chloro-2-(2,6-dibromophenyl)-1H-phenanthro[9,10-d]imidazole to afford the desired 2-(1H-phenanthro[9,10-d]imidazol-2-yl)isophthalonitrile.

### Step 3: 2-[1-(chloromethyl)-1H-phenanthro[9,10-d]imidazol-2-yl]isophthalonitrile

2-(1H-phenanthro[9,10-d]imidazol-2-yl)isophthalonitrile from Step 2 (1 g, 2.91 mmol) was mixed with cesium carbonate (1.14 g, 3.49 mmol) in chloroiodomethane (10 mL). The mixture was heated to 80° C. overnight. The reaction was cooled to room temperature and poured into 200 mL water and 500 mL ethyl acetate. The layers were separated, and the organic layer was washed with 200 mL water, 200 mL saturated aqueous sodium bicarbonate solution, 100 mL brine, and dried over anhydrous magnesium sulfate. The solvent was removed under reduced pressure. The crude solid was purified by flash column chromatography using 40% ethyl acetate in hexane to give 357 mg of 2-[1-(chloromethyl)-1H-phenanthro[9,10-d]imidazol-2-yl]isophthalonitrile (31%) plus 650 mg of a mixture of product and starting material.

### Step 4: 2-(1-{[dihydroxy(dioxido)phosphino]methyl}-1H-phenanthro[9,10-d]imidazol-2-yl)isophthalonitrile

The 2-[1-(chloromethyl)-1H-phenanthro[9,10-d]imidazol-2-yl]isophthalonitrile from Step 3 (200 mg, 0.509 mmol) was mixed with tetramethylammonium di(tert-butyl)phosphate (288 mg, 1.02 mmol) in DMF (5 mL) and heated at 50° C. for 8 hours. It was cooled to room temperature and poured into 15 mL water and 35 mL ethyl acetate. The layers were separated, and the organic layer was washed with 10 mL water (twice), 10 mL saturated aqueous sodium bicarbonate solution, brine, and dried over anhydrous magnesium sulfate. The solvent was removed under reduced pressure. The crude solid was purified by flash column chromatography using 50-70% ethyl acetate in hexane to give 221 mg of protected phosphate (77%). 155 mg of this solid was dissolved in 10% TFA/toluene (3 mL) and stirred at room temperature overnight. The solvent was removed under reduced pressure. The resulting crude product was purified by a semi-preparative RP-HPLC using a C18 column and eluting with a gradient of 44-49% acetonitrile+0.2% TFA over 8 min. The fractions containing product were combined and lyophilized to give 80 mg of the desired 2-(1-{[dihydroxy(dioxido)phosphino]methyl}-1H-phenanthro[9,10-d]imidazol-2-yl)isophthalonitrile.

¹H NMR (DMSO): 9.05 (d, 1H), 8.95 (d, 1H), 8.54-8.61 (m, 2H), 8.47 (d, 2H), 8.06 (t, 1H), 8.70-8.85 (m, 4H), 6.21 (d, 2H).

### Example 87

### 2-[6-bromo-9-(1-hydroxy-1-methylethyl)-1H-phenanthro[9,10-d]imidazol-2-yl]isophthalonitrile

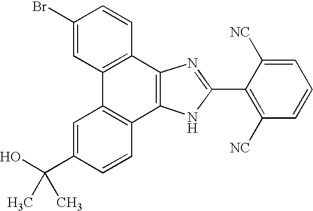

### Step 1: 6,9-dibromo-2-(2,6-dibromophenyl)-1H-phenanthro[9,10-d]imidazole

A suspension of di-bromoquinone (38.6 g, 0.1 mol), ammonium acetate (165 g, 2.1 mol) and dibromobenzaldehyde (45 g, 0.1 mol) in acetic acid (1.5 L) was heated at reflux for 16 h. The reaction mixture was quenched by pouring it into water (2.2 L), followed by stirring for 2 h. The resulting solid was filtered and rinsed successively with water and hexanes. The solids were then heated at reflux in toluene (600 mL) with a Dean Stark for 4 h and then filtered to afford the desired 6,9-dibromo-2-(2,6-dibromophenyl)-1H-phenanthro[9,10-d]imidazole as a beige powder (62.3 g, 97%).

### Step 2: 6,9-dibromo-2-(2,6-dibromophenyl)-1-{[2-(trimethylsilyl)ethoxy]methyl}-1H-phenanthro[9,10-d]imidazole

To a suspension of 6,9-dibromo-2-(2,6-dibromophenyl)-1H-phenanthro[9,10-d]imidazole from Step 1 (61.8 g, 0.1 mol) in THF (980 mL) at 0° C., was added sodium hydride (60% dispersion in mineral oil, 10 g, 0.25 mol). The suspension was stirred at 0° C. for 15 minutes, followed by addition of SEMCl (45 mL, 0.25 mol). The mixture was warmed to room temperature and stirred for 3 h, after which it was poured into water. The aqueous phase was extracted with ethyl acetate, the organic layer washed once with brine, dried over Na₂SO₄, filtered and concentrated. The crude material was swished in hexanes/diethyl ether for 4 h, then filtered to obtain 6,9-dibromo-2-(2,6-dibromophenyl)-1-{[2-(trimethylsilyl)ethoxy]methyl}-1H-phenanthro[9,10-d]imidazole as a beige powder (71.5 g, 95%).

### Step 3: methyl 6-bromo-2-(2,6-dibromophenyl)-1-{[2-(trimethylsilyl)ethoxy]methyl}-1H-phenanthro[9,10-d]imidazole-9-carboxylate

To a solution of 6,9-dibromo-2-(2,6-dibromophenyl)-1-{[2-(trimethylsilyl)ethoxy]methyl}-1H-phenanthro[9,10-d]imidazole from Step 2 (22.8 g, 30.8 mmol) in DMF (150 mL) and MeOH (150 mL) in a 3-necked 1 L round-bottomed flask, was added Pd(OAc)₂ (350 mg, 1.5 mmol) and dppf (1.7 g, 3.0 mmol). The mixture was degassed three times and back-filled with carbon monoxide. Triethylamine (9.5 mL, 43 mmol) was then added and the reaction mixture was heated at 60° C., under an atmosphere of carbon monoxide, for 1 h. The reaction was quenched by pouring it into water and ethyl acetate. It was then filtered through Celite, the aqueous phase extracted with ethyl acetate, the organic layer washed once with brine, dried over Na₂SO₄, filtered and concentrated. The crude material was purified by flash chromatography on silica (0-5% ethyl acetate in toluene) to afford the isomers of the desired methyl 6-bromo-2-(2,6-dibromophenyl)-1-{[2-(trimethylsilyl)ethoxy]methyl}-1H-phenanthro[9,10-d]imidazole-9-carboxylate as beige solids (9.8 g, 44%).

### Step 4: 2-[6-bromo-2-(2,6-dibromophenyl)-1H-phenanthro[9,10-d]imidazol-9-yl]propan-2-ol

To a −78° C. solution of isomeric methyl 6-bromo-2-(2,6-dibromophenyl)-1-{[2-(trimethylsilyl)ethoxy]methyl}-1H-phenanthro[9,10-d]imidazole-9-carboxylate from Step 3 (9.9 g, 13.8 mmol) in CH₂Cl₂ (200 mL) was added methyl magnesium bromide (3.0 M in Et₂O, 33 mL) via addition funnel. The mixture was then warmed to −40° C., stirred at this temperature for 0.5 h, then warmed to between −30 and −35° C. and stirred at this temperature for 2 h. The reaction mixture was then warmed to −25° C., stirred for 3 h, and then stirred at 0° C. for 1.5 h. The reaction was quenched by pouring it into water and ethyl acetate. The aqueous phase was extracted with ethyl acetate, the organic layer washed once with brine, dried over Na₂SO₄, filtered and concentrated. The crude product was dissolved in THF (150 mL) and cooled to 0° C. TBAF (1.0 M in THF, 35 mL) was then added and the mixture heated at reflux for 17 h, then quenched with 25% NH₄OAc, the aqueous phase extracted with ethyl acetate, the organic layer washed once with brine, dried over Na₂SO₄, filtered and concentrated. The material obtained after purification by flash chromatography on silica (5-30% THF in toluene) was swished in toluene for 5 h and then filtered to afford 2-[6-bromo-2-(2,6-dibromophenyl)-1H-phenanthro[9,10-d]imidazol-9-yl]propan-2-ol as a white powder (4.53 g, 56%, 2 steps).

### Step 5: 2-[6-bromo-9-(1-hydroxy-1-methylethyl)-1H-phenanthro[9,10-d]imidazol-2-yl]isophthalonitrile

Copper cyanide (420 mg, 4.7 mmol) was added to a room temperature solution of 2-[6-bromo-2-(2,6-dibromophenyl)-1H-phenanthro[9,10-d]imidazol-9-yl]propan-2-ol from Step 4 (1.25 g, 2.1 mmol) in DMF (100 mL) and the mixture heated at 80° C. for 18 h, after which it was poured into a mixture of NH₄OH and ethyl acetate and stirred for 1 h. The aqueous phase was extracted with ethyl acetate, the organic layer washed once with water, once with brine, dried over Na₂SO₄, filtered and concentrated. The material obtained after purification by flash chromatography on silica (20-80% ethyl acetate in toluene) was swished in ethyl acetate and THF for 2 h and then filtered to afford 2-[6-bromo-9-(1-hydroxy-1-methylethyl)-1H-phenanthro[9,10-d]imidazol-2-yl]isophthalonitrile as a yellow solid (250 mg, 25%).

¹H NMR δ (ppm) (DMSO with added TFA): 9.08 (1H, s), 8.90 (1H, s), 8.45-8.39 (4H, m), 7.99-7.91 (3H, m), 1.61 (6H, s).

### Example 88

### 2-[6-(cyclopropylethynyl)-9-(1-hydroxy-1-methylethyl)-1H-phenanthro[9,10-d]imidazol-2-yl]isophthalonitrile

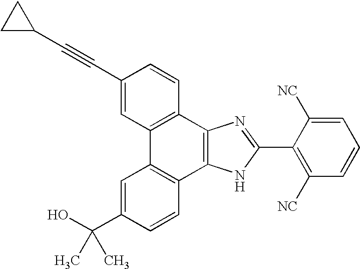

### Step 1: 2-[6-(cyclopropylethynyl)-9-(1-hydroxy-1-methylethyl)-1H-phenanthro[9,10-d]imidazol-2-yl]isophthalonitrile

A round bottomed flask containing 2-[6-bromo-9-(1-hydroxy-1-methylethyl)-1H-phenanthro[9,10-d]imidazol-2-yl]isophthalonitrile from Example 87 (1.26 g, 2.62 mmol), Pd(PPh₃)₄ (190 mg, 0.27 mmol) and copper iodide (100 mg, 0.52 mmol) was purged with nitrogen for 15 minutes, followed by addition of DMF (50 mL), cyclopropyl acetylene (1.4 mL, 21 mmol) and di-isopropylamine (560 μL, 4 mmol). The resulting mixture was heated at 60-65° C. for 3.5 h, cooled to room temperature and then poured into a mixture of NH₄OH and ethyl acetate and stirred for 1 h. The aqueous phase was extracted with ethyl acetate, the organic layer washed once with water, once with brine, dried over Na₂SO₄, filtered and concentrated. The material obtained after purification by flash chromatography on silica (30-100% ethyl acetate in toluene) was swished in toluene for 2 h and then filtered to afford 2-[6-(cyclopropylethynyl)-9-(1-hydroxy-1-methylethyl)-1H-phenanthro[9,10-d]imidazol-2-yl]isophthalonitrile as a yellow solid (350 mg). The mother liquor was combined with the mixed fractions and re-purified by flash chromatography on silica (3-40% acetonitrile in toluene) to afford 286 mg the bis-nitrile (total yield 52%).

1H NMR δ (ppm) (DMSO with added TFA): 8.92 (1H, s), 8.87 (1H, s), 8.43-8.39 (4H, m), 7.96 (1H, t), 7.90 (1H, d), 7.71 (1H, d), 1.60 (7H, s), 0.90 (2H, t), 0.84 (2H, d).

### Example 117

### 2-[9-chloro-6-(3-hydroxy-3-methylbutyl)-1H-phenanthro[9,10-d]imidazol-2-yl)isophthalonitrile

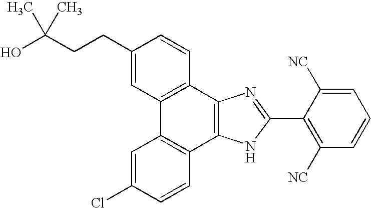

### Step 1: 2-[9-chloro-6-(3-hydroxy-3-methylbutyl)-1H-phenanthro[9,10-d]imidazol-2-yl)isophthalonitrile

To a solution of 9-BBN in THF (24 ml, 12 mmol, 0.5 M) was added 2-methyl-3-buten-2-ol (345 mg, 4.0 mmol) and the resulting solution was stirred under N₂ at rt for overnight. In a second flask charged with PdCl₂(dppf) (324 mg, 0.40 mmol), Cs₂CO₃ (2.4 g, 8.0 mmol) and Ph₃As (124 mg, 0.4 mmol) was added 2-(6-bromo-9-chloro-1H-phenanthro[9,10-d]imidazol-2-yl)isophthalonitrile from Example 36, DMF (24 ml) and H₂O (0.88 ml) and the mixture was stirred under N₂ for 5 minutes. The hydroboration mixture was then transferred to the second flask and the resulting reaction suspension was stirred at rt under N₂ for 5 days. After being treated with brine, the aqueous phase was extracted with EtOAc and the combined organic solution was washed with water and brine, dried over MgSO₄. After removing the drying agent by filtration, the solution was concentrated under reduced pressure and the residue was purified by silica gel chromatography (50% EtOAc/Hexane) to yield 600 mg of 2-[9-chloro-6-(3-hydroxy-3-methylbutyl)-1H-phenanthro[9,10-d]imidazol-2-yl)isophthalonitrile as a yellow solid. ¹H NMR (400 MHz, Acetone): δ 13.10 (s br, 1H); 8.94 (s, 1H); 8.77 (s, 1H); 8.70-8.60 (m br, 2H); 8.39 (d, 2H); 8.03 (t, 1H); 7.75 (dd, 1H); 7.69 (dd, 1H); 4.92 (s, 1H); 3.05 (m, 2H); 1.95 (m, 2H); 1.33 (s, 6H).

### Example 123

### (+)-2-[9-chloro-6-(3,4-dihydroxy-3-methylbut-1-yn-1-yl)-1H-phenanthro[9,10-d]imidazol-2-yl]isophthalonitrile

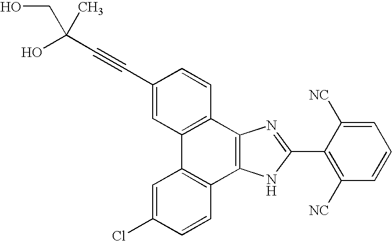

### Step 1: 2-[6-chloro-9-(3-methylbut-3-en-1-yn-1-yl)-1H-phenanthro[9,10-d]imidazol-2-yl]isophthalonitrile

To a stirred suspension of 2-[9-chloro-6-(3-hydroxy-3-methylbut-1-yn-1-yl)-1H-phenanthro[9,10-d]imidazol-2-yl]isophthalonitrile from Example 40 (120 mg, 0.26 mmol) in benzene (4 mL) was added Burgess Reagent (70 mg, 0.29 mmol) and refluxed for 2 hours under N₂. The resulting reaction mixture was diluted with EtOAc (20 mL). This EtOAc solution was washed with water, brine and dried over MgSO₄. After removing the drying agent via filtration, the organic solution was concentrated under reduced pressure. The residue was purified by silica gel column chromatography (eluted with 50/50 EtOAc/hexane) to yield 90 mg of 2-[6-chloro-9-(3-methylbut-3-en-1-yn-1-yl)-1H-phenanthro[9,10-d]imidazol-2-yl]isophthalonitrile as a yellow solid.

### Step 2: (+)-2-[9-chloro-6-(3,4-dihydroxy-3-methylbut-1-yn-1-yl)-1H-phenanthro[9,10-d]imidazol-2-yl]isophthalonitrile

To a stirred suspension of 2-[6-chloro-9-(3-methylbut-3-en-1-yn-1-yl)-1H-phenanthro[9,10-d]imidazol-2-yl]isophthalonitrile from Step 1 (22 mg, 0.05 mmol) in 50/50 t-BuOH/H₂O (0.5 mL) was added AD-mix-α (70 mg) at 0° C. The mixture was left stirring at 0° C. for 24 hours. The resulting reaction mixture was treated with saturated Na₂S₂O₃ aqueous solution and stirred for 10 minutes, diluted with water and extracted with EtOAc. This EtOAc solution was washed with water, brine and dried over MgSO₄. After removing the drying agent via filtration, the organic solution was concentrated under reduced pressure. The residue was purified by silica gel column chromatography (eluted with 50/50 EtOAc/hexane to 95/5 EtOAc/MeOH) to yield 19 mg of yellow solid. This same procedure was repeated with AD-mix-α to yield another 19 mg of yellow solid. These two yellow solids were combined to give the racemic 2-[9-chloro-6-(3,4-dihydroxy-3-methylbut-1-yn-1-yl)-1H-phenanthro[9,10-d]imidazol-2-yl]isophthalonitrile.

¹H NMR (400 MHz, Acetone): δ 8.84 (d, 1H); 8.80 (s, 1H); 8.57 (d, 1H); 8.47 (d, 1H); 8.39 (d, 2H); 8.03 (t, 1H); 7.77 (dd, 8.6 Hz, 1H); 7.71 (dd, 1H); 4.56 (s, 1H); 4.30 (s, 1H); 3.67 (q, 2H); 1.56 (s, 3H).

### Example 135

### 2-[9-chloro-6-(2-hydroxy-2-methylpropyl)-1H-phenanthro[9,10-d]imidazol-2-yl]isophthalonitrile

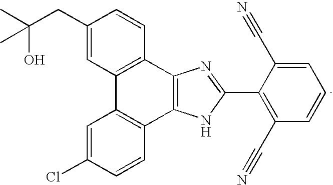

### Step 1: 2-(6-bromo-9-chloro-1-{[2-(trimethylsilyl)ethoxy]methyl}-1H-phenanthro[9,10-d]imidazol-2-yl)isophthalonitrile

To a solution of 2-(6-bromo-9-chloro-1H-phenanthro[9,10-d]imidazol-2-yl)isophthalonitrile (5 g, 10.9 mmol) from Example 36 in THF (30 mL) was added NaH (60% dispersion in oil, 1.31 g, 32.7 mmol). The mixture was stirred at room temperature for 10 minutes, after which 2-(trimethylsilyl)ethoxymethylchloride (5.8 mL, 32.7 mmol) was added. After 1 hour, the reaction was quenched by slow addition of water. The aqueous layer was extracted with ethyl acetate, the organic layer washed once with water, once with brine, dried over anhydrous MgSO₄ and concentrated to afford crude 2-(6-bromo-9-chloro-1-{[2-(trimethylsilyl)ethoxy]methyl}-1H-phenanthro[9,10-d]imidazol-2-yl)isophthalonitrile (6.06 g).

### Step 2: 2-(9-chloro-6-(2-oxopropyl)-1-{[2-(trimethylsilyl)ethoxy]methyl)-1H-phenanthro[9,10-d]imidazol-2-yl)isophthalonitrile

A solution of tributyl(methoxy)stannane (4.5 mL, 15.5 mmol), isopropenylacetate (1.7 mL, 15.5 mmol), 2-(6-bromo-9-chloro-1-{[2-(trimethylsilyl)ethoxy]methyl}-1H-phenanthro[9,10-d]imidazol-2-yl)isophthalonitrile from Step 1 above (6.06 g, 10.3 mmol), palladium (II) acetate (0.232 g, 1.03 mmol) and tri-o-tolylphosphine (0.628 g, 2.07 mmol) in toluene (50 mL) was heated at 100° C. overnight. The reaction mixture was quenched with water and ethyl acetate. Following usual workup and chromatography on silica (50% ethyl acetate in hexanes), 2-(9-chloro-6-(2-oxopropyl)-1-{[2-(trimethylsilyl)ethoxy]methyl)-1H-phenanthro[9,10-d]imidazol-2-yl)isophthalonitrile (2.8 g) was isolated as a yellow-orange solid.

### Step 3: 2-(9-chloro-6-(2-hydroxy-2-methylpropyl)-1-{[2-(trimethylsilyl)ethoxy]methyl}-1H-phenanthro[9,10-d]imidazol-2-yl)isophthalonitrile

To a round bottomed flask at −78° C. charged with TiCl₄ (1 M in CH₂Cl₂, 20 mL), was added methyllithium (1.6 M in diethyl ether, 12.5 mL). The resulting deep red solution was stirred at −78° C. for 15 minutes and then added via cannula to a 0° C. solution of 2-(9-chloro-6-(2-oxopropyl)-1-{[2-(trimethylsilyl)ethoxy]methyl)-1H-phenanthro[9,10-d]imidazol-2-yl)isophthalonitrile (2.8 g, 5.0 mmol) from Step 2 above, in diethyl ether (10 mL). The resulting mixture was stirred at 0° C. for 3 h, then quenched with saturated ammonium chloride. The aqueous layer was extracted with ethyl acetate. The organic layer was washed with brine, dried over MgSO₄, filtered and concentrated. The crude material was purified by flash chromatography on silica (50% ethyl acetate in hexanes) to provide 2-(9-chloro-6-(2-hydroxy-2-methylpropyl)-1-{[2-(trimethylsilyl)ethoxy]methyl}-1H-phenanthro[9,10-d]imidazol-2-yl)isophthalonitrile (1.94 g),

### Step 4: 2-[9-chloro-6-(2-hydroxy-2-methylpropyl)-1H-phenanthro[9,10-d]imidazol-2-yl]isophthalonitrile

2-(9-chloro-6-(2-hydroxy-2-methylpropyl)-1-{[2-(trimethylsilyl)ethoxy]methyl}-1H-phenanthro[9,10-d]imidazol-2-yl)isophthalonitrile (1.94 g) from Step 3 above was dissolved in TBAF (1 M in THF, 20 mL). The mixture was heated at reflux for 5 h and then quenched with water. The aqueous layer was extracted with ethyl acetate. The organic layer was washed with brine, dried over MgSO₄, filtered and concentrated. The crude material was purified by flash chromatography on silica (50% ethyl acetate in hexanes) to provide 2-[9-chloro-6-(2-hydroxy-2-methylpropyl)-1H-phenanthro[9,10-d]imidazol-2-yl]isophthalonitrile (500 mg) as a yellow solid.

¹H NMR δ (ppm)(400 MHz, Acetone-d₆): 13.13 (1H, bs), 8.87 (1H, s), 8.77 (1H, s), 8.58 (1H, m), 8.43 (1H, m), 8.35 (2H, d, J=7.9 Hz), 7.99 (1H, t, J=7.9 Hz), 7.73 (2H, dd, J=1.9, 8.6 Hz), 3.51 (1H, bs), 3.08 (2H, s), 1.26 (6H, s).

### Example 160

### 2-[9-(cyclopropylmethoxy)-6-(3-hydroxy-3-methylbut-1-yn-1-yl)-1H-phenanthro[9,10-d]imidazol-2-yl]isophthalonitrile

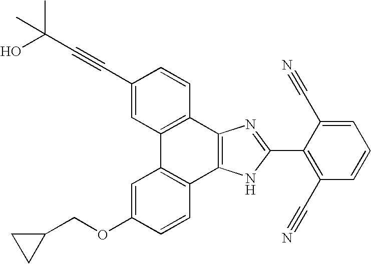

### Step 1: 1-bromo-4-[2-(4-methoxyphenyl)vinyl]benzene

This stillbene was prepared as described in Step 1 of Example 36, substituting p-anisaldehyde for 4-chlorobenzaldehyde.

### Step 2: 3-bromo-6-methoxyphenanthrene

This phenanthrene was prepared as described in Step 2 of Example 36, substituting 1-bromo-4-[2-(4-methoxyphenyl)vinyl]benzene from Step 1 above for 1-bromo-4-[2-(4-chlorophenyl)vinyl]benzene and performing the irradiation for 4 days.

### Step 3: 3-bromo-6-methoxyphenanthrene-9,10-dione

This quinone was prepared as described in Step 3, Example 36, substituting 3-bromo-6-methoxyphenanthrene from Step 2 above for 3-bromo-6-chlorophenanthrene.

### Step 4: 3-bromo-6-hydroxyphenanthrene-9,10-dione

A mixture of 3-bromo-6-methoxyphenanthrene-9,10-dione from Step 3 above and excess BBr₃ in CH₂Cl₂ was stirred at room temperature to afford 3-bromo-6-hydroxyphenanthrene-9,10-dione which was used directly in the next step (Step 5 below).

### Step 5: 3-bromo-6-(cyclopropylmethoxy)phenanthrene-9,10-dione

A solution of 3-bromo-6-hydroxyphenanthrene-9,10-dione from Step 4 in acetone was treated with excess potassium carbonate, potassium iodide and (bromomethyl)cyclopropane. The mixture was heated at reflux overnight, followed by standard workup to yield 3-bromo-6-(cyclopropylmethoxy)phenanthrene-9,10-dione.

### Step 6: 6-bromo-9-(cyclopropylmethoxy)-2-(2,6-dibromophenyl)-1H-phenanthro[9,10-d]imidazole

This imidazole was prepared as described in Step 4 of Example 36, substituting 3-bromo-6-(cyclopropylmethoxy)phenanthrene-9,10-dione from Step 5 above for 3-bromo-6-chlorophenanthrene-9,10-dione

### Step 7: 2-[6-bromo-9-(cyclopropylmethoxy)-1H-phenanthro[9,10-d]imidazol-2-yl]isophthalonitrile

This imidazole was prepared as described in Step 5 of Example 36, substituting 6-bromo-9-(cyclopropylmethoxy)-2-(2,6-dibromophenyl)-1H-phenanthro[9,10-d]imidazole from Step 6 above for 9-bromo-6-chloro-2-(2,6-dibromophenyl)-1H-phenanthro[9,10-d]imidazole. The impurity present in the product was removed by Sharpless dihydroxylation.

### Step 8: 2-[9-(cyclopropylmethoxy)-6-(3-hydroxy-3-methylbut-1-yn-1-yl)-1H-phenanthro[9,10-d]imidazol-2-yl]isophthalonitrile

This imidazole was prepared as described in Step 6, Example 40, substituting 2-[6-bromo-9-(cyclopropylmethoxy)-1H-phenanthro[9,10-d]imidazol-2-yl]isophthalonitrile from Step 7 above for 2-(9-bromo-6-chloro-1H-phenanthro[9,10-d]imidazol-2-yl)isophthalonitrile. ¹H NMR δ (ppm)(400 MHz, Acetone-d₆): 13.04 (1H, bs), 8.88 (1H, d, J=5.7 Hz), 8.49 (2H, m), 8.33 (3H, m), 7.99 (1H, t, J=8.0 Hz), 7.73 (1H, d, J=8.2 Hz), 7.43 (1H, d, J=8.8 Hz), 4.54 (1H, bs), 4.17 (2H, d, J=6.8 Hz), 1.63 (6H, s), 1.48-1.36 (1H, m), 0.68 (1H, m), 0.49-0.45 (1H, m).

### Example 168

### 2-[9-(cyclopropylmethoxy)-6-(2-hydroxy-2-methylpropyl)-1H-phenanthro[9,10-d]imidazol-2-yl]isophthalonitrile

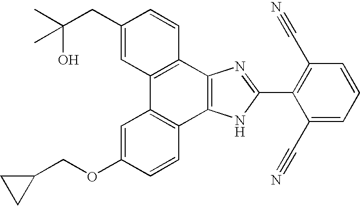

This compound was prepared by two routes as described below:

### Route A:

### Step 1: 6-bromophenanthren-3-ol

To a flask containing BBr₃ (1 M in CH₂Cl₂, 17 mL) at 0° C. was added a solution of 3-bromo-6-methoxyphenanthrene (1 g, 3.5 mmol) from Step 2, Example 160 in CH₂Cl₂ (10 mL). The reaction mixture was warmed to room temperature and stirred for 30 minutes, after which it was quenched with water. The aqueous layer was extracted with CH₂Cl₂. The organic layer was dried over MgSO₄, filtered and concentrated to yield crude 6-bromophenanthren-3-ol.

### Step 2: 3-bromo-6-(cyclopropylmethoxy)phenanthrene

A mixture of 6-bromophenanthren-3-ol (0.823 g, 3.02 mmol) from Step 1 above, (bromomethyl)cyclopropane (0.5 mL, 5.4 mmol), potassium carbonate (2.5 g, 18 mmol) and potassium iodide (5 mg) in acetone (50 mL) was heated at reflux for 3 days. Water was then added and the reaction mixture extracted with ethyl acetate The organic layer was washed with brine, dried over MgSO₄, filtered and concentrated. The crude material was purified by flash chromatography on silica (100% hexanes) to provide 3-bromo-6-(cyclopropylmethoxy)phenanthrene (0.859 g, 87%).

### Step 3: 1-[6-(cyclopropylmethoxy)-3-phenanthryl]acetone

This phenanthrene was prepared as described in Step 2 of Example 135, substituting 3-bromo-6-(cyclopropylmethoxy)phenanthrene from Step 2 above for 2-(6-bromo-9-chloro-1-([2-{trimethylsilyl)ethoxy]methyl}-1H-phenanthro[9,10-d]imidazol-2-yl)isophthalonitrile.

### Step 4: 1-[6-(cyclopropylmethoxy)-3-phenanthryl]-2-methylpropan-2-ol

This phenanthrene was prepared as described in Step 3 of Example 135, substituting 1-[6-(cyclopropylmethoxy)-3-phenanthryl]acetone from Step 3 above for 2-(9-chloro-6-(2-oxopropyl)-1-{[2-(trimethylsilyl)ethoxy]methyl)-1H-phenanthro[9,10-d]imidazol-2-yl)isophthalonitrile. The crude product was used directly in the next reaction.

### Step 5: tert-butyl(2-[6-(cyclopropylmethoxy)-3-phenanthryl]-1,1-dimethylethoxy)dimethylsilane

To a solution of crude 1-[6-(cyclopropylmethoxy)-3-phenanthryl]-2-methylpropan-2-ol from Step 4 above in THF (10 mL), was added sodium hydride (60% dispersion in oil, 0.27 g, 6.79 mmol). The mixture was heated at reflux for 2 minutes, then cooled to room temperature. Tert-butyldimethylsilylchloride (0.512 g, 3.39 mmol) was added and the reaction mixture heated at reflux for 2 h. After usual workup of the reaction, tert-butyl(2-[6-(cyclopropylmethoxy)-3-phenanthryl]-1,1-dimethylethoxy)dimethylsilane (0.5 g) was obtained, which was used as crude material for the next step.

### Step 6: 3-(2-{[tert-butyl(dimethyl)silyl]oxy}-2-methylpropyl)-6-(cyclopropylmethoxy)phenanthrene-9,10-dione

To a solution of tert-butyl(2-[6-(cyclopropylmethoxy)-3-phenanthryl]-1,1-dimethylethoxy)dimethylsilane (0.5 g, 1.15 mmol) from Step 5 above, in acetic acid (10 mL), was added CrO₃ (0.346 g, 3.46 mmol). The mixture was stirred at 50° C. for 30 min, cooled down to room temperature, poured into water and stirred for 15 minutes. The suspension was filtered, washed with water and pumped under reduced pressure to afford 3-(2-{[tert-butyl(dimethyl)silyl]oxy}-2-methylpropyl)-6-(cyclopropylmethoxy)phenanthrene-9,10-dione.

### Step 7: 6-(2-{[tert-butyl(dimethyl)silyl]oxy}-2-methylpropyl)-9-(cyclopropylmethoxy)-2-(2,6-dibromophenyl)-1H-phenanthro[9,10-d]imidazole

To a solution of 3-(2-{[tert-butyl(dimethyl)silyl]oxy}-2-methylpropyl)-6-(cyclopropylmethoxy)phenanthrene-9,10-dione (1.15 mmol) from Step 6 above in acetic acid (10 ml), was added ammonium acetate (1.78 g, 23 mmol) and dibromobenzaldehyde (0.42 g, 1.5 mmol). The mixture was stirred at 70° C. for 1 h, cooled down to room temperature, poured into water and stirred for 5 minutes. The resulting solid was washed with water and diethyl ether. The crude material was purified by flash chromatography on silica (30% ethyl acetate in hexanes) to afford 6-(2-{[tert-butyl(dimethyl)silyl]oxy}-2-methylpropyl)-9-(cyclopropylmethoxy)-2-(2,6-dibromophenyl)-1H-phenanthro[9,10-d]imidazole (0.223 g) as a yellow solid.

### Step 8: 1-[9-(cyclopropylmethoxy)-2-(2,6-dibromophenyl)-1H-phenanthro[9,10-d]imidazol-6-yl]-2-methylpropan-2-ol

TBAF (1 M in THF, 10 mL) was added to a flask containing 6-(2-{[tert-butyl(dimethyl)silyl]oxy}-2-methylpropyl)-9-(cyclopropylmethoxy)-2-(2,6-dibromophenyl)-1H-phenanthro[9,10-d]imidazole (0.223 g, 0.31 mmol) from Step 7 above, at room temperature. The resulting solution was heated at reflux for 36 h, after which water was added to the reaction mixture. The aqueous layer was extracted with ethyl acetate, the organic layer dried over MgSO₄, filtered and concentrated. The crude product was used directly in the next reaction (Step 9 below).

### Step 9: 2-[9-(cyclopropylmethoxy)-6-(2-hydroxy-2-methylpropyl)-1H-phenanthro[9,10-d]imidazol-2-yl]isophthalonitrile

This imidazole was prepared as described in Step 5 of Example 36, substituting crude 1-[9-(cyclopropylmethoxy)-2-(2,6-dibromophenyl)-1H-phenanthro[9,10-d]imidazol-6-yl]-2-methylpropan-2-ol from Step 8 above for 9-bromo-6-chloro-2-(2,6-dibromophenyl)-1H-phenanthro[9,10-d]imidazole. ¹H NMR δ (ppm)(400 MHz, Acetone-d₆): 12.96 (1H, bs), 8.70 (1H, m), 8.59 (1H, m), 8.32 (3H, d, J=8.0 Hz), 8.28 (1H, m), 7.95 (1H, t, J=7.9 Hz), 7.67 (1H, d, J=8.1 Hz), 7.38 (1H, d, J=8.7 Hz), 4.09 (2H, d, J=6.9 Hz), 3.46 (1H, bs), 3.05 (2H, s), 1.38-1.34 (1H, m), 1.25 (6H, s), 0.67-0.63 (2H, m), 0.45-0.41 (2H, m).

### Route B:

### Step 1: 3-bromo-6-(cyclopropylmethoxy)phenanthrene-9,10-dione

This quinone was prepared either as described in Step 5, Example 160, or by following the procedure described in Step 3, Example 36, substituting 3-bromo-6-(cyclopropylmethoxy)phenanthrene from Step 2 of Route A above for 3-bromo-6-chlorophenanthrene.

### Step 2: 6-bromo-9-(cyclopropylmethoxy)-2-(2,6-dibromophenyl)-1H-phenanthro[9,10-d]imidazole

This imidazole was prepared as described in Step 6 of Example 160.

### Step 3: 2-[6-bromo-9-(cyclopropylmethoxy)-1H-phenanthro[9,10-d]imidazol-2-yl]isophthalonitrile

This imidazole was prepared as described in Step 7 of Example 160.

### Step 4: 2-(6-bromo-9-(cyclopropylmethoxy)-1-{[2-(trimethylsilyl)ethoxy]methyl}-1H-phenanthro[9,10-d]imidazol-2-yl)isophthalonitrile

This SEM-protected imidazole was prepared as described in Step 2, Example 87, substituting 2-[6-bromo-9-(cyclopropylmethoxy)-1H-phenanthro[9,10-d]imidazol-2-yl]isophthalonitrile from Step 3 above for 6,9-dibromo-2-(2,6-dibromophenyl)-1H-phenanthro[9,10-d]imidazole.

### Step 5: 2-(9-(cyclopropylmethoxy)-6-(2-oxopropyl)-1-{[2-(trimethylsilyl)ethoxy]methyl}-1H-phenanthro[9,10-d]imidazol-2-yl)isophthalonitrile

This imidazole was prepared as described in Step 2, Example 135, substituting 2-(6-bromo-9-(cyclopropylmethoxy)-1-{[2-(trimethylsilyl)ethoxy]methyl}-1H-phenanthro[9,10-d]imidazol-2-yl)isophthalonitrile from Step 4 above for 2-(6-bromo-9-chloro-1-([2-(trimethylsilyl)ethoxy]methyl)-1H-phenanthro[9,10-d]imidazol-2-yl)isophthalonitrile.

### Step 6: 2-(9-(cyclopropylmethoxy)-6-(2-hydroxy-2-methylpropyl)-1-{[2-(trimethylsilyl)ethoxy]methyl}-1H-phenanthro[9,10-d]imidazol-2-yl)isophthalonitrile

This imidazole was prepared as described in Step 3, Example 135, substituting 2-(9-(cyclopropylmethoxy)-6-(2-oxopropyl)-1-{[2-(trimethylsilyl)ethoxy]methyl}-1H-phenanthro[9,10-d]imidazol-2-yl)isophthalonitrile from Step 5 above for of 2-(9-chloro-6-(2-oxopropyl)-1-{[2-(trimethylsilyl)ethoxy]methyl)-1H-phenanthro[9,10-d]imidazol-2-yl)isophthalonitrile.

### Step 7: 2-[9-(cyclopropylmethoxy)-6-(2-hydroxy-2-methylpropyl)-1H-phenanthro[9,10-d]imidazol-2-yl]isophthalonitrile

Crude 2-(9-(cyclopropylmethoxy)-6-(2-hydroxy-2-methylpropyl)-1-{[2-(trimethylsilyl)ethoxy]methyl}-1H-phenanthro[9,10-d]imidazol-2-yl)isophthalonitrile (1.37 mmol) from Step 6 above was dissolved in TBAF (1 M in THF, 10 mL) and the mixture heated at reflux for 1.5 h. Water was added, and the aqueous layer extracted with ethyl acetate. The organic layer was dried over MgSO₄, filtered and concentrated. The material was purified by flash chromatography on silica (70% ethyl acetate in hexanes) to afford 2-[9-(cyclopropylmethoxy)-6-(2-hydroxy-2-methylpropyl)-1H-phenanthro[9,10-d]imidazol-2-yl]isophthalonitrile (240 mg).

### Example 172

### 2-[9-(2-cyclopropylethoxy)-6-(2-hydroxy-2-methylpropyl)-1H-phenanthro[9,10-d]imidazol-2-yl]-5-fluoroisophthalonitrile

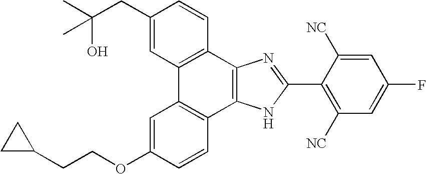

### Step 1: 3-bromo-6-(2-cyclopropylethoxy)phenanthrene

To a mixture of 6-bromophenanthren-3-ol (3 g, 11 mmol) from Step 1 of Route A of Example 168, 2-cyclopropylethanol (2.85 g, 33 mmol) and triphenylphosphine (5.78 g, 22 mmol) in THF (50 mL) was added di-tert-butylazodicarboxylate (5.08 g, 22 mmol). The reaction mixture was stirred at room temperature overnight, then quenched with water. The aqueous layer was extracted with ethyl acetate. The combined organic layer was washed with brine, dried over MgSO₄, filtered and concentrated. The material was purified by flash chromatography on silica (100% hexanes) to afford 3-bromo-6-(2-cyclopropylethoxy)phenanthrene.

### Step 2: 1-[6-(2-cyclopropylethoxy)-3-phenanthryl]-2-methylpropan-2-ol

This phenanthrene could either be prepared via the two-step process described in Steps 3 and 4 of Route A of Example 168, substituting 3-bromo-6-(2-cyclopropylethoxy)phenanthrene from Step 1 above for 3-bromo-6-(cyclopropylmethoxy)phenanthrene, or by following the procedure below:

To a solution of 3-bromo-6-(2-cyclopropylethoxy)phenanthrene (11 mmol) from Step 1 above in THF (75 mL) at ˜78° C. was successively added methyllithium (1.6 M in diethyl ether, 1 mL) and butyllithium (2.5 M in hexanes, 5.3 mL). The mixture was stirred at −78° C. for 30 minutes, after which isobutylene oxide (2.9 mL, 33 mmol) was added, followed by BF₃.OEt₂ (4.2 mL, 33 mmol). The reaction mixture was stirred at −78° C. for 1 h, then quenched with 1 M HCl. The aqueous layer was extracted with ethyl acetate. The combined organic layer was washed with brine, dried over MgSO₄, filtered and concentrated. The material was purified by flash chromatography on silica (10% ethyl acetate in hexanes) to afford 1-[6-(2-cyclopropylethoxy)-3-phenanthryl]-2-methylpropan-2-ol (1.33 g) as a yellow oil.

### Step 3: tert-butyl(2-[6-(2-cyclopropylethoxy)-3-phenanthryl]-1,1-dimethylethoxy)dimethylsilane

This phenanthrene was prepared as described in Step 5 of Route A of Example 168, substituting 1-[6-(2-cyclopropylethoxy)-3-phenanthryl]-2-methylpropan-2-ol from Step 2 above for 1-[6-(cyclopropylmethoxy)-3-phenanthryl]-2-methylpropan-2-ol.

### Step 4: 3-(2-{[tert-butyl(dimethyl)silyl]oxy}-2-methylpropyl)-6-(2-cyclopropylethoxy)phenanthrene-9,10-dione

This quinone was prepared as described in Step 6 of Route A of Example 168, substituting tert-butyl(2-[6-(2-cyclopropylethoxy)-3-phenanthryl]-1,1-dimethylethoxy)dimethylsilane from Step 3 above for tert-butyl(2-[6-(cyclopropylmethoxy)-3-phenanthryl]-1,1′-dimethylethoxy)dimethylsilane.

### Step 5: 6-(2-{[tert-butyl(dimethyl)silyl]oxy})-2-methylpropyl)-9-(2-cyclopropylethoxy)-2-(2,6-dibromo-4-fluorophenyl)-1H-phenanthro[9,10-d]imidazole

This imidazole was prepared as described in Step 7 of Route A of Example 168, substituting 3-(2-{[tert-butyl(dimethyl)silyl]oxy}-2-methylpropyl)-6-(2-cyclopropylethoxy)phenanthrene-9,10-dione from Step 4 above for 3-(2-{([tert-butyl(dimethyl)silyl]oxy}-2-methylpropyl)-6-(cyclopropylmethoxy)phenanthrene-9,10-dione and 2,6-dibromo-4-fluorobenzaldehyde for dibromobenzaldehyde.

### Step 6: 1-[9-(2-cyclopropylethoxy)-2-(2,6-dibromo-4-fluorophenyl)-1H-phenanthro[9,10-d]imidazol-6-yl]-2-methylpropan-2-ol

This imidazole was prepared as described in Step 8 of Route A of Example 168, substituting 6-(2-{[tert-butyl(dimethyl)silyl]oxy}-2-methylpropyl)-9-(2-cyclopropylethoxy)-2-(2,6-dibromo-4-fluorophenyl)-1H-phenanthro[9,10-d]imidazole from Step 5 above for 6-(2-{[tert-butyl(dimethyl)silyl]oxy}-2-methylpropyl)-9-(cyclopropylmethoxy)-2-(2,6-dibromophenyl)-1H-phenanthro[9,10-d]imidazole.

### Step 7: 2-[9-(2-cyclopropylethoxy)-6-(2-hydroxy-2-methylpropyl)-1H-phenanthro[9,10-d]imidazol-2-yl]-5-fluoroisophthalonitrile

This imidazole was prepared as described in Step 5 of Example 36, substituting 1-[9-(2-cyclopropylethoxy)-2-(2,6-dibromo-4-fluorophenyl)-1H-phenanthro[9,10-d]imidazol-6-yl]-2-methylpropan-2-ol from Step 6 above for 9-bromo-6-chloro-2-(2,6-dibromophenyl)-1H-phenanthro[9,10-d]imidazole. ¹H NMR δ (ppm)(400 MHz, Acetone-d₆): 12.95 (1H, bs), 8.70 (1H, m), 8.58 (1H, m), 8.28 (4H, m), 7.67 (1H, d, J=8.1 Hz), 7.40 (1H, d, J=9.1 Hz), 4.31 (2H, t, J=6.5 Hz), 3.43 (1H, bs), 3.05 (2H, s), 1.78 (2H, q, J=6.7 Hz), 1.26 (6H, s), 0.98 (1H, m), 0.54-0.48 (2H, m), 0.20-0.18 (2H, m).

### Example 180

### 2-[6-(2-hydroxy-2-methylpropyl)-9-(4,4,4-trifluorobutoxy)-1H-phenanthro[9,10-d]imidazol-2-yl]isophthalonitrile

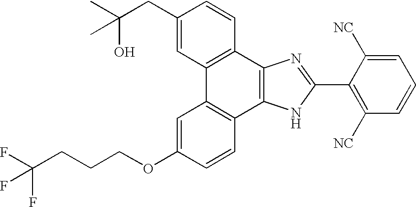

### Step 1: 3-bromo-6-(4,4,4-trifluorobutoxy)phenanthrene

This phenanthrene was prepared as described in Step 2 of Route A of Example 168, substituting 4,4,4-trifluoro-1-iodobutane for (bromomethyl)cyclopropane.

### Step 2: 2-methyl-1-[6-(4,4,4-trifluorobutoxy)-3-phenanthryl]propan-2-ol

This phenanthrene was prepared as described in Step 2, Example 172, substituting 3-bromo-6-(4,4,4-trifluorobutoxy)phenanthrene from Step 1 above for 3-bromo-6-(2-cyclopropylethoxy)phenanthrene.

### Step 3: tert-butyl(1,1-dimethyl-2-[6-(4,4,4-trifluorobutoxy)-3-phenanthryl]ethoxy)dimethylsilane

This phenanthrene was prepared as described in Step 5 of Route A of Example 168, substituting 2-methyl-1-[6-(4,4,4-trifluorobutoxy)-3-phenanthryl]propan-2-ol from Step 2 above for 1-[6-(cyclopropylmethoxy)-3-phenanthryl]-2-methylpropan-2-ol.

### Step 4: 3-(2-{[tert-butyl(dimethyl)silyl]oxy}-2-methylpropyl)-6-(4,4,4-trifluorobutoxy)phenanthrene-9,10-dione

This quinone was prepared as described in Step 6 of Route A of Example 168, substituting tert-butyl(1,1-dimethyl-2-[6-(4,4,4-trifluorobutoxy)-3-phenanthryl]ethoxy)dimethylsilane from Step 3 above for tert-butyl(2-[6-(cyclopropylmethoxy)-3-phenanthryl]-1,1-dimethylethoxy)dimethylsilane.

### Step 5: 6-(2-([tert-butyl(dimethyl)silyl]oxy)-2-methylpropyl)-2-(2,6-dibromophenyl)-9-(4,4,4-trifluorobutoxy)-1H-phenanthro[9,10-d]imidazole

This imidazole was prepared as described in Step 7 of Route A of Example 168, substituting 3-(2-{[tert-butyl(dimethyl)silyl]oxy}-2-methylpropyl)-6-(4,4,4-trifluorobutoxy)phenanthrene-9,10-dione from Step 4 above for 3-(2-{[tert-butyl(dimethyl)silyl]oxy}-2-methylpropyl)-6-(cyclopropylmethoxy)phenanthrene-9,10-dione.

### Step 6: 1-[2-(2,6-dibromophenyl)-9-(4,4,4-trifluorobutoxy)-1H-phenanthro[9,10-d]imidazol-6-yl]-2-methylpropan-2-ol

This imidazole was prepared as described in Step 8 of Route A of Example 168, substituting 6-(2-([tert-butyl(dimethyl)silyl]oxy)-2-methylpropyl)-2-(2,6-dibromophenyl)-9-(4,4,4-trifluorobutoxy)-1H-phenanthro[9,10-d]imidazole from Step 5 above for 6-(2-{[tert-butyl(dimethyl)silyl]oxy}-2-methylpropyl)-9-(cyclopropylmethoxy)-2-(2,6-dibromophenyl)-1H-phenanthro[9,10-d]imidazole.

### Step 7: 2-[6-(2-hydroxy-2-methylpropyl)-9-(4,4,4-trifluorobutoxy)-1H-phenanthro[9,10-d]imidazol-2-yl]isophthalonitrile

This imidazole was prepared as described in Step 5 of Example 36, substituting 1-[2-(2,6-dibromophenyl)-9-(4,4,4-trifluorobutoxy)-1H-phenanthro[9,10-d]imidazol-6-yl]-2-methylpropan-2-ol from Step 6 above for 9-bromo-6-chloro-2-(2,6-dibromophenyl)-1H-phenanthro[9,10-d]imidazole. ¹H NMR δ (ppm)(400 MHz, Acetone-d₆): 12.95 (1H, bs), 8.72 (2H, m), 8.33 (4H, m), 7.96 (1H, t, J=7.9 Hz), 7.68 (1H, d, J=8.1 Hz), 7.42 (1H, d, J=9.5 Hz), 4.36 (2H, t, J=6.0 Hz), 3.45 (1H, bs), 3.05 (2H, s), 2.57-2.51 (2H, m), 2.20-2.12 (2H, m), 1.25 (6H, s).

### Assays for Determining Biological Activity

### Inhibition of Prostaglandin E Synthase Activity

Compounds are tested as inhibitors of prostaglandin E synthase activity in microsomal prostaglandin E synthases, whole cell and in vivo assays. These assays measure prostaglandin E2 (PGE2) synthesis using either Enzymatic Immunoassay (EIA) or mass spectrometry. Cells used for microsomal preparation are CHO-K1 cells transiently transfected with plasmids encoding the human mPGES-1 cDNA. Cells used for cell-based experiments are human A549 (which express human mPGES-1). Guinea pigs are used to test the activity of selected compounds in vivo. In all these assays, 100% activity is defined as the PGE2 production in vehicle-treated samples. IC₅₀ and ED₅₀ represent the concentration or dose of inhibitor required to inhibit PGE2 synthesis by 50% as compared to the uninhibited control.

### Microsomal Prostaglandin E Synthase Assay

Prostaglandin E synthase microsomal fractions are prepared from CHO-K1 cells transiently transfected with plasmid encoding the human mPGES-1 cDNA. Microsomes are then prepared and the PGES assay begins with the incubation of 5 μg/ml microsomal PGES-1 with compound or DMSO (final 1%) for 20-30 minutes at room temperature. The enzyme reactions are performed in 200 mM KPi pH 7.0, 2 mM EDTA and 2.5 mM GSH-reduced form. The enzymatic reaction is then initiated by the addition of 1 μM final PGH2 substrate prepared in isopropanol (3.5% final in assay well) and incubated at room temperature for 30 seconds. The reaction is terminated by the addition of SnCl₂ in 1N HCl (1 mg/ml final). Measurement of PGE_{2′} production in the enzyme reaction aliquots is done by EIA using a standard commercially available kit (Cat #: 901-001 from Assay Designs).

Data from this assay for representative compounds is shown in the table below. The potency is expressed as IC₅₀ and the value indicated is an average of at least n=3.

| Ex. | h-CHO (nM) |
|---|---|
| 1 | 1.9 |
| 5 | 2.1 |
| 8 | 2 |
| 9 | 1.9 |
| 14 | 1.8 |
| 20 | 13.1 |
| 21 | 12 |
| 25 | 1.3 |
| 23 | 2.1 |
| 36 | 1.2 |
| 37 | 9.9 |
| 40 | 0.9 |
| 45 | 2534 |
| 46 | 1.5 |
| 48 | 0.9 |
| 51 | 4.8 |
| 55 | 1.1 |
| 56 | 1.7 |
| 65 | 1.5 |
| 68 | 1.5 |
| 73 | 1.7 |
| 76 | 3.7 |
| 87 | 1.9 |
| 88 | 1.3 |
| 91 | 1 |
| 93 | 1.2 |
| 95 | 2.4 |
| 98 | 0.9 |
| 99 | 1.2 |
| 117 | 0.7 |

### Human A549 Whole Cell Prostaglandin E Synthase Assay

### Rationale

Whole cells provide an intact cellular environment for the study of cellular permeability and biochemical specificity of anti-inflammatory compounds such as prostaglandin E synthase inhibitors. To study the inhibitory activities of these compounds, human A549 cells are stimulated with 10 ng/ml recombinant human IL-1 for 24 hours. The production of PGE2 and PGF_{2α} are measured by EIA at the end of the incubation as readouts for selectivity and effectiveness against mPGES-1-dependent PGE2 production.

### Methods

Human A549 cells specifically express human microsomal prostaglandin E synthase-1 and induce its expression following treatment with IL-1β for 24 hours. 2.5×10⁴ cells seeded in 100 ul/well (96-well plate) and incubated overnight under standard conditions. 100 ul of cell culture media containing 10 ng/ml IL-11 is then added to the cells followed by the addition of either 2% FBS containing RPMI or 50% FBS containing RPMI. 2 μl of drugs or vehicle (DMSO) are then added and samples are mixed immediately. Cells are incubated for 24 hours and following the incubation 175 μl of medium is harvested and assayed for PGE₂ and PGF_{2α} contents by EIA.

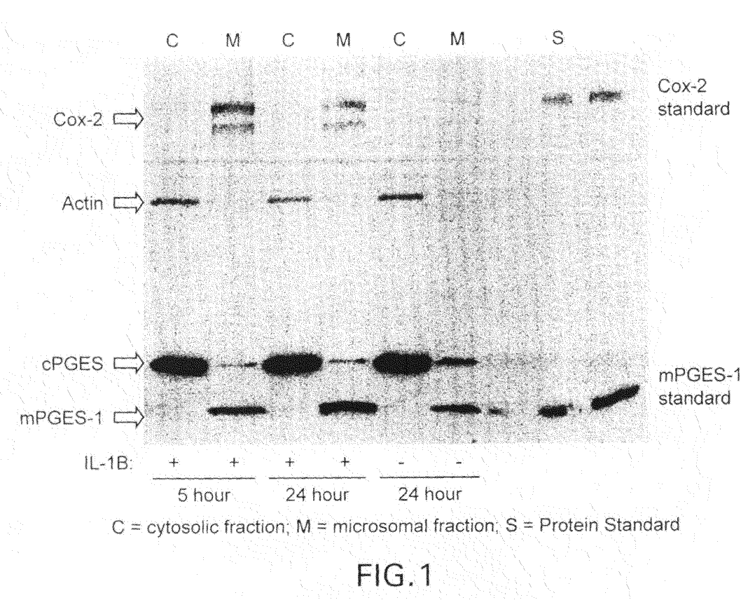

A549 cells (human lung adenocarcinoma cell line) were treated with Example 81 following the above procedure. The expression of mPGES-1 is induced by the cytokine IL-1β. Example 81, a selective mPGES-1 inhibitor, inhibits IL-1β induced mPGES-1 induced PGE1 synthesis with an IC₅₀ of 3.29 nM. The results are shown in FIG. 1.

### Human Whole Blood Prostaglandin E Synthase Assay

### Rationale

Whole blood provides a protein and cell-rich milieu for the study of biochemical efficacy of anti-inflammatory compounds such as prostaglandin E synthase inhibitors. To study the inhibitory activities of these compounds, human blood is stimulated with lipopolysaccharide (LPS) for 24 hours to induce mPGES-1 expression. The production of prostaglandin E2 (PGE₂) and thromboxane B2 (TXB2) are measured by EIA at the end of the incubation as readouts for selectivity and effectiveness against mPGES-1-dependent PGE₂ production.

### Methods

Human whole blood assays for mPGES-1 activity reported (Brideau, et al., Inflamm. Res., vol. 45, p. 68, 1996) are performed as described below.

Freshly isolated venous blood from human volunteers is collected in heparinized tubes. These subjects have no apparent inflammatory conditions and have not taken any NSAIDs for at least 7 days prior to blood collection. 250 μl of blood is pre-incubated with 1 ul vehicle (DMSO) or 1 ul of test compound. Bacterial LPS at 100 μg/ml (E. Coli serotype 0111:B4 diluted in 0.1% w/v bovine serum albumin in phosphate buffered saline) is then added and samples are incubated for 24 hours at 37° C. Unstimulated control blood at time zero (no LPS) is used as blank. At the end of the 24 hr incubation, the blood is centrifuged at 3000 rpm for 10 min at 4° C. The plasma is assayed for PGE₂ and TxB₂ using an EIA kit as indicated above.

### Formulation of Test Compounds for Oral Dosage

Test compound was ground and made amorphous using a ball milling system. The compound was placed in an agate jar containing agate balls and spun at high speed for 10 minutes in an apparatus such as the Planetary Micro Mill Pulverisette 7 system. The jar was then opened and 0.5% methocel solution was added to the ground solid. This mixture was spun again at high speed for 10 minutes. The resulting suspension was transferred to a scintillation vial, diluted with the appropriate amount of 0.5% methocel solution, sonicated for 2 minutes and stirred until the suspension was homogeneous. Alternatively, the test compound can be formulated using amorphous material obtained by any suitable chemical or mechanical technique. This amorphous solid is then mixed and stirred for a certain period of time, such as 12 hours, with a suitable vehicle, such as 0.5% methocel with 0.02 to 0.2% of sodium dodecylsulfate, prior to dosage.

An alternate method for making Example 40 is as follows:

### Alternate Example 40

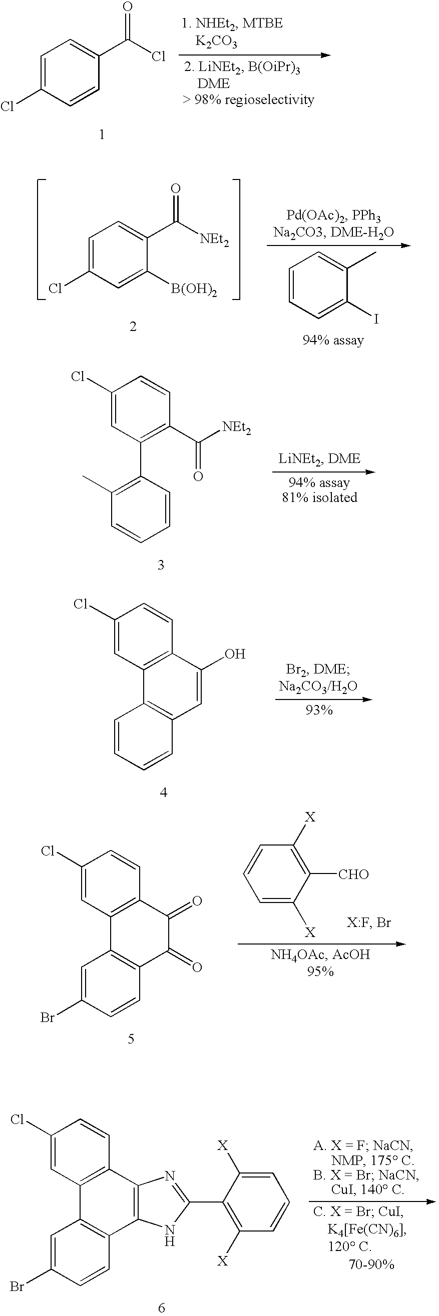

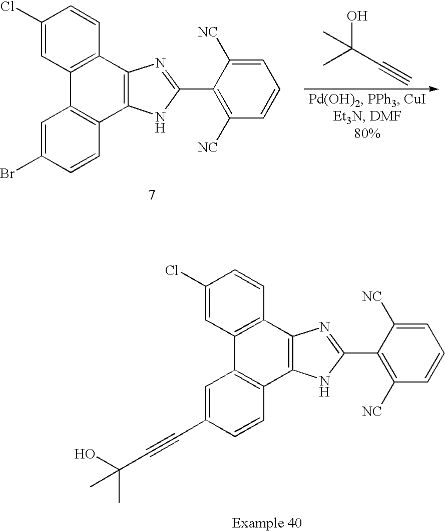

### Experimental Procedure

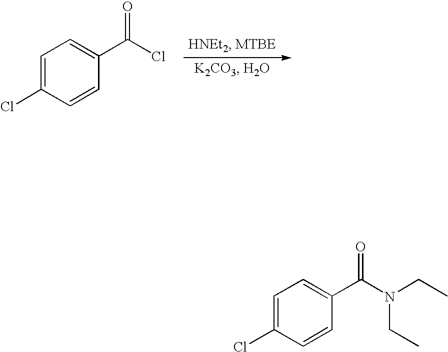

To a round bottom flask was charged potassium carbonate (65 g, 469.7 mmol), H₂O (400 mL), MTBE (800) and diethyl amine (81 mL, 861.1 mmol). p-Chlorobenzoyl chloride (100 mL, 782.8 mmol) was then added over 30 minutes, maintaining the temperature under 25° C. After addition, the phases were separated and the organics washed with brine (200 mL). The solution was then solvent switched to DME to give a crude solution of the amide, which was used directly in the next step.

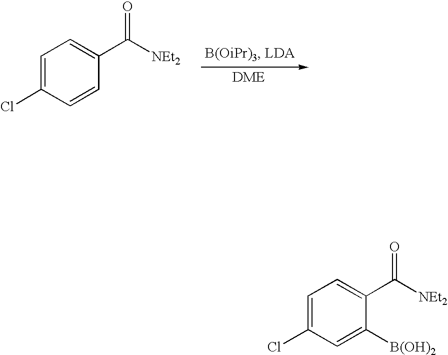

To the crude solution of the amide (10 g, 47.3 mmol) in 7.5 mL/g DME (75 mL) was added triisopropyl borate (19.5 mL, 85.1 mmol) and the resulting solution was cooled to ˜25° C. A freshly prepared 1.45 M solution of lithium diethylamide (45.6 mL, 66.2 mmol) was then added dropwise over 30 minutes. [NOTE: Lithium diethylamide was generated by treatment of diethylamine in THF with a 2.5M solution of n-butyllithium in hexanes, maintaining the temperature below 0° C. during the addition] At the end of addition, the mixture was aged for additional 15 minutes, at which all starting material has been consumed to give the corresponding boronic acid in >98% regioselectivity. The crude solution was then used directly in the next step.

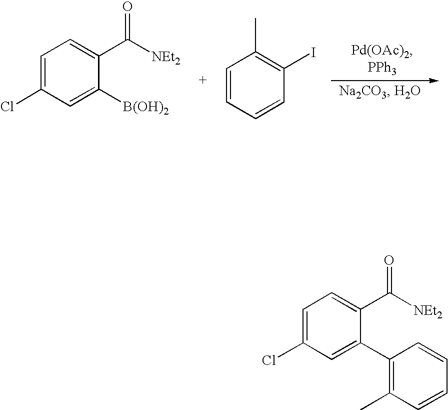

To the crude solution of boronic acid as obtained above was added degassed water (95 mL) at 0° C. and solid Na₂CO₃ (13.5 g, 127.7 mmol). To the resulting suspension was successively added PPh₃ (223 mg, 0.85 mmol), 2-iodotoluene (5.4 mL, 42.6 mmol) and Pd(OAc)₂ (95.5 mg, 0.43 mmol) and the mixture was degassed, heated to 70° C. and aged for 6 hours, at which complete consumption of 2-iodotoluene was typically observed. At the end of reaction, MTBE (75 mL) was added and the resulting slurry was filtered. Sodium chloride was added to the biphasic filtrate to ease the separation and the layers were cut. The organic phase was washed one time with water (20 mL) and brine (2×30 mL). The crude solution was then concentrated, solvent switched to DME and used directly in the next step. Typical assay yield: 90-94%.

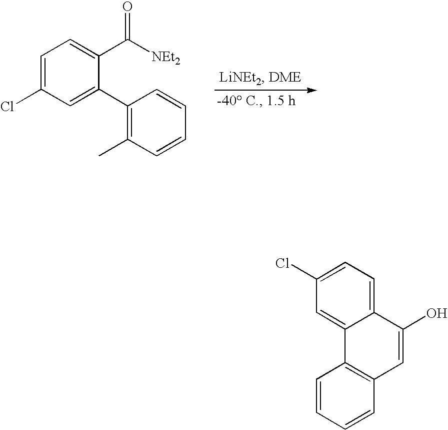

To the crude solution of the amide (13.9 g, 46.2 mmol) in 7.5 mL/g DME (104 mL), kept at −45° C., was added freshly prepared 1.44 M solution of LiNEt₂ in THF (41.7 mL, 60 mmol) over 15 min. The resulting brown solution was aged for 75 minutes, at which complete consumption of starting material was observed by HPLC. MTBE (120 mL) was added followed by slow addition of 6N HCl (30.8 mL, 184.7 mmol). The resulting mixture was allowed to warn to RT and the layers were separated (pH of the aqueous layer should be 2-3). The organic layer was washed one time with H₂O (55 mL), brine (60 mL), concentrated and solvent switched to toluene for crystallization. When approximately 4 mL/g of product in a 3:1 mixture of toluene:DME was obtained, the slurry was refluxed to dissolve all the solid, cooled slowly to 60° C. and treated with 5 mL/g of methyl cyclohexane (crystals are typically formed at 75-80° C.) over 1 hour, while allowing the mixture to cool to RT. The slurry was then concentrated to give a volume of 3.5 mL/g of product and then re-treated with 2 mL/g of methyl cyclohexane over 0.5 hour. The slurry was aged at 0° C. for 0.5 hour, filtered and the wetcake was washed with a cold 3:1 mixture of toluene:methyl cyclohexane, followed by drying under constant flow of N₂. The desired product was obtained as light tan solid in 81% yield.

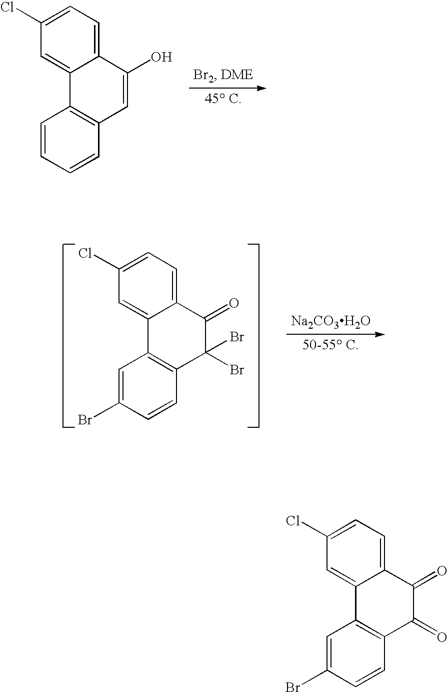

To a solution of chloro-phenanthrole (41 g, 179.8 mmol) in dry DME (600 mL, KF=25 ppm, solution KF=1000 ppm) at 15° C. was added Br₂ (32.3 mL, 629.4 mmol) over 20 minutes, at which a 15° C. exotherm was evident during the addition. The resulting suspension was then warmed to 40-45° C. and aged for 4 hours to give a clear, red solution. A solution of Na₂SO₃ (4.4 g, 36 mmol) in 30 mL of H₂O was added, followed by a solution of Na₂CO₃ (57 g, 539.4 mmol) in 250 mL H₂O. The resulting suspension was warmed to 55° C. and aged for 5 hour, at which a complete hydrolysis was obtained (additional of H₂O might be necessary to re-dissolve precipitated Na₂CO₃). The reaction mixture was then concentrated at 35-40° C. (35-40 torr) to about a third of its volume and the slurry was filtered, washed with H₂O (80-100 mL), followed by 1:1 DME:H₂O (100 mL) and dried under constant flow of N₂. The solid obtained was generally pure enough for the next step; typical yield: 93%.

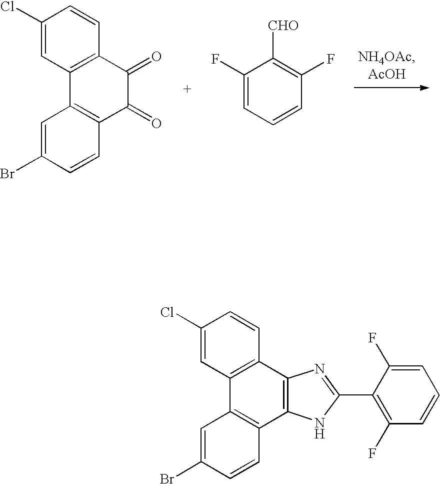

The chlorobromodiketone (4.54 g, 14.12 mmol), difluorobenzaldehyde (1.5 mL, 14.12 mmol), and ammonium acetate (21.77 g, 282.38 mmol) were charged to a 250 mL round bottom three neck flask under nitrogen. Acetic acid (90 mL) was added with stirring, and the slurry was heated to 120° C. for 1 hour. The slurry was then cooled to room temperature and water (90 mL) was added over 30 min. Upon completion of addition of water, the reaction mixture was filtered, washed with water (45 mL), and dried overnight under nitrogen and vacuum to give the acetic acid salt as a yellow solid.

In order to obtain the freebase, the crude product was dissolved in 1:1 THF/MTBE (90 mL) and charged to a 250 mL flask along with 1N NaOH (45 mL). The mixture was then heated to 40° C. for one hour. The phases were cut at 40° C., and the organic layer washed with 1N NaOH (45 mL). The organic layer was then concentrated, solvent switched to MTBE, and brought to a final volume of 45 mL. The reaction mixture was slurried at 35° C. for one hour, cooled to room temperature, filtered, washed with MTBE (23 mL), and dried under nitrogen. The difluoro imidazole freebase (5.97 g) was obtained as a light yellow solid in 95% isolated yield.

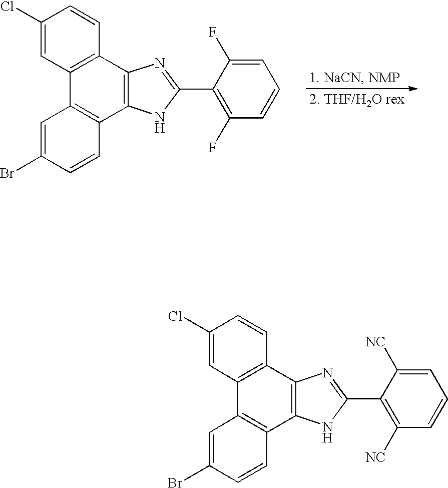

Method A: The difluoroimidazole (6.79 g, 13.39 mmol) and sodium cyanide (3.28 g, 66.95 mmol) were charged to a 500 mL round bottom flask under nitrogen. N-methylpyrrolidone (NMP, 60 mL) was added with stirring, and the slurry was heated to 175° C. for 28 hours. The reaction mixture was then cooled to room temperature. Water (240 mL) was added over 2 hours, and the slurry was allowed to stir for 48 hours. Sodium chloride (36 g) was added to the slurry and it was stirred for additional 2 hours. The slurry was then cooled to 0° C., stirred for 1 hour, filtered, and washed with water (30 mL). The wetcake was then dried under nitrogen to give the desired product as NMP solvate.

The solid was slurried in THF (42 mL, 7.5 mL/g) at 65° C. for 1 hour. The mixture was then cooled to room temperature, followed by addition of water (14 mL, 2.5 mL/g) over 1 hour. The slurry was then concentrated under vacuum, removing 14 mL of solvent and the resulting slurry was filtered. The wetcake was washed with 1:1 THF/H₂O (14 mL), and dried under nitrogen. The desired product (3.83 g) was obtained as THF solvate in 54% isolated yield.

### Method B:

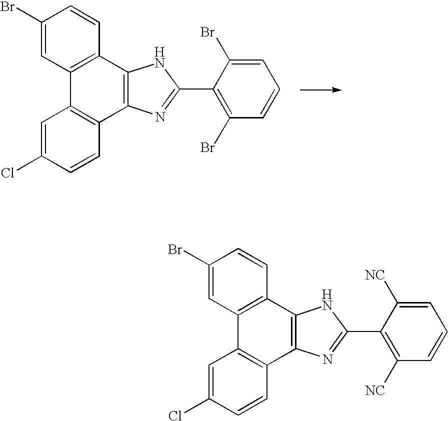

1.0 g of tribromoimidazole freebase (1.8 mmol), 260 mg NaCN (5.3 mmol), 135 mg CuI (0.71 mmol) and 7 mL DMF were combined and degassed, then heated to 120° C. for 45 h. 7 mL of 6:1 water: NH₄OH was added, and the crude product was isolated by filtration. After drying, the material was recrystallized from 1:1 THF:MTBE (16 mL) to afford 870 mg of the dicyano product as the THF solvate (97%).

Method C: tribromoimidazole AcOH salt (1.30 g, 87 wt % as free base, 2 mmol) was treated with K₄[Fe(CN)₆].3H₂O (845 mg, 2 mmol, finely-powdered), CuI (76.2 mg, 0.4 mmol), and 1,2-phenylenediamine (43.3 mg, 0.4 mmol) in DMF (5.7 mL). The reaction mixture was heated to 135° C. for 36 h, diluted with DMF (5.7 mL), and filtered when hot. The solid was washed thoroughly with acetone, and the washes were combined with the filtrate. The organic solution was concentrated to remove acetone, and H₂O (2.8 mL) was added over 15 min at RT. The resulting solid was collected by filtration, washed with H₂O, and to afford brown solid (1.06 g).

The crude solid was then stirred in THF (4 mL) at 60° C. for 1 h and allowed to cool to RT. The resulting solid was collected by filtration, washed with hexane, and dried to afford dicanide THF solvate as off white powder (864 mg, 89.5 wt %).

For Methods B and C above, the tribromoimidazole compound is made following the procedure described above for making the difluoroimidazole compound, but substituting dibromobenzaldehyde for difluorobenzaldehyde.

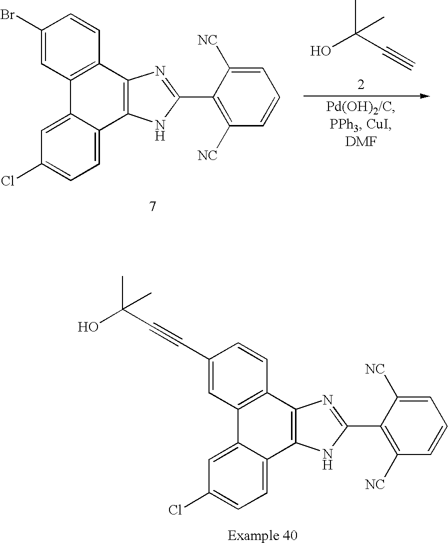

A 7 ml vial, equipped with stir bar and septum screw cap was charged with 6.2 mg of 20 wt % Pd(OH)₂ on carbon containing about 16 wt % water (about 1.0 mg Pd(OH)₂ corrected for solid support and water), 69 mg compound 7, 8 mg triphenylphosphine, and 6 mg copper(I) iodide. The vial was brought into a nitrogen filled glovebox where the remaining nitrogen-purged reaction materials were added. N,N-Dimethylformamide (0.68 mL) was charged followed by 2-methyl-3-butyn-2-ol (0.022 mL) and triethylamine (0.031 mL). The vial was sealed, removed from the glovebox, placed in a heating block equipped with a nitrogen-purged cover attached, and warmed to an external temperature of 52° C. The reaction was agitated with heating for about 17 h. HPLC analysis of the reaction at this time showed about 95% LCAP conversion to Example 40 using an external reference with >99 LCAP conversion of bromide 7 @ 210 nm.

The following examples describe methods for making Example 40 as amorphous material.

### Example A

2 grams of Example 40 solid and 10 ml of dimethyl solfoxide (DMSO) solvent were charged into a glass flask at room temperature. All solids were dissolved. The solution was mixed rapidly with 20 to 30 ml of water (as anti-solvent) using an impinging jet device, similar to the one disclosed in U.S. Pat. No. 5,314,506, granted May 24, 1994, to precipitate Example 40 as amorphous material. The ratio of DMSO to water ratio at the impingement ranges from ½ to ⅓. The resulting slurry was sent to a jacketed crystallizer which contained 30-20 ml of water under agitation. The final DMSO/water ratio is maintained at ⅕. The temperature of the batch was maintained at −5° C. to 5° C. to maintain the stability of amorphous solid of Example 40 in slurry. The slurry was filtered and washed with water at 0° C.-5° C. The wet cake was vacuum dried. The crystallinity of the cake was examined by X-ray diffraction analysis and light microscope. The residual solvent in the cake was analyzed by GC.

The amorphous solid of the light microscopic image are mainly non-birefringent with some birefringent crystals. GC analysis of the amorphous solid shows <0.5 wt % residual DMSO in the solid.

### Example B

To a 125 mL jacketed crystallizer equipped with an IKA-Works rotor/stator homogenizer (model T25 with fine dispersion element) as the agitator, charge 50 mL DI water. Turn on the homogenizer at 9.1 m/s tip speed and adjust the jacket temperature until water temperature in vessel is 0° C. to 2° C. Dissolve 1 gram of Example 40 in 5 ml THF in a separate 50 ml glass flask, then add this solution to the above 125 ml crystallizer over 5 minutes. Following charge, adjust jacket temperature of the above crystallizer to achieve 0-2° C. batch temperature. Filter batch and wash with cold water. Dried sample was analyzed by XRD which confirmed that material was amorphous.

What is claimed is:

1. A method for treating or preventing a neoplasia in a human patient in need of such treatment comprising administering to the patient a compound that inhibits microsomal prostaglandin E synthase-1 in an amount that is effective for treating or preventing the neoplasia.

2. The method according to claim 1 wherein the neoplasia is a benign tumor, growth or polyp.

3. The method according to claim 2 wherein the neoplasia is selected from the group consisting of: squamous cell papilloma, basal cell tumor, transitional cell papilloma, adenoma, gastrinoma, cholangiocellular adenoma, hepatocellular adenoma, renal tubular adenoma, oncocytoma, glomus tumor, melanocytic nevus, fibroma, myxoma, lipoma, leiomyoma, rhabdomyoma, benign teratoma, hemangioma, osteoma, chondroma and meningioma.

4. The method according to claim 1 wherein the neoplasia is a cancerous tumor, growth or polyp.

5. The method according to claim 4 wherein the neoplasia is selected from the group consisting of: squamous cell carcinoma, basal cell carcinoma, transitional cell carcinoma, adenocarcinoma, malignant gastrinoma, cholangiocelleular carcinoma, hepatocellular carcinoma, renal cell carcinoma, malignant melanoma, fibrosarcoma, myxosarcoma, liposarcoma, leimyosarcoma, rhabdomyosarcoma, malignant teratoma, hemangiosarcoma, Kaposi sarcoma, lymphangiosarcoma, osteosarcoma, chondrosarcoma, malignant meningioma, non-Hodgkin lymphoma, Hodgkin lymphoma and leukemia.

6. The method according to claim 1, wherein the neoplasia is cancer selected from the group consisting of: brain cancer, bone cancer, basal cell carcinoma, adenocarcinoma, lip cancer, mouth cancer, esophogeal cancer, small bowel cancer, stomach cancer, colon cancer, rectal cancer, liver cancer, bladder cancer, pancreas cancer, ovary cancer, cervical cancer, lung cancer, breast cancer, head and neck cancer, skin cancer, prostate cancer, gall bladder cancer, thyroid cancer and renal cell carcinoma.

7. The method according to claim 6, wherein the cancer is selected from the group consisting of: colon cancer, esophageal cancer, stomach cancer, breast cancer, head and neck cancer, skin cancer, lung cancer, liver cancer, gall bladder, pancreas cancer, bladder cancer, cervical cancer, prostate cancer, thyroid cancer and brain cancer.

8. The method according to claim 1 wherein the compound that inhibits microsomal prostaglandin E synthase-1 is represented by Formula I 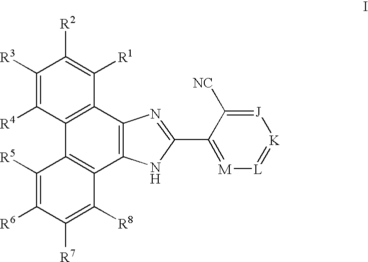
or a prodrug thereof, or a pharmaceutically acceptable salt of said compound or prodrug, wherein:
J is selected from the group consisting of —C(X²)— and —N—,
K is selected from the group consisting of —C(X³)— and —N—,
L is selected from the group consisting of —C(X⁴)— and —N—, and
M is selected from the group consisting of —C(X⁵)— and —N—,
with the proviso that at least one of J, K, L or M is other than —N—;
X², X³, X⁴ and X⁵ are independently selected from the group consisting of: (1) H; (2) —CN; (3) F;
(4) Cl; (5) Br; (6) I; (7) —OH; (8) —N₃; (9) C₁₋₆alkyl, C₂₋₆alkenyl or C₂₋₆alkynyl, wherein one or more of the hydrogen atoms attached to said C₁₋₆alkyl, C₂₋₆alkenyl or C₂₋₆alkynyl may be replaced with a fluoro atom, and said C₁₋₆alkyl, C₂₋₆alkenyl or C₂₋₆alkynyl may be optionally substituted with a hydroxy group; (10) C₁₋₄alkoxy; (11) NR⁹R¹⁰—C(O)—C₁₋₄alkyl-O—; (12) C₁₋₄alkyl-S(O)ₖ—; (13) —NO₂; (14) C₃₋₆cycloalkyl, (15) C₃₋₆cycloalkoxy; (16) phenyl, (17) carboxy; and (18) C₁₋₄alkyl-O—C(O)—;
R¹, R², R³, R⁴, R⁵, R⁶, R⁷ and R⁸ are independently selected from the group consisting of: (1) H; (2) F; (3) Cl; (4) Br; (5) I; (6) —CN; (7) C₁₋₁₀alkyl or C₂₋₁₀alkenyl, wherein one or more of the hydrogen atoms attached to said C₁₋₁₀alkyl or C₂₋₁₀alkenyl may be replaced with a fluoro atom, or two hydrogen on adjacent carbon atoms may be joined together and replaced with —CH₂— to form a cyclopropyl group, or two hydrogen atoms on the same carbon atom may be replaced and joined together to form a spiro C₃₋₆cycloalkyl group, and wherein said C₁₋₁₀alkyl or C₂₋₁₀alkenyl may be optionally substituted with one to three substituents independently selected from the group consisting of: —OH, acetyl, methoxy, ethenyl, R¹¹—O—C(O)—, R³⁵—N(R³⁶)—, R³⁷—N(R³⁸)—C(O)—, cyclopropyl, pyrrolyl, imidiazolyl, pyridyl and phenyl, said pyrrolyl, imidiazolyl, pyridyl and phenyl optionally substituted with C₁₋₄alkyl or mono-hydroxy substituted C₁₋₄alkyl; (8) C₃₋₆cycloalkyl; (9) R¹²—O—; (10) R¹³—S(O)ₖ—, (11) R¹⁴—S(O)ₖ—N(R¹⁵)—; (12) R¹⁶—C(O)—; (13) R¹⁷—N(R¹⁸)—; (14) R¹⁹—N(R²⁰)—C(O)—; (15) R²¹—N(R²²)—S(O)ₖ—; (16) R²³—C(O)—N(R²⁴)—; (17) Z-C≡C; (18) —(CH₃)C═N—OH or —(CH₃)C═N—OCH₃; (19) R³⁴—O—C(O)—; (20) R³⁹—C(O)—O—; and (21) phenyl, naphthyl, pyridyl, pyradazinyl, pyrimidinyl, pyrazinyl, pyrrolyl, pyrazolyl, imidazolyl, triazolyl, tetrazolyl, oxazolyl, isoxazolyl, oxadiazolyl, thienyl or furyl, each optionally substituted with a substituent independently selected from the group consisting of: F, Cl, Br, I, C₁₋₄alkyl, phenyl, methylsulfonyl, methylsulfonylamino, R²⁵—O—C(O)— and R²⁶—N(R²⁷)—, said C₁₋₄alkyl optionally substituted with 1 to 3 groups independently selected from halo and hydroxy;
each Z is independently selected from the group consisting of: (1) H; (2) C₁₋₆alkyl, wherein one or more of the hydrogen atoms attached to said C₁₋₆alkyl may be replaced with a fluoro atom, and wherein
C₁₋₆alkyl is optionally substituted with one to three substituents independently selected from: hydroxy, methoxy, cyclopropyl, phenyl, pyridyl, pyrrolyl, R²⁸—N(R²⁹)— and R³⁰—O—C(O)—; (3) —(CH₃)C═N—OH or —(CH₃)C═N—OCH₃; (4) R³¹—C(O)—; (5) phenyl; (6) pyridyl or the N-oxide thereof; (7) C₃₋₆cycloalkyl, optionally substituted with hydroxy; (8) tetrahydropyranyl, optionally substituted with hydroxy; and (9) a five-membered aromatic heterocycle containing 1 to 3 atoms independently selected from O, N or S and optionally substituted with methyl;
each R⁹, R¹⁰, R¹⁵, R²⁴ and R³² is independently selected from the group consisting of: (1) H; and
(2) C₁₋₄alkyl;
each R¹¹, R¹², R¹³, R¹⁴, R¹⁶, R²³, R²⁵, R³⁰, R³¹, R³⁴ and R³⁹ is independently selected from the group consisting of: (1) H; (2) C₁₋₄alkyl, (3) C₃₋₆cycloalkyl; (4) C₃₋₆cycloalkyl-C₁₋₄alkyl- (5) phenyl, (6) benzyl; and (7) pyridyl; said C₁₋₄alkyl, C₃₋₆cycloalkyl, C₃₋₆cycloalkyl-C₁₋₄alkyl-, phenyl, benzyl and pyridyl may each be optionally substituted with 1 to 3 substituents independently selected from the group consisting of: OH, F, Cl, Br and I, and wherein said C₁₋₄alkyl may be further substituted with oxo or methoxy or both;
each R¹⁷, R¹⁸, R¹⁹, R²⁰, R²¹, R²², R²⁶, R²⁷, R²⁸, R²⁹, R³⁵, R³⁶, R³⁷ and R³⁸ is independently selected from the group consisting of: (1) H; (2) C₁₋₆alkyl; (3) C₁₋₆alkoxy; (4) OH and (5) benzyl or 1-phenylethyl; and R¹⁷ and R¹⁸, R¹⁹ and R²⁰, R²¹ and R²², R²⁶ and R²⁷, and R²⁸ and R²⁹, R³⁵ and R³⁶, and R³⁷ and R³⁸ may be joined together with the nitrogen atom to which they are attached to form a monocyclic ring of 5 or 6 carbon atoms, optionally containing one or two atoms independently selected from —O—, —S(O)ₖ— and —N(R³²)—; and
each k is independently 0, 1 or 2.

9. The method according to claim 8 wherein the compound that inhibits microsomal prostaglandin E synthase-1 is represented by Formula I 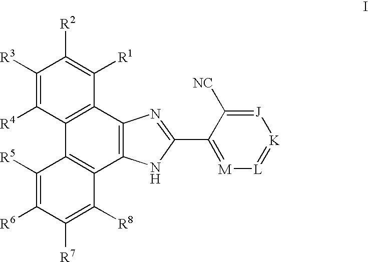
or a prodrug thereof, or a pharmaceutically acceptable salt of said compound or prodrug, wherein:
J is selected from the group consisting of —C(X²)— and —N—,
K is selected from the group consisting of —C(X³)— and —N—,
L is selected from the group consisting of —C(X⁴)— and —N—, and
M is selected from the group consisting of —C(X⁵)— and —N—,
with the proviso that at least one of J, K, L or M is other than —N—;
X², X³, X⁴ and X⁵ are independently selected from the group consisting of: (1) H; (2) —CN; (3) F;
(4) Cl; (5) Br; (6) I; (7) —OH; (8) —N₃; (9) C₁₋₆alkyl, C₂₋₆alkenyl or C₂₋₆alkynyl, wherein one or more of the hydrogen atoms attached to said C₁₋₆alkyl, C₂₋₆alkenyl or C₂₋₆alkynyl may be replaced with a fluoro atom, and said C₁₋₆alkyl, C₂₋₆alkenyl or C₂₋₆alkynyl may be optionally substituted with a hydroxy group; (10) C₁₋₄alkoxy; (11) NR⁹R¹⁰—C(O)—C₁₋₄alkyl-O—; (12) C₁₋₄alkyl-S(O)ₖ—; (13) —NO₂; (14) C₃₋₆cycloalkyl, (15) C₃₋₆cycloalkoxy; (16) phenyl, (17) carboxy; and (18) C₁₋₄alkyl-O—C(O)—;
R¹, R², R³, R⁴, R⁵, R⁶, R⁷ and R⁸ are independently selected from the group consisting of: (1) H; (2) F; (3) Cl; (4) Br; (5) I; (6) —CN; (7) C₁₋₆alkyl or C₂₋₆alkenyl, wherein one or more of the hydrogen atoms attached to said C₁₋₆alkyl or C₂₋₆alkenyl may be replaced with a fluoro atom, and wherein said C₁₋₆alkyl or C₂₋₆alkenyl may be optionally substituted with one to three substituents independently selected from the group consisting of: —OH, methoxy, R¹¹—O—C(O)—, cyclopropyl, pyridyl and phenyl; (8) C₃₋₆cycloalkyl; (9) R¹²—O—; (10) R¹³—S(O)ₖ—, (11) R¹⁴—S(O)ₖ—N(R¹⁵)—; (12) R¹⁶—C(O)—; (13) R¹⁷—N(R¹⁸)—; (14) R¹⁹—N(R²⁰)—C(O)—; (15) R²¹—N(R²²)—S(O)ₖ—; (16) R²³—C(O)—N(R²⁴)—; (17) Z-C≡C;
(18) (CH₃)C═N—OH or —(CH₃)C═N—OCH₃; and (19) phenyl, naphthyl, pyridyl, pyradazinyl, pyrimidinyl, pyrazinyl, pyrrolyl, pyrazolyl, imidazolyl, triazolyl, tetrazolyl, oxazolyl, isoxazolyl, oxadiazolyl, thienyl or furyl, each optionally substituted with a substituent independently selected from the group consisting of: F, Cl, Br, I, C₁₋₄alkyl, phenyl, methylsulfonyl, methylsulfonylamino, R²⁵—O—C(O)— and R²⁶—N(R²⁷)—, said C₁₋₄alkyl optionally substituted with 1 to 3 groups independently selected from halo and hydroxy;
each Z is independently selected from the group consisting of: (1) H; (2) C₁₋₆alkyl, wherein one or more of the hydrogen atoms attached to said C₁₋₆alkyl may be replaced with a fluoro atom, and wherein
C₁₋₆alkyl is optionally substituted with one to three substituents independently selected from: hydroxy, methoxy, cyclopropyl, phenyl, pyridyl, pyrrolyl, R²⁸—N(R²⁹)— and R³⁰—O—C(O)—; (3) —(CH₃)C═N—OH or —(CH₃)C═N—OCH₃; (4) R³¹—C(O)—; (5) phenyl; (6) pyridyl or the N-oxide thereof; (7) C₃₋₆cycloalkyl, optionally substituted with hydroxy; (8) tetrahydropyranyl, optionally substituted with hydroxy; and (9) a five-membered aromatic heterocycle containing 1 to 3 atoms independently selected from O, N or S and optionally substituted with methyl;
each R⁹, R¹⁰, R¹⁵, R²⁴ and R³² is independently selected from the group consisting of: (1) H; and
(2) C₁₋₄alkyl;
each R¹¹, R¹², R¹³, R¹⁴, R¹⁶, R²³, R²⁵, R³⁰ and R³¹ is independently selected from the group consisting of: (1) H; (2) C₁₋₄alkyl, (3) C₃₋₆cycloalkyl; (4) phenyl, (5) benzyl; and (6) pyridyl; said C₁₋₄alkyl, C₃₋₆cycloalkyl, phenyl, benzyl and pyridyl may each be optionally substituted with 1 to 3 substituents independently selected from the group consisting of: OH, F, Cl, Br and I;
each R¹⁷, R¹⁸, R¹⁹, R²⁰, R²¹, R²², R²⁶, R²⁷, R²⁸ and R²⁹ is independently selected from the group consisting of: (1) H; (2) C₁₋₆alkyl; (3) C₁₋₆alkoxy; (4) OH and (5) benzyl or 1-phenylethyl; and R¹⁷ and R¹⁸, R¹⁹ and R²⁰, R²¹ and R²², R²⁶ and R²⁷, and R²⁸ and R²⁹ may be joined together with the nitrogen atom to which they are attached to form a monocyclic ring of 5 or 6 carbon atoms, optionally containing one or two atoms independently selected from —O—, —S(O)ₖ— and —N(R³²)—; and
each k is independently 0, 1 or 2.

10. The method according to claim 8 wherein the compound that inhibits microsomal prostaglandin E synthase-1 is represented by Formula A 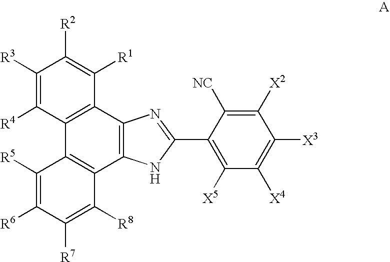
or a prodrug thereof, or a pharmaceutically acceptable salt of said compound or prodrug.

11. The method according to claim 10 wherein the compound that inhibits microsomal prostaglandin E synthase-1 is represented by Formula A wherein:
X², X³, X⁴ and X⁵ are independently selected from the group consisting of: (1) H; (2) —CN; (3) F;
(4) Cl; (5) Br; and (6) I.

12. The method according to claim 10 wherein the compound that inhibits microsomal prostaglandin E synthase-1 is represented by Formula A wherein X², X³ and X⁴ are H, and X⁵ is other than H.

13. The method according to claim 12 wherein the compound that inhibits microsomal prostaglandin E synthase-1 is represented by Formula A wherein X⁵ is —CN.

14. The method according to claim 10 wherein the compound that inhibits microsomal prostaglandin E synthase-1 is represented by Formula A wherein at least one of R¹ or R⁸ is other than H.

15. The method according to claim 10 wherein the compound that inhibits microsomal prostaglandin E synthase-1 is represented by Formula A wherein at least one of R² or R⁷ is other than H.

16. The method according to claim 10 wherein the compound that inhibits microsomal prostaglandin E synthase-1 is represented by Formula A wherein at least one of R⁴ or R⁵ is other than H.

17. The method according to claim 10 wherein the compound that inhibits microsomal prostaglandin E synthase-1 is represented by Formula A wherein:
at least one of R³ or R⁶ is other than H; and
R¹, R², R⁴, R⁵, R⁷ and R⁸ are H.

18. The method according to claim 17 wherein the compound that inhibits microsomal prostaglandin E synthase-1 is represented by Formula A wherein R³ and R⁶ are both other than H.

19. The method according to claim 18 wherein the compound that inhibits microsomal prostaglandin E synthase-1 is represented by Formula A wherein:
one of R³ or R⁶ is independently selected from the group consisting of: F, Cl, Br, and I; and
the other of R³ or R⁶ is Z-C≡C.

20. The method according to claim 17 wherein the compound that inhibits microsomal prostaglandin E synthase-1 is represented by Formula A wherein: R³ and R⁶ are independently selected from the group consisting of: hydrogen, fluoro, chloro, bromo, iodo, cyano, methyl, ethyl, vinyl, cyclopropyl, —CO₂i-Pr, —CO₂CH₃, —SO₂CF₃, 3-pyridyl, acetyl, 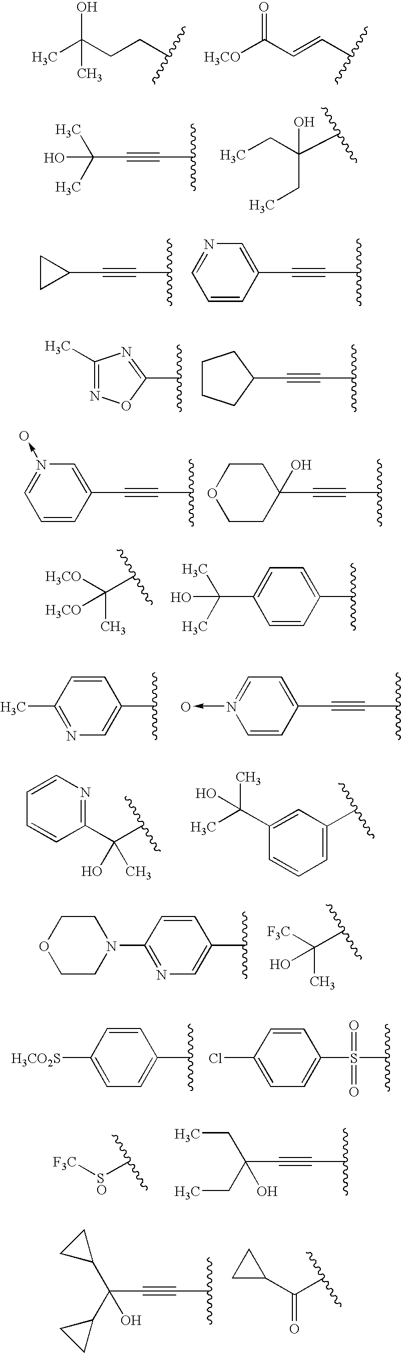 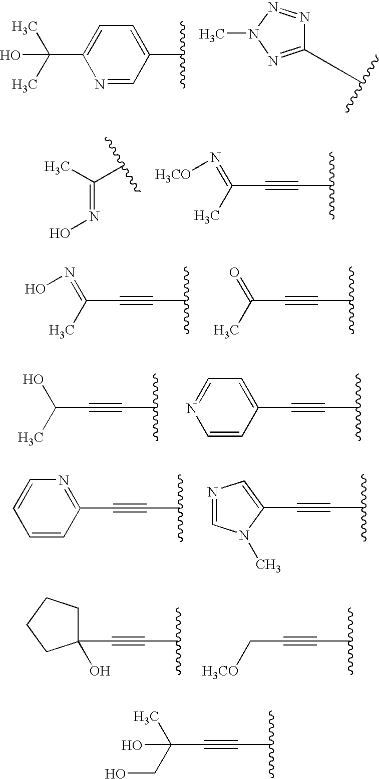
with the proviso that at least one of R³ or R⁶ is other than H.

21. The method according to claim 9 wherein the compound that inhibits microsomal prostaglandin E synthase-1 is represented by Formula B: 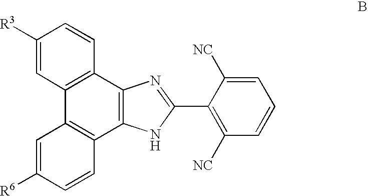
or a prodrug thereof, or a pharmaceutically acceptable salt of said compound or prodrug.

22. The method according to claim 21 wherein the compound that inhibits microsomal prostaglandin E synthase-1 is represented by Formula B wherein:
one of R³ or R⁶ is independently selected from the group consisting of: F, Cl, Br, and I; and
the other of R³ or R⁶ is Z-C≡C.

23. The method according to claim 8 wherein the compound that inhibits microsomal prostaglandin E synthase-1 is a prodrug represented by Formula C 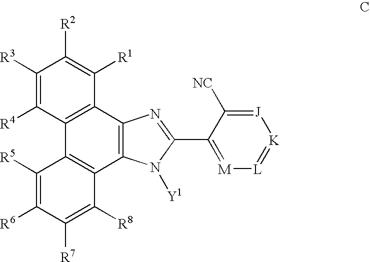
or a pharmaceutically acceptable salt thereof, wherein:
Y¹ is selected from the group consisting of: (1) C₁₋₆alkyl; (2) PO₄—C₁₋₄alkyl-; (3) C₁₋₄alkyl-C(O)—O—CH₂—, wherein the C₄alkyl portion is optionally substituted with R³³—O—C(O)—; and (4) C₁₋₄alkyl-O—C(O)—; and
R³³ is selected from the group consisting of: (1) H; (2) C₁₋₄alkyl, (3) C₃₋₆cycloalkyl; (4) phenyl; (5) benzyl; and (6) pyridyl; said C₁₋₄alkyl, C₃₋₆cycloalkyl, phenyl, benzyl and pyridyl may each be optionally substituted with 1 to 3 substituents independently selected from the group consisting of: OH, F, Cl, Br and I.

24. The method according to claim 8 wherein the compound that inhibits microsomal prostaglandin E synthase-1 is selected from one of the following tables:

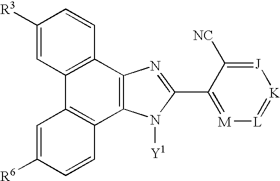

| Ex | R³/R⁶ | R⁶/R³ | J | K | L | M | Y¹ |
|---|---|---|---|---|---|---|---|
| 1 | Cl | Br | CH | CH | CH | CF | H |
| 2 | H | H | CH | CH | CH | CH | H |
| 3 | CN | 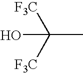 | CH | CH | CH | CF | H |
| 4 | Cl | 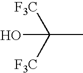 | CH | CH | CH | CF | H |
| 5 | Cl | H | CH | CH | CH | CF | H |
| 6 | CN | H | CH | CH | CH | CF | H |
| 7 | CN | 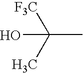 | CH | CH | CH | CF | H |
| 8 | Cl | 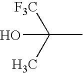 | CH | CH | CH | CF | H |
| 9 | Br | Br | CH | CH | CH | CF | H |
| 10 | H | H | CH | CH | CH | CCl | H |
| 11 | H | H | CH | CH | CH | CCN | H |
| 12 | 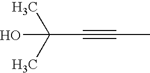 | Br | CH | CH | CH | CF | H |
| 13 | 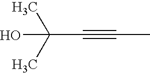 | 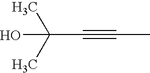 | CH | CH | CH | CF | H |
| 14 | 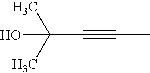 | Cl | CH | CH | CH | CF | H |
| 15 | 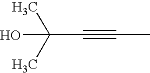 | I | CH | CH | CH | CF | H |
| 16 | H | H | CH | CH | CH | CBr | H |
| 17 | H | H | CH | CH | CH | CF | H |
| 18 | H | H | CH | N | CH | CCl | H |
| 19 | 3-pyridyl | 3-pyridyl | CH | CH | CH | CF | H |
| 20 | Cl | 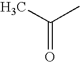 | CH | CH | CH | CF | H |
| 21 | Cl | 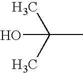 | CH | CH | CH | CF | H |
| 22 | 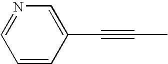 | Br | CH | CH | CH | CF | H |
| 23 | Cl | H | CH | N | CH | CCN | H |
| 24 | H | H | CH | N | CH | CCN | H |
| 25 | Cl | H | CH | CH | CH | CCN | H |
| 26 | H | H | CH | N | CH | CH | H |
| 27 | 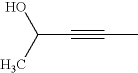 | Br | CH | CH | CH | CF | H |
| 28 | 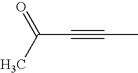 | Br | CH | CH | CH | CF | H |
| 29 | 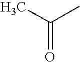 | 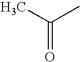 | CH | CH | CH | CF | H |
| 30 | 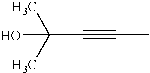 | 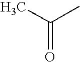 | CH | CH | CH | CF | H |
| 31 | H | H | N | CH | CH | N | H |
| 32 | H | H | N | CH | CH | CH | H |
| 33 | Br | 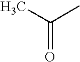 | CH | CH | CH | CF | H |
| 34 | I | I | CH | CH | CH | CF | H |
| 35 | Br | 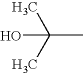 | CH | CH | CH | CF | H |
| 36 | Br | Cl | CH | CH | CH | CCN | H |
| 37 | Cl | 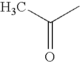 | CH | CH | CH | CBr | H |
| 38 | Cl | 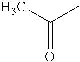 | CH | CH | CH | CCN | H |
| 39 | I | I | CH | CH | CH | CCN | H |
| 40 | 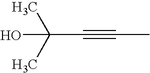 | Cl | CH | CH | CH | CCN | H |
| 41 | Cl | 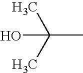 | CH | CH | CH | CCN | H |
| 42 | 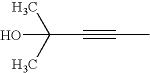 | I | CH | CH | CH | CCN | H |
| 43 | 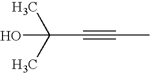 | 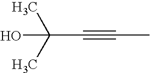 | CH | CH | CH | CCN | H |
| 44 | H | H | CH | CH | CH | CCN | CO₂Et |
| 45 | H | H | CH | CH | CH | CCN | 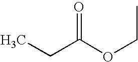 |
| 46 | 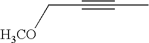 | Cl | CH | CH | CH | CCN | H |
| 47 | 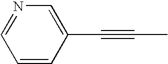 | Cl | CH | CH | CH | CCN | H |
| 48 | 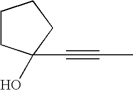 | Cl | CH | CH | CH | CCN | H |
| 49 | 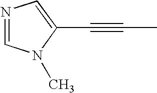 | Cl | CH | CH | CH | CCN | H |
| 50 | 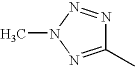 | Cl | CH | CH | CH | CCN | H |
| 51 | Cl | 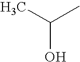 | CH | CH | CH | CCN | H |
| 52 | 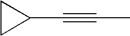 | Cl | CH | CH | CH | CCN | H |
| 53 | 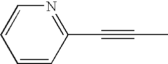 | Cl | CH | CH | CH | CCN | H |
| 54 | 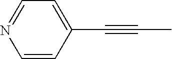 | Cl | CH | CH | CH | CCN | H |
| 55 | 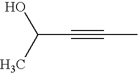 | Cl | CH | CH | CH | CCN | H |
| 56 | 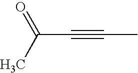 | Cl | CH | CH | CH | CCN | H |
| 57 | 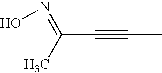 | Cl | CH | CH | CH | CCN | H |
| 58 | 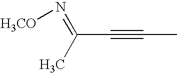 | Cl | CH | CH | CH | CCN | H |
| 59 | H | H | CH | CH | CH | CCN | 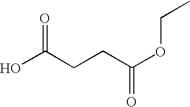 |
| 60 | H | H | CH | CH | CH | CCN | H₂PO₄CH₂ |
| 61 | 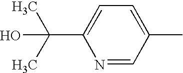 | Cl | CH | CH | CH | CCN | H |
| 62 | Cl | SO₂CH₃ | CH | CH | CH | CCN | H |
| 63 | Cl | 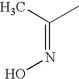 | CH | CH | CH | CCN | H |
| 64 | Br | H | CH | CH | CH | CCN | H |
| 65 | Cl | 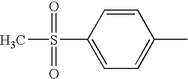 | CH | CH | CH | CCN | H |
| 66 | I | H | CH | CH | CH | CCN | H |
| 67 | CN | H | CH | CH | CH | CCN | H |
| 68 | cyclopropyl | Cl | CH | CH | CH | CCN | H |
| 69 | 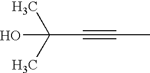 | 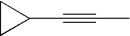 | CH | CH | CH | CCN | H |
| 70 | Cl | F | CH | CH | CH | CCN | H |
| 71 | Cl | 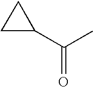 | CH | CH | CH | CCN | H |
| 72 | Cl | 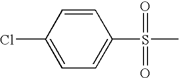 | CH | CH | CH | CCN | H |
| 73 | vinyl | H | CH | CH | CH | CCN | H |
| 74 | ethyl | H | CH | CH | CH | CCN | H |
| 75 | cyclopropyl | H | CH | CH | CH | CCN | H |
| 76 | Cl | 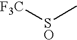 | CH | CH | CH | CBr | H |
| 77 | Cl | 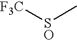 | CH | CH | CH | CCN | H |
| 78 | Cl | SO₂CF₃ | CH | CH | CH | CCN | H |
| 79 | 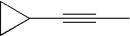 | H | CH | CH | CH | CCN | H |
| 80 | Cl | 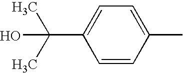 | CH | CH | CH | CCN | H |
| 81 | 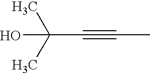 | Br | CH | CH | CH | CCN | H |
| 82 | Cl | 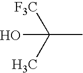 | CH | CH | CH | CCN | H |
| 83 | 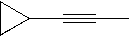 | 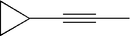 | CH | CH | CH | CCN | H |
| 84 | 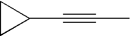 | 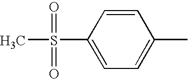 | CH | CH | CH | CCN | H |
| 85 | 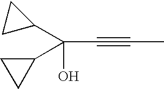 | Cl | CH | CH | CH | CCN | H |
| 86 | 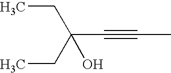 | Cl | CH | CH | CH | CCN | H |
| 87 | Br | 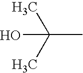 | CH | CH | CH | CCN | H |
| 88 | 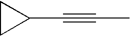 | 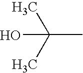 | CH | CH | CH | CCN | H |
| 89 | 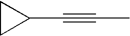 | CN | CH | CH | CH | CCN | H |
| 90 | 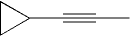 | CO₂CH₃ | CH | CH | CH | CCN | H |
| 91 | 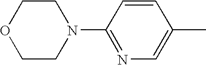 | Cl | CH | CH | CH | CCN | H |
| 92 | Cl | CN | CH | CH | CH | CCN | H |
| 93 | Cl | 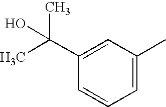 | CH | CH | CH | CCN | H |
| 94 | Br | 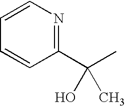 | CH | CH | CH | CCN | H |
| 95 | 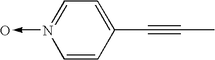 | Cl | CH | CH | CH | CCN | H |
| 96 | 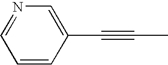 | 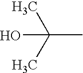 | CH | CH | CH | CCN | H |
| 97 | 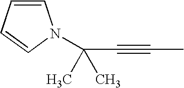 | Cl | CH | CH | CH | CCN | H |
| 98 | 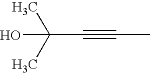 | Br | CH | CH | CH | CCl | H |
| 99 | 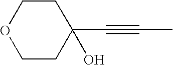 | Br | CH | CH | CH | CCl | H |
| 100 | Cl | CO₂i-Pr | CH | CH | CH | CCN | H |
| 101 | Cl | 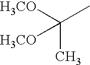 | CH | CH | CH | CF | H |
| 102 | 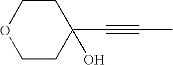 | Br | CH | CH | CH | CCN | H |
| 103 | 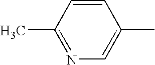 | Cl | CH | CH | CH | CCN | H |
| 104 | Br | 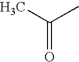 | CH | CH | CH | CCN | H |
| 105 | 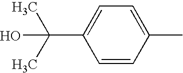 | Cl | CH | CH | CH | CCl | H |
| 106 | Br | 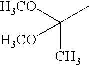 | CH | CH | CH | CCN | H |
| 107 | 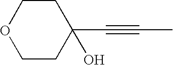 | Cl | CH | CH | CH | CCl | H |
| 108 | 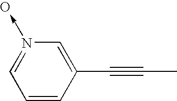 | Cl | CH | CH | CH | CCN | H |
| 109 | 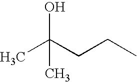 | Br | CH | CH | CH | CCN | H |
| 110 | 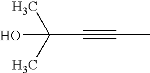 | Cl | CH | CH | CH | CCl | H |
| 111 | 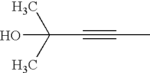 | 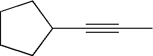 | CH | CH | CH | CCN | H |
| 112 | 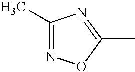 | Br | CH | CH | CH | CCN | H |
| 113 | 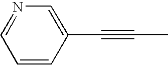 | 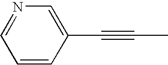 | CH | CH | CH | CCN | H |
| 114 | Et | 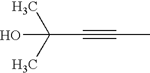 | CH | CH | CH | CCN | H |
| 115 | 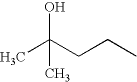 | 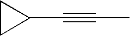 | CH | CH | CH | CCN | H |
| 116 | Br | 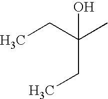 | CH | CH | CH | CCN | H |
| 117 | 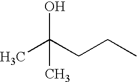 | Cl | CH | CH | CH | CCN | H |
| 118 | Br | CH₃ | CH | CH | CH | CCN | H |
| 119 | 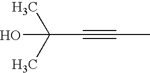 | CH₃ | CH | CH | CH | CCN | H |
| 120 | 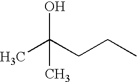 | CH₃ | CH | CH | CH | CCN | H |
| 121 | 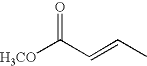 | Cl | CH | CH | CH | CCN | H |
| 122 | 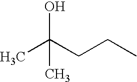 | H | CH | CH | CH | CCN | H |
| 123 | 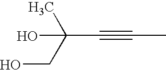 | Cl | CH | CH | CH | CCN | H |
| 124 | 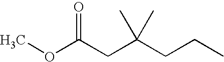 | Cl | CH | CH | CH | CCN | H |
| 125 | 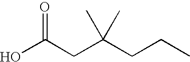 | Cl | CH | CH | CH | CCN | H |
| 126 | 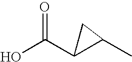 | Cl | CH | CH | CH | CCN | H |
| 127 | 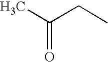 | Cl | CH | CH | CH | CCN | H |
| 128 | 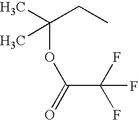 | Cl | CH | CH | CH | CCN | H |
| 129 | 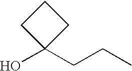 | Cl | CH | CH | CH | CCN | H |
| 130 | 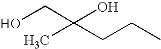 | Cl | CH | CH | CH | CCN | H |
| 131 | 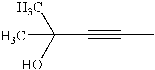 | Cl | CH | F | CH | CCN | H |
| 132 | 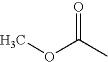 | 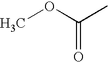 | CH | CH | CH | CCN | H |
| 133 | 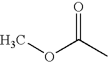 | 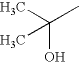 | CH | CH | CH | CCN | H |
| 134 | 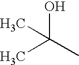 | 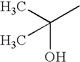 | CH | CH | CH | CCN | H |
| 135 | 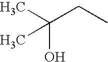 | Cl | CH | CH | CH | CCN | H |
| 136 | Br | Cl | CH | OH | CH | CCN | H |
| 137 | 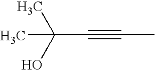 | Cl | CH | OH | CH | CCN | H |
| 138 | 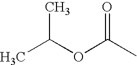 | 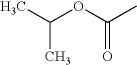 | CH | CH | CH | CCN | H |
| 139 | 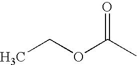 | 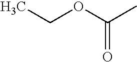 | CH | CH | CH | CCN | H |
| 140 | 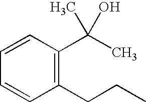 | 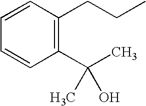 | CH | CH | CH | CCN | H |
| 141 | 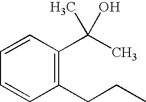 | Br | CH | CH | CH | CCN | H |
| 142 | 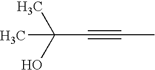 | Cl | CH | Cl | CH | CCN | H |
| 143 | 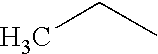 | 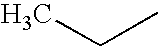 | CH | CH | CH | CCN | H |
| 144 | 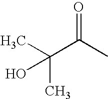 | Cl | CH | CH | CH | CCN | H |
| 145 | Br | 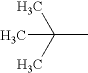 | CH | CH | CH | CCN | H |
| 146 | 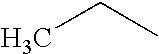 | 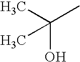 | CH | CH | CH | CCN | H |
| 147 | 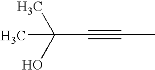 | 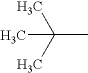 | CH | CH | CH | CCN | H |
| 148 | 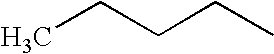 | 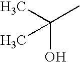 | CH | CH | CH | CCN | H |
| 149 | 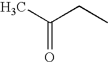 | 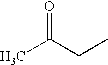 | CH | CH | CH | CCN | H |
| 150 | 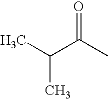 | Cl | CH | F | CH | CCN | H |
| 151 | 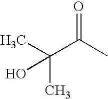 | Cl | CH | F | CH | CCN | H |
| 152 | 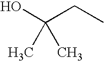 | Cl | CH | F | CH | CCN | H |
| 153 | 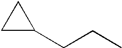 | 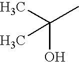 | CH | CH | CH | CCN | H |
| 154 | 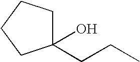 | Cl | CH | CH | CH | CCN | H |
| 155 | 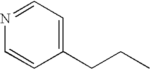 | Cl | CH | CH | CH | CCN | H |
| 156 | Br | 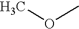 | CH | CH | CH | CCN | H |
| 157 | 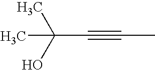 | 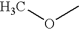 | CH | CH | CH | CCN | H |
| 158 | 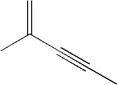 | Cl | CH | CH | CH | CCN | H |
| 159 | 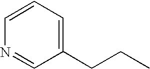 | 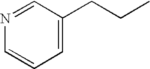 | CH | CH | CH | CCN | H |
| 160 | 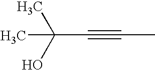 | 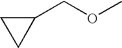 | CH | CH | CH | CCN | H |
| 161 | 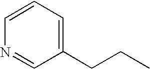 | 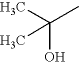 | CH | CH | CH | CCN | H |
| 162 | 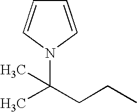 | Cl | CH | CH | CH | CCN | H |
| 163 | 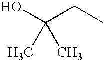 | 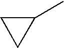 | CH | CH | CH | CCN | H |
| 164 | 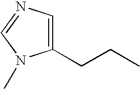 | Cl | CH | CH | CH | CCN | H |
| 165 | 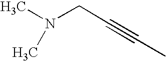 | Cl | CH | CH | CH | CCN | H |
| 166 | 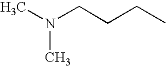 | Cl | CH | CH | CH | CCN | H |
| 167 | 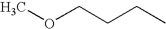 | Cl | CH | CH | CH | CCN | H |
| 168 | 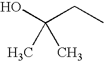 | 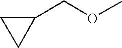 | CH | CH | CH | CCN | H |
| 169 | 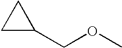 | 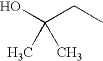 | CH | F | CH | CCN | H |
| 170 | 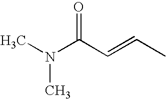 | Cl | CH | CH | CH | CCN | H |
| 171 | 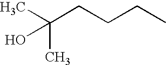 | Cl | CH | CH | CH | CCN | H |
| 172 | 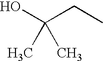 | 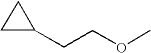 | CH | F | CH | CCN | H |
| 173 | Br | 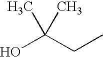 | CH | CH | CH | CCN | H |
| 174 | 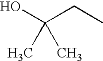 | 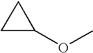 | CH | CH | CH | CCN | H |
| 175 | 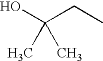 | 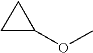 | CH | F | CH | CCN | H |
| 176 | 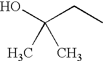 | 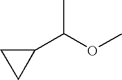 | CH | CH | CH | CCN | H |
| 177 | 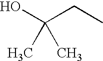 | 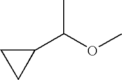 | CH | F | CH | CCN | H |
| 178 | OH | Cl | CH | CH | CH | CCN | H |
| 179 | Cl | 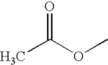 | CH | CH | CH | CCN | H |
| 180 | 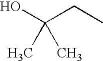 | 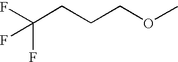 | CH | CH | CH | CCN | H |
| 181 | Cl | 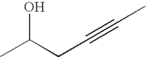 | CH | CH | CH | CCN | H |
| 182 | 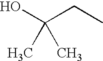 | 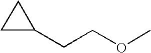 | CH | CH | CH | CCN | H |
| 183 | 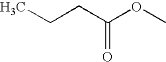 | Cl | CH | CH | CH | CCN | H |
| 184 | Cl | 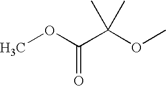 | CH | CH | CH | CCN | H |
| 185 | Cl | 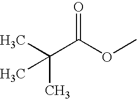 | CH | CH | CH | CCN | H |
| 186 | 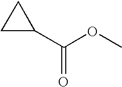 | Cl | CH | CH | CH | CCN | H |
| 187 | Br | Cl | CH | 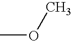 | CH | CCN | H |
| 188 | 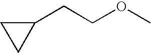 | 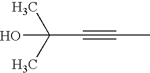 | CH | CF | CH | CCN | H |
| 189 | Cl | Br | CH | N | CH | CCN | H |
| 190 | 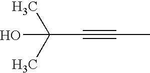 | Cl | CH | N | CH | CCN | H |

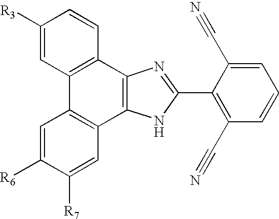
| EX | R3 | R6 | R7 |
|---|---|---|---|
| 191 | 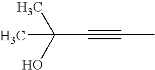 | Cl | 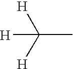 |
| 192 | Cl | H | Br |
| 193 | Cl | H | 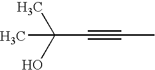 |
| 194 | Cl | H | 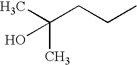 |

or a pharmaceutically acceptable salt of any of the above compounds.

25. The method according to claim 17 wherein the compound that inhibits microsomal prostaglandin E synthase-1 is represented by Formula A wherein: R³ and R⁶ are independently selected from the group consisting of: hydrogen, fluoro, chloro, bromo, iodo, cyano, methyl, methoxy, ethyl, vinyl, cyclopropyl, propyl, butyl, —CO₂i-Pr, —CO₂CH₃, —SO₂CF₃, 3-pyridyl, acetyl, 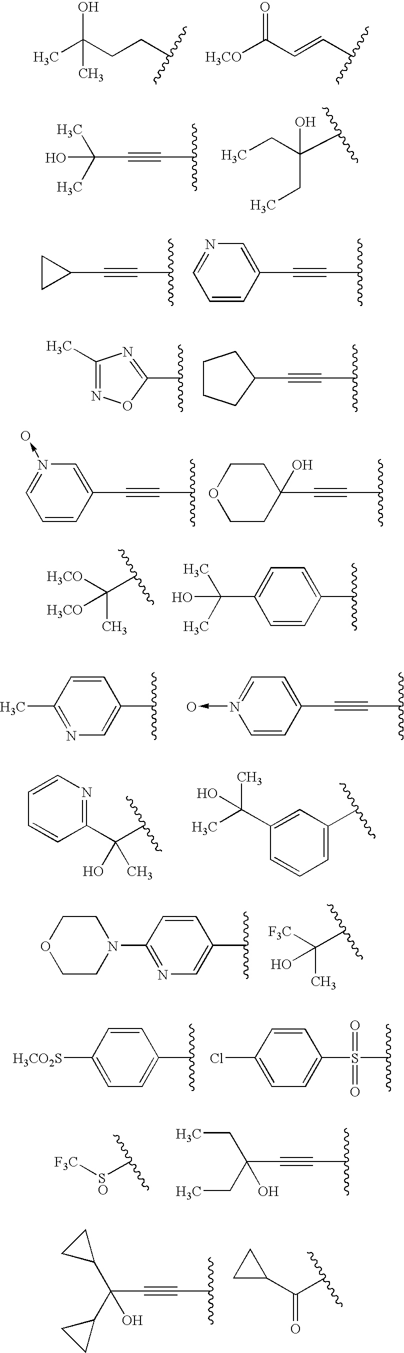 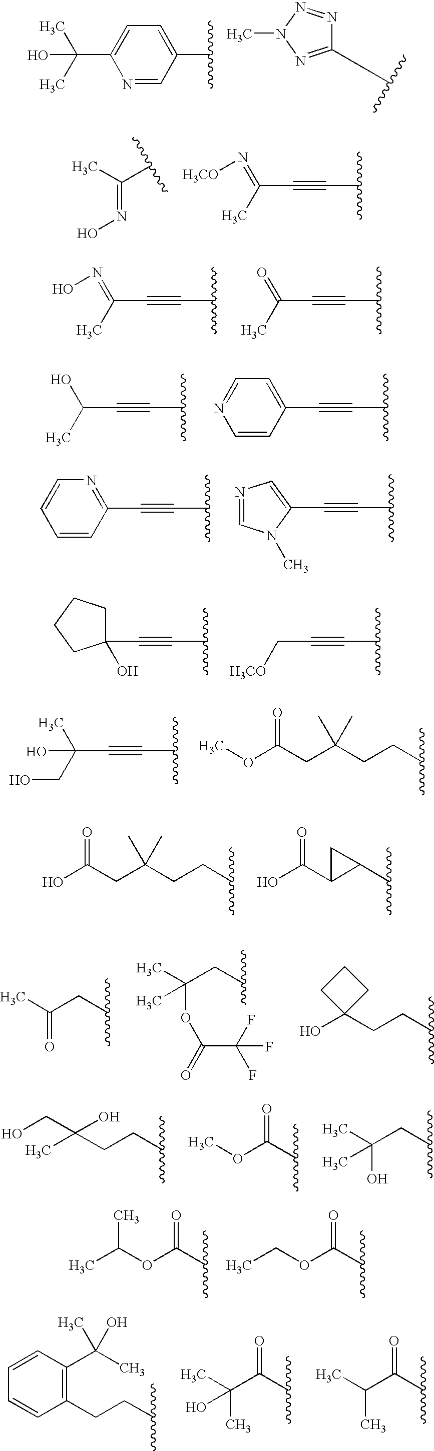 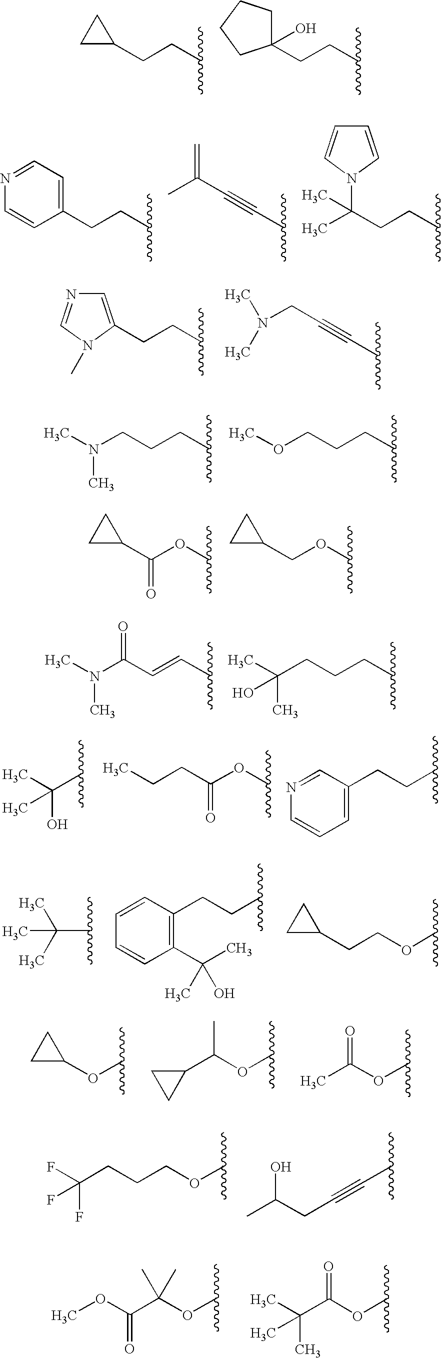
with the proviso that at least one of R³ or R⁶ is other than hydrogen.

26. The method according to claim 8 wherein the compound that inhibits microsomal prostaglandin E synthase-1 is represented by Formula B: 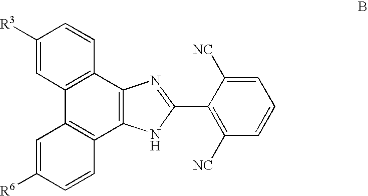
or a prodrug thereof, or a pharmaceutically acceptable salt of said compound or prodrug, wherein:
R³ is

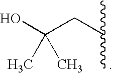

27. The method according to claim 26 wherein the compound that inhibits microsomal prostaglandin E synthase-1 is represented by Formula B wherein R⁶ is R¹²—O.

28. The method according to claim 27 wherein the compound that inhibits microsomal prostaglandin E synthase-1 is represented by Formula B wherein R¹² is selected from the group consisting of: (1) C₁₋₄alkyl and (2) C₃₋₆cycloalkyl-C₁₋₄alkyl-, wherein said C₁₋₄alkyl and C₃₋₆cycloalkyl may each be optionally substituted with 1 to 3 substituents independently selected from the group consisting of: OH, F, Cl, Br and I.

29. The method according to claim 26 wherein the compound that inhibits microsomal prostaglandin E synthase-1 is represented by Formula B wherein R⁶ is selected from F, Cl, Br and I.
